(19) United States
(12) Patent Application Publication
(10) Pub. No.: US 2003/0054370 A1
(43) Pub. Date: Mar. 20, 2003

(54) Systematic discovery of new genes and genes discovered thereby

(76) Inventors: Qiandong Zeng, Belmont, MA (US); Marco M. Kessler, Peabody, MA (US); Guillaume Cottarel, Arlington, MA (US)

Correspondence Address: BURNS DOANE SWECKER & MATHIS L L P, POST OFFICE BOX 1404, ALEXANDRIA, VA, 22313-1404, US

(21) Appl. No.: 10/083,357

(22) Filed: Feb. 27, 2002

Related U.S. Application Data
(60) Provisional application No. 60/271,406, filed on Feb. 27, 2001.
(60) Provisional application No. 60/333,726, filed on Nov. 29, 2001.

(51) Int. Cl. C12Q 1/68; C12Q 1/70; G06F 19/00

(52) U.S. Cl. 435/6; 435/5; 702/20

(57) ABSTRACT

The present invention is directed to a systematic in silico method to identify new coding sequences, including homologs of coding sequences, in S. cerevisiae and other organisms. The present invention is also directed to novel ORFs and the proteins encoded thereby identified using the in silico methods.

### CROSS-REFERENCE TO RELATED APPLICATIONS

This application claims priority under 35 U.S.C. § 119 to U.S. Provisional Application Nos. 60/271,406 entitled “Systematic Discovery of New Genes” filed Feb. 27, 2001 and 60/333,726 entitled “Systematic Discovery of New Genes and Genes Discovered Thereby” and filed on Nov. 29, 2001, the entire content of which are hereby incorporated by reference in their entirety.

### BACKGROUND OF THE INVENTION

The genomes of organisms are large stretches of DNA. In many organisms, the function of a great part of the genome is unknown since it does not contain encoded genes. Because of advances in computerization, genomic sequences are being deposited in public databases at a dramatic rate. However, this information will be of little value to biologists if the tools to manage and interpret the information are not available and are not reliable.

Today's scientists use advanced quantitative analysis and database comparisons to better manage the genetic information, and identify and define the relationship between sequences and the corresponding phenotypes. Increasingly, molecular genetics is shifting from the laboratory to the computer. However, the process of detecting genes in these sequences is still relatively slow.

One promising use of bioinformatics to increase the efficiency of research involves studying a genome to determine the sequence and relationship to other sequences and genes in the genome in other organisms. This information is of significant interest to pharmaceutical and biomedical research to, for example, assist in the evaluation of drug efficacy and resistance. Genetic databases for organisms such as Saccharomyces cerevisiae, Escherichia coli and Mycoplasma pneumoniae are publicly available, but the ability to manipulate this data is limited. To make the manipulation of genomic information easier, sophisticated databases and search programs have been developed.

Some well-known databases of genetic information include GenBank™, SwissProt and OMIM™ (Online Mendelian Inheritance in Man). GenBank™ is the National Institutes of Health (NIH) genetic sequence database, an annotated collection of all publicly available DNA sequences (Nucl. Acids Res. (2000) 28:15-8). There are approximately 10,336,000,000 bases in the 9,103,000 sequence records as of October 2000 (see www.ncbi.nlm.nih.gov/Genbank/). GenBank™ is part of the International Nueleotide Sequence Database Collaboration, which comprises the DNA DataBank of Japan (DDBJ), the European Molecular Biology Laboratory (EMBL), and GenBank™ at the NIH.

SwissProt is an annotated protein sequence database established in 1986 and maintained collaboratively by the Swiss Institute for Bioinformatics (SIB) and the European Bioinformatics Institute (EBI).

OMIM™ is a database catalog (www.ncbi.nlm.nih.gov/OMIM/) of human genes and genetic disorders authored and edited by scientists at The Johns Hopkins University. The database contains textual information and references, as well as links to MEDLINE and sequence records.

The Entrez retrieval system, run by the National Center for Biotechnology Information (NCBI) at the NIH, can search several linked databases at a time. Entrez can search biomedical literature databases, GenBank™, SwissProt and other protein databases, three-dimensional macromolecular structures and OMIM. Searches can produce results in the form of related sequences and structural neighbors.

A popular search program algorithm is BLAST (Basic Local Alignment Search Tool). BLAST is a set of similarity search programs designed to explore all of the available sequence databases regardless of whether the query is protein or DNA. The BLAST programs have been designed for speed, with a minimal sacrifice of sensitivity to distant sequence relationships. The scores assigned by a BLAST search have a well-defined statistical interpretation, making real matches easier to distinguish from random background hits. BLAST uses a heuristic algorithm which seeks local as opposed to global alignments and is therefore able to detect relationships among sequences which share only isolated regions of similarity (Altschul, S. F. et al. (1990) “Methods for assessing the statistical significance of molecular sequence features by using general scoring schemes,” Proc. Natl. Acad. Sci. USA, 87: 2264-2268).

Despite the strong computational biomolecular databases and search engines currently available, manual evaluation of the data produced is often required. Biological macromolecules exhibit many non-random features, most notably repetitive sequences and non-coding introns of genomic DNA. These typically require extensive evaluation of database matches that are found, which is a subjective, error-prone and tedious process. Present computational biology methods used to determine the number of coding sequences include promoter studies (Rainer, N. et al. (1999) Yeast 15:1775), codon usage (Staden, R. and McLachlan, A. D. (1982) Nucl. Acids Res. 10:141), or some combination of these methods. These procedures are based on current knowledge of gene function, and have a number of limitations.

In addition, there is evidence that the current computational methods for assessing coding potential often fail to identify open reading frames (ORFs) that are discovered through experimental and other non-computational methods. While sequence similarity search programs are a quick and versatile tool, frequently able to identify putative coding regions, the accuracy of the present methods is often compromised by factors such as differential and tissue-specific splicing, genes within genes (i.e., polycistronic coding domains) and the need for species specific parameters. From a statistical standpoint, the accuracy of known methods is extremely dependent on the choice of scoring system, statistical significance of alignments, sequence redundancy and the masking of confounding sequence regions.

For example, Serial Analysis of Gene Expression, or SAGE, is a technique designed to take advantage of high-throughput sequencing technology to obtain a profile of cellular gene expression. Essentially, the SAGE technique measures not the expression level of a gene, but quantifies a “tag”, which represents the transcription product of a gene. A SAGE tag is a nucleotide sequence of a defined length, directly 3′-adjacent to the 3′-most restriction site for a particular restriction enzyme. The data product of the SAGE technique is a list of tags, with their corresponding count values and thus is a digital representation of cellular gene expression. However, the SAGE method often sacrifices accuracy and fidelity in both the assignment of tags to genes as well as the ability to quantify a gene's expression level in order to increase throughput.

The need for an in silico (i.e., computational) method to identify new coding genes with the speed and versatility of the presently known methods, but with increased accuracy and lack of bias, is increasing exponentially in conjunction with the increasing accumulation of known sequences.

In addition to accurate methods, it is also important to have a model that lends itself well to research. In attempts to sequence and annotate the human genome, scientists have turned to the genomes of other organisms to use as models. One genome of one organism often used is that of the single-cell eukaryote, Saccharomyces cerevisiae (baker's yeast). Saccharomyces is amenable to genetic and biochemical manipulations, and many processes that occur in yeast also occur in larger eukaryotes, making yeast a model system for the study of eukaryotes, including humans. The yeast model system Saccharomyces cerevisiae was the very first eukaryotic genome to be completely sequenced (Goffeau, A. et al. (1996) Science 274:546) and is the subject of intensive research. The current consensus suggests the number of yeast genes, which are 100-amino acids or longer is in the range of 6000, (Goffeau (1996); Mewes, H. W. et al. (1997) Nature 387(6632 Suppl):7; and Winzeler, E. A. and Davis, R. W. (1997) Curr. Opin. Genet. Dev. 7:771, excluding a subset of small ORFs (Basrai, M. A. et al. (1999) Mol. Cell. Biol. 19:7041; and Velculescu, V. E. et al. (1997) Cell 88:243). Recent genetic studies designed to catalog all genome transcripts, using SAGE technology (Velculescu, V. E. et al. (1997)) and the analysis of a collection of transposon insertions (Ross-Macdonald, P. et al. (1999) Nature 402:413), have discovered new ORFs, which were not previously identified in silico. This pool of novel genes includes some putative proteins that are optimally shorter than 100 amino acids. However, determination of ORFs encoding polypeptides greater than 100 amino acids are also contemplated using the methods described herein.

### SUMMARY OF THE INVENTION

This invention relates to a systematic in silico method to identify new coding sequences, including homologs of coding sequences, in S. cerevisiae and other organisms. The method of the present invention compares ORFs of a first organism to a comprehensive database of sequences from related organisms to identify homologs. The results of this method using comprehensive database searches and experimental studies suggest that the number of coding genes in, for example, S. cerevisiae, is substantially higher than currently believed.

Another embodiment of the present invention comprises a method comprising the following steps:

(A) collecting genomic sequence of the first organism;

(B) identifying stop-to-stop ORFs of the first organism;

(C) translating the stop-to-stop ORFs into polypeptide sequences;

(D) comparing the polypeptide sequences of the first organism to amino acid translations of genomic libraries comprising genomes of other organisms; and

(E) identifying, based on sequence identity, ORFs of the first organism that are present in the other organisms, wherein the identified ORFs are coding ORFs. The ORFs are typically determined using the start codon AUG and stop codons UAA, UAG and UGA. However, the method also contemplates genome analysis with the less conventional start and stop codons discussed infra.

In one embodiment, the method comprises using BLAST with a p-value of less than 1. In another embodiment, FASTA is used, preferably with settings equivalent to those for BLAST with a p-value of less than 1.

In another embodiment, the invention comprises a method of identifying ORFs in a genome of a first organism comprising the steps of: (A) collecting genomic sequence of the first organism; (B) comparing the genomic sequence of the first organism to one or more other genomic libraries comprising genomes of other organisms containing ORFs; and (C) determining ORFs for the first organism based on the comparison. The ORFs or step B are ORFs that have been previously been described.

The nucleic acid and amino acid sequences of the organism being studied may have at least about 20%, more preferably 25%, and more preferably at least 30% sequence identity to known sequences.

The algorithm used would provide results equivalent to those obtained using BLAST wherein the p-value is less than 1.

The database may be a database of nucleotide sequences from a species related to the organism (e.g., S. cerevisiae and S. pombe) and a database of eukaryotic or prokaryotic nucleotide sequences. Specifically, the organism source of the eukaryotic nucleotide sequences may include, but is not limited to, primate, equine, bovine, caprine, ovine, porcine, feline, canine, lupine, camelid, cervidae, rodent, avian and ichthyes. The primate may be a human. Other organisms include vertebrates (e.g., mammals, birds, fish, and reptiles), invertebrates (e.g., worms), and plants.

In another embodiment, the organism can be a fungus of the phylum oomycota, chytridiomycota, zygomycota, ascomycota, basidiomycota or deuteromycota. Preferably, the fungus is yeast of the phylum ascomycota. More preferably, the yeast is the genus Saccharomyces or Schizosaccharomyces. Most preferably the yeast is the species S. cerevisiae or S. pombe.

The long genes are preferably about 100 or more amino acids in length. The smORFs preferably are less than about 100 amino acids, however, they can include polypeptides longer than 100 amino acids.

The smORFs isolated as described herein can be utilized in, for example, a microarray. For instance, a nucleic acid microarray is fabricated by high-speed robotics, generally on glass but sometimes on nylon or silicon substrates, for which probes with known identity are used to determine complementary binding. These arrays permit massive parallel gene expression and gene discovery studies. This technology allows researchers to monitor the whole genome on a single chip so that they have a better picture of the interactions among the thousands of genes simultaneously.

The present invention relates to smORF identified using the methods of the present invention, as well as a vector comprising the smORF and a cell comprising the vector. The cell preferably expresses the polypeptide encoded by the smORF. Further, the present invention relates to a nucleic acid that hybridizes to the sense or the antisense strand of the smORF, as well as an isolated polypeptide encoded by the smORF.

This invention also relates to 119 novel coding sequences (SEQ ID NOS: 1-119) from the S. cerevisiae genome discovered using the methods of the instant invention, or fragments thereof, and optionally, a sequence required for an amplification reaction. The fragment may be a primer. The invention further relates to an isolated polypeptide selected from the group consisting of SEQ ID NOS: 674-1346 and preferably SEQ ID NOS: 674-792, which appear to be expressed and in same instances, essential. The polypeptides should comprise at least 5 or 10 or more contiguous amino acid sequences of these sequences.

The present invention also relates to methods of modulating the genes and gene products identified using an in silico method described herein and identifying such modulating agents. Preferred modulating agents include antibiotics, antifungals and antisense agents. Modulating agents are generally a compound or compositions that modulates the biological activity of a gene, its transcript or the protein(s) encoded by that gene.

In another embodiment, the polypeptide or biologically active fragment thereof is in the form of a composition with a pharmaceutically acceptable carrier or excipient.

The present invention further relates to antibodies and immunologically active fragments thereof that recognize and bind to a smORF polypeptide or fragment thereof. These antibodies can be human antibodies, humanized or primatized® antibodies, monoclonal antibodies or bispecific antibodies. A further embodiment of the invention includes immunologically active fragments of the antibodies, such as Fab, Fab′, F(ab′)₂, Fv, scFv, and Fd.

### BRIEF DESCRIPTION OF THE DRAWINGS

FIG. 1 outlines the first steps of the strategy for new smORF identification using computational methods to identify new ORFs not identified by conventional methods.

FIGS. 2A-2E show the experimental validation of the S. cerevisiae smORFs. FIG. 2A shows the control experiments demonstrating that the RNA used for the RT-PCR experiment was not contaminated with genomic DNA. FIG. 2B shows the principle behind and the results of orientation-specific RT-PCR, thus demonstrating that the transcripts observed originate from the predicted DNA strand. FIGS. 2D and 2E show more examples of transcripts detected from the smORFs.

FIG. 3 shows three yeast smORFs, which have highly conserved homologs in other fungi and illustrates that two have highly conserved homologs in mammalian species. FIG. 3 shows the multiple sequence alignment of smORF8 (SEQ ID NO: 677) and its homologs, smORF139 (SEQ ID NO: 709) and its homologs, andsmORF570 (SEQ ID NO: 769) and its homologs. Abbreviations: Dm, Drosophila melanogaster; Hs, Homo sapiens; Ce, Caenorhabditis elegans; Sc, Saccharomyces cerevisiae; Ca, Candida albicans; Af, Aspergillus fumigatus; An, Aspergillus nidulans; Sp, Schizosaccharomyces pombe; Bt, Bos taurus; and Mm, Mus musculus. Residues that are identical or similar in all protein homologs are shaded in black and those identical or similar in two or more, but not all proteins in the alignment are shaded in gray. Homology shading was done with GeneDoc (Nicholas, K. B., et al. (1997), EMBnet News 4: 14).

FIG. 4 shows experimental evidence that smORF18 (SEQ ID NO: 4) codes for a polypeptide of the expected size. A triple HA-tag was fused to the C-terminal end of smORF18 using PCR, and the wild-type smORF18 gene was replaced by the tagged smORF18 gene by allele replacement into the chromosome. Soluble extracts were prepared and analyzed by Western blot analysis using monoclonal antibodies that recognize the HA epitope. Extracts from wild-type cells (lane 2) and extracts from two separate isolates carrying the HA-tagged smORF18 (lane 3 and 4).

FIG. 5. Human smORF18 homolog complementation of the temperature sensitive (ts) phenotype of the smorf18Δ strain. A yeast strain with a deleted smORF18 (smorfΔ) was transformed with plasmids carrying the wild-type yeast smORF18 (SEQ ID NO: 4), or the human smORF18 ORF under the control of the GAL1 promoter or empty vector. Transformants were then plated at 30° C. and 37° C.

FIG. 6. Diagram of smORF57 protein interaction map. The arrows indicate the orientation of each two-hybrid interaction.

### DETAILED DESCRIPTION OF THE INVENTION

I. Definitions

As used herein, the term “gene” refers to the fundamental physical and functional unit of heredity, which carries information from one generation to the next. A gene is a segment of DNA composed of a transcribed region and regulatory sequences that make possible transcription of the DNA.

As used herein, the term “organism” refers to eukaryotes and prokaryotes.

As used herein the term “known sequence” refers to a sequence (e.g., nucleic acid or amino acid) of any type publicly available and annotated.

As used herein, the term “long gene” refers to a gene that encodes a polypeptide of about 100 amino acids or more. Long genes can include genes encoding a polypeptide that is 100, 110, 120, 130, 140, 150, 175, 200, 300, 400, 500, 600, 750 and 1000 amino acids long or greater.

As used herein, the term “homolog” refers to a gene and protein coded thereby from one species with similarities to another gene and its encoded protein of the same species or among different species. These similarities can be based on structural (e.g., sequence similarity and/or three-dimensional commonality) and/or functional similarities (e.g., enzymatic and/or biochemical activity).

As used herein the term “ortholog” refers to a gene and protein encoded thereby from one species which corresponds to a gene and its associated protein in another species that is related via a common ancestral species (a homologous gene), but which has evolved to become different from the gene of the other species.

As used herein, the term “ORF” refers to an open reading frame, which corresponds to a nucleotide sequence that could potentially be translated into a polypeptide. For the purposes of this application, an ORF may be any part of a coding sequence, with or without stop codons. An ORF is usually not considered to be an equivalent to a gene locus until an mRNA transcript for a gene product is generated. The gene product can be detected and/or the ORF's protein product has been identified.

As used herein, the term “smORF” preferably refers to a small open reading frame that encodes a polypeptide of less than 100 amino acids. However, the methods of described herein can also be used to identify ORFs which encode polypeptides more than 100 amino acids long (e.g., 100, 125, 150, 200, 300, 400 500, etc. amino acids long). smORFs may encode a polypeptide of at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 and 100 amino acids. Preferably, smORFs encode polypeptides of 17 or 18 to 100 amino acids long. The nucleic acids encoding these polypeptides accordingly include nucleic acids that are 15 to 300 nucleotides in length or any number of nucleotides between that range. The nucleic acid can be any that encodes the identified smORF protein, including synthetic nucleic acids and the wild-type nucleic acid. Preferred nucleic acids will have at least 8 contiguous nucleotides. However, other nucleic acids may have from 8 to 300 or more contiguous nucleotides, or any number lying within that range (e.g., 25, 75, and the like).

As used herein, “annotation” refers to the description of the properties of a given sequence or gene, such as the protein encoded by the gene, function of the protein, its domain structure, post-translational modifications, variants, etc.

As used herein, the term “in silico” refers to a computational method of analyzing nucleic acid and/or amino acid sequences.

As used herein, the term “sequence identity” refers to the relatedness of two genetic sequences, as represented by the percentage of the amino acids and/or nucleotides they share.

As used herein, the term “sequence homology” defines regions of DNA sequence, which are the same at different locations of the genome, or between different DNA molecules such as between the genome and a plasmid or DNA fragment.

As used herein, the term “microarray” (also referred to as “biochip” and “DNA chip”) refers to a microarray comprising nucleic acids. A microarray is fabricated by high-speed robotics, generally on glass but sometimes on nylon or silicon substrates, for which probes with known identity are used to determine complementary binding, thus allowing parallel gene expression and gene discovery studies. This technology allows researchers to monitor the whole genome on a single chip so that they have a better picture of the interactions among the thousands of genes simultaneously.

As used herein, the term “fragment thereof” refers to an incomplete and/or spliced section of the smORFs of the present invention. By “biologically active” is meant that portion of the smORF that retains biological activity. For example, for a nucleic acid, it might be the activity of binding to a cognate strand. With reference to a polypeptide, by biologically active is meant that portion which is, for example immunogenic or has an antigenic epitope, or that has enzymatic activity.

As used herein, the term “false positives” refers to a test result, which erroneously assigns the test subject to a specific group, due to insufficiently exact methods of testing.

As used herein, the term “false negatives” refers to a test result, which excludes the test subject from a specific group, due to insufficiently exact methods of testing.

As used herein, the term “hits” refers to when a database/computer reviews the information cache stored therein and finds data meeting the chosen parameters; the result is called a “hit.”

As used herein, the term “ESTs” (“expressed sequence tags”) refers to a short strand of DNA, which is part of a cDNA. Because an EST is usually unique to a particular cDNA, and because cDNAs correspond to a particular gene in the genome, ESTs can be used to help identify unknown genes and to map their position in the genome.

As used herein, the term “RT-PCR” refers to reverse transcriptase-polymerase chain reaction. In this process, mRNA is subjected to reverse transcriptase, resulting in the production of cDNA complementary to the mRNA. Large amounts of selected cDNA can then be produced by means of the polymerase chain reaction.

As used herein, the term “database” refers to a large collection of genetic data organized especially for rapid search and retrieval by computer.

As used herein, the term “algorithm” refers to a step-by-step procedure for solving a problem or accomplishing some end, especially by a computer. Specifically, the term “algorithm” refers to a search algorithm used to locate specific data from a genetic database.

As used herein, the term “amplification reaction” refers to a reaction causing an increase in the number of copies of a specific DNA fragment, such as the polymerase chain reaction (PCR).

The polypeptide of the present invention is preferably in an isolated form. As used herein, the term “isolated polypeptide” refers to a polypeptide removed from its native environment. Thus, a polypeptide produced and contained within a recombinant host cell would be considered “isolated” for the purposes of the present invention. Also intended as an “isolated polypeptide” are polypeptides that have been purified, partially or substantially, from a recombinant host. Similarly, by “isolated nucleic acid” or “isolated polynucleotide” is meant a nucleic acid sequence, which is purified from other nucleic acid and protein contaminants.

As used herein, the term “NrProtein database” refers to the non-redundant protein database, one of the databases available for searching using the BLAST algorithm.

The present invention is directed to methods of identifying new genes in the genome of an organism. The method comprises the steps of removing all annotated ORFs and long genes from the organism's genome and then isolating small ORFs (smORFs) of preferably less than 100 amino acids. These smORFs have at least a 20% sequence identity to all known sequences from related organisms, determined by searching a database using a search algorithm. The methods may further comprise the steps of identifying the smORFs that are coding ORFs and verifying that the smORFs can transcribe RNA using molecular genetics tools.

The present invention is also directed to 119 novel ORFs (SEQ ID NOS: 1-119) and their corresponding proteins (SEQ ID NOS: 674-792) from the S. cerevisiae genome, which were identified through the methods of the present invention as set froth in Table 2. The present invention is also directed to 554 other ORF sequences (SEQ ID NO: 120-673) and their corresponding proteins (SEQ ID NOS: 793-1346) identified in S. cereviseae using the disclosed in silico method (see Table 2).

II. Identification of Novel Coding Sequences

This invention relates to methods of identifying novel coding sequences in an organism, for example, S. cerevisiae, as well as in other prokaryotic and eukaryotic organisms. The methods of the present invention would be appropriate for use on the genome of any organism, including, but not limited to, plants (e.g., rice, maize, Aribidopsis), the plant pathogen Phytophthora, invertebrates (e.g., nematodes, higher worms, fruit flies, etc.), fish (e.g., zebrafish) mammals (e.g., mice, humans, etc.) and any of the other organisms discussed herein.

One method of identifying new genes in the genome of an organism comprises the steps of removing annotated ORFs and long genes, preferably all known sequences, from the organism's genome, and then isolating small ORFs (smORFs) comprising nucleic acid and amino acid sequences, preferably predicted amino acid sequences having at least a 20% sequence identity to all known sequences, more preferably amino acid sequences from related organisms, wherein percent identity is determined using an algorithm with parameter settings consisting essentially of or equivalent to a p-value of less than 1 used in conjunction with a BLAST algorithm to search a database of genetic information.

Preferably, the methods of the present invention are especially adaptable for whole fungal genomes. More preferably, the fungus is yeast. Most preferably, the yeast is S. cerevisiae or C. albicans. Accordingly, one embodiment of the present invention is a method of identifying new genes in the genome of S. cerevisiae comprising the steps of removing all annotated ORFs and long genes from the S. cerevisiae genome, and then isolating small ORFs (smORFs) comprising predicted amino acid sequences having at least a 20% sequence identity to all known fungal amino acid sequences, wherein percent identity is determined using an algorithm. For example, if the algorithm is BLAST the parameters comprise a p-value of less than 1. Other algorithms contemplated would use parameters producing similar results as would be known to the artisan of ordinary skill.

A comparison of the yeast S. cerevisiae ORFs with a comprehensive fungal database (excluding S. cerevisiae) suggest that most budding yeast ORFs have homologs in other fungi. This led to the conceptualization and validation of a new process for identifying novel coding sequences. For example, this would include the following steps:

1. Take one nucleic acid genome of an organism to probe (e.g., S. cerevisiae).

2. Collect known nucleic acid sequences (e.g., genes) of the genome from step 1.

3. Optionally remove known genes.

4. Optionally take the portions of genome remaining after the above steps (known or otherwise, but not known to contain genes, e.g., intergenic regions).

5. Take either intergenic region or whole genome.

6. Identify all open reading frames (ORFs) of preferably about 17 amino acids or longer stop-to-stop.

7. Perform a six-frame translation (three frames forward, and three frames backward to correspond to the complementary strand).

8. Look for stop codons (*). Start counting residues right after the stop codon to the next stop codon. Take all the sequences that are preferably 17 amino acids or longer and call it an ORF (stop-to-stop). Typically, most programs identify sequences of at least 50 to 60 amino acids or longer.

9. The novel step is then to construct a comprehensive database containing genomic DNA and cDNA sequences from as many organisms related to the subject as possible. For example, if the subject organism is S. cerevisiae, the database would include genomic and EST sequences from as many fungal species (excluding S. cerevisiae) as available in the public and/or private databases, including C. albicans, Aspergillus nidulans, A. fumigatus, Schizosaccharomyces pombe, Neurospora crassa, Cryptococcus neoformans, Fusarium sporotrichioides, etc.

10. The ORFs identified in steps 7 and 8 are then compared against a six-frame translation of the nucleotide sequences contained in the database described in step 9. For example, if the organism being studied is S. cerevisiae, then the ORFs identified in step 6 are compared against the nucleotide sequences in the fungal database. Preferably, a comparison algorithm, such as TBLASTX is used. In the instance of TBLASTX, the parameters preferably include a p-value of less than 1. Comparable algorithms with comparable parameters can also be utilized.

11. Compare the amino acid sequences using sequence identity parameters.

12. Collect all the hits against entries in the database (e.g., fungi).

13. A hit determines whether the ORF being studied from the first organism (e.g., S. cerevisiae) is likely to be a coding ORF (i.e., smORF), because it has predicted homologs in the organisms contained in the database (e.g., fungal database).

A. Compilation of Organism Genome and Removal of Annotated ORFs

For an ORF to be considered to be a good candidate for coding a cellular protein, a minimum size requirement is often set. This is not the case here. One novel characteristic of the present invention is that the small ORFs, which are often discounted in genome analysis, are considered here.

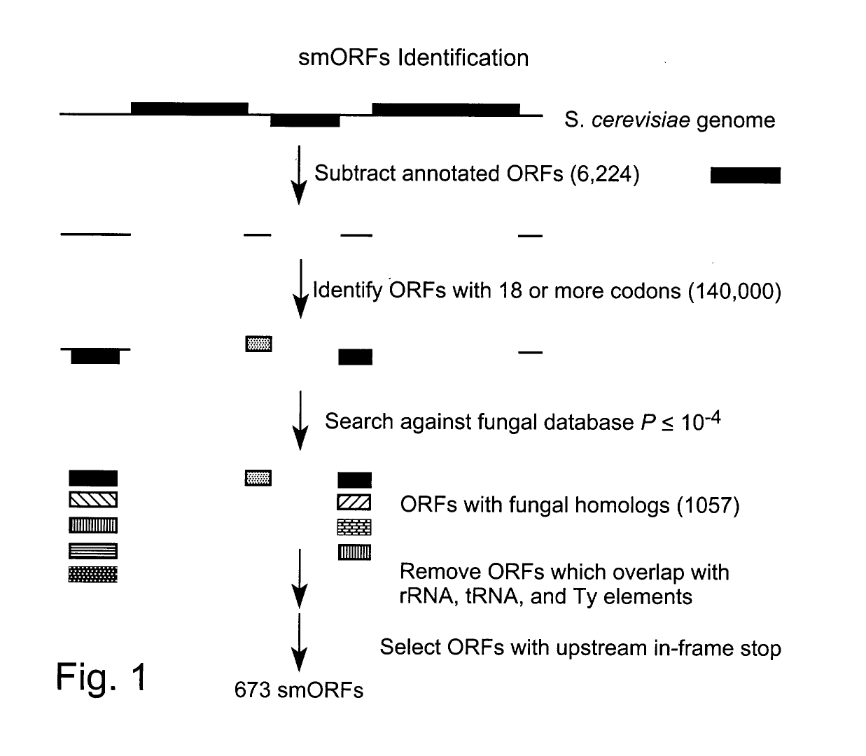

The first step in the methods of the present invention is an examination of the entire genome of the organism of choice, as outlined in FIG. 1. The sequences of the genome of choice may be found anywhere, including, but not limited to, GenBank™, EST sequence databases, Celera's recent human genome database (Venter et al., “The Sequence of the Human Genome,” Science 291: 1304-51 (2001)), and other organism genome databases as they are elucidated. For example, the entire S. cerevisiae genomic sequence (12.07 mb total) was examined, and obtained from the Saccharomyces Genome Database as of Dec. 5, 1997. (See http://genome-www.stanford.edu/Saccharomyces/).

B. The Isolation of smORFs Using Bioinformatics

The next step in the method of the claimed invention is the isolation of smORFs, by running the remaining ORFs obtained in the above steps against a database of known genes to identify any potential homologs. The database can be any searchable database, which can identify homologous sequences. Preferably the databases are compared using algorithms such as BLAST or FASTA or equivalent algorithms.

Specifically, a method of identifying new genes in the genome of an organism comprises the steps of removing all annotated ORFs and long genes from the organism's genome. Alternatively, the removal of sequences does not need to occur. This is followed by isolating small ORFs (smORFs) comprising nucleic acid and amino acids sequences having at least a 20% sequence identity to all known sequences from related organisms. Preferably, the comparison is of amino acid sequences.

The smORFs may have a sequence identity to all known sequences from related organisms of about 20% or more. Preferably, the sequence identity is at least about 25% sequence identity and more preferably at least about 30% sequence identity.

The first organism database searched and compared to another organism may comprise a plurality of known genomic nucleotide sequences and expressed sequence tags (ESTs). For example, the nucleic acid encoding the polypeptide sequences of the present invention are analyzed using BLAST, against any type of sequence from similar organism, including, but not limited to, nucleotide sequences, protein sequences, peptide sequences and ESTs.

In this step, the database should be a database of nucleotide sequences from a species related to the organism of choice. For example, the genome of the yeast S. cerevisiae was searched against a database of all known fungal sequences. Alternatively, the database may be a database of all eukaryotic nucleotide sequences. Specifically, the organism source of the eukaryotic nucleotide sequences may include, but is not limited to, primate, equine, bovine, caprine, ovine, porcine, feline, canine, lupine, camelid, cervidae, rodent, avian and ichthyes. If a primate database is searched, the primate is preferably human.

The long genes removed from the genome are all genes of about 100 or more amino acids. The small ORFs (smORFs), the preferred sequences of interest in the present invention, are sequences of typically less than 100 amino acids. However, the methods of the invention can be used to identify ORFs, which encode polypeptides greater than 100 amino acids. One of the novel features of the instant invention is the focus on ORFs, which are small and therefore previously excluded or not rigorously studied by researchers.

For example, in the present invention, the S. cerevisiae genome was analyzed and the nucleotide sequences of the previously identified 6,224 coding ORFs were removed. Next, the remaining sequences (3.45 mb) were analyzed to identify all stop-to-stop ORFs using a size of preferably about 17 or 18 residues or longer based on the fact that in E. coli, the overwhelming majority of genes code for proteins of preferably about 17 or 18 amino acids or longer (E. coli Genome Center, Oct. 13, 1998, revision date, University of Wisconsin, Madison). http://www.genetics.wisc.edu/). This analysis produced approximately 140,000 ORFs, most of them shorter than 100 residues.

In isolating smORFs of an organism's genome, a microarray may be used.

In one embodiment of the present invention, the ORFs thus identified were searched against a comprehensive fungal sequence database to identify any ORFs with potential homologs. This fungal database consisted of all NCBI entries listed under “fungi” (Aug. 20, 2000, excluding any S. cerevisiae sequences), plus the genomic sequences from Candida albicans (Stanford University) and Aspergillus fumigatus (PathoGenome™ database) (A. fumigatus genomic sequences are available at http://www.LabOnWeb.com), EST sequences from Aspergillus nidulans, Cryptococcus neoformans, Fusarium sporotrichioides, and Neurospora crassa (University of Oklahoma Health Sciences Center), and Pneumocystis carinii EST sequences (University of Georgia). Using a cutoff score of p→10⁻⁴ (a score of p→10⁴ was chosen, since it is reasonably stringent for small ORFs), 1057 S. cerevisiae ORFs were identified with potential homologs in the fungal database. Preferably the p value when using BLAST is a value less than 1. After removing smORFs overlapping with rRNA, tRNA and retrotransposon elements (i.e., TY elements), 673 smORFs were obtained (SEQ ID NOS: 1-673). Since homologs of these budding yeast ORFs were found in at least one other fungal species, it seems reasonable to predict that most of these 673 ORFs (SEQ ID NOS: 1-673) are likely to be coding ORFs (FIG. 1) as further described in Table 2.

Table 2 describes the function of the genes and proteins of the present invention. The first column contains the smORF designation number. The nucleotide and amino acid sequences designated by their SEQ ID NOS are contained in the second and third columns. The corresponding length of the nucleotide and amino acid sequences are listed in the fourth and fifth columns, respectively. BLAST scores and probabilities from the described analysis herein are provided in the sixth and seventh columns, respectively. The description of the gene and protein is contained in the eighth column. The description field provides, where available, the accession number (AC) or SwissProt accession number (SP), the locus name (LN), Superfamily classification (CL), the organism (OR), the source of variant (SR), the E.C. number (EC), the gene name (GN), the product name (PN), the function description (FN), the map position (MP), left end (LE), right end (RE), coding direction (DI), the database from which the sequence originates (DB), and the description (DE) or notes (NT) for each ORF.

C. Validation of the Novel Coding Sequences

Finally, the smORFs identified using the methods of the present invention may be validated as coding sequences able to transcribe RNA by the use of known experimental techniques such as reverse transcriptase-polymerase polymerase chain reaction (RT-PCR). A subset (i.e., 154) of the 673 smORFs (SEQ ID NOS: 1-673) were chosen for analysis by RT-PCR. RT-PCR analysis showed that a transcript could be demonstrated with 119 smORFs (SEQ ID NOS: 1-119). With regard to any smORFs identified and validated through the methods described above, the present invention further relates to a vector comprising such a smORF, a cell comprising the vector, a polypeptide encoded by the smORF and a nucleic acid which hybridizes to the sense or antisense strand of a smORF identified using the methods of the present invention, preferably under stringent conditions.

Stringency is a term used in hybridization experiments to denote the degree of homology between the probe and the filter bound nucleic acid; the higher the stringency, the higher percent homology between the probe and filter bound nucleic acid. If the stringency is too low, unspecific hybridization may occur. If the stringency is too high, only a weak or no signal may be observed. For any hybridization, stringency can be varied by manipulation of three factors: temperature, salt concentration, and formamide concentration; however, stringent conditions are sequence-dependent and will differ depending on the circumstances. For example, longer sequences hybridize specifically at higher temperatures. Generally, highly stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature at which 50% of the probes complementary to the target hybridize to the target sequence at equilibrium. Stringent conditions will be those in which the salt concentration is less than about 1.0 M sodium ion, typically about 0.01 to 1.0 M sodium ion concentration (or other salts) at pH 7.0 to 8.3, and the temperature is at least about 30° C. for short probes (e.g., about 10 to about 50 nucleotides) and at least about 60° C. for long probes (e.g., greater than about 50 nucleotides). Stringent conditions may also be achieved with the addition of destabilizing agents such as formamide.

The degree of hybridization may also depend the amount of identity between the sequences. Preferably the region of identity is greater than about 5 bp, more preferably the region of identity is greater than 10 bp.

Stringent hybridization conditions are known in the art and include, but are not limited to: (a) washing with 0.1× SSPE (0.62 M NaCl, 0.06 M NaH₂PO₄.H₂O, 0.075 M EDTA, pH 7.4) and 0.1% sodium dodecyl sulfate (SDS) at 50° C.; (b) washing with 50% formamide, 5× SSC (0.75 M NaCl, 0.075 M sodium citrate), 50 mM sodium phosphate (pH 6-8), 0.1% sodium pyrophosphate, 5× Denhardt's solution, sonicated salmon sperm DNA (50 μ/ml), 0.1% SDS and 10% dextran sulfate at 42° C., followed by washing at 42° C. in 0.2× SSC and 0.1% SDS; and (c) washing with 0.5 M NaPO₄, 7% SDS at 65° C. followed by washing at 60° C. in 0.5× SSC and 0.1% SDS. High stringency hybridization conditions are those performed at about 20° C. below the melting temperature (Tₘ). Preferred stringency is performed at about 5-10° C. below the melting temperature (Tₘ). Additional hybridization conditions can be prepared as found in chapter 11 of Sambrook et al., (1989) Molecular Cloning: A Laboratory Manual, 2d Ed. Cold Spring Harbor Laboratory Press, or as would be known to the artisan of ordinary skill.

Extensive guides to the hybridization of nucleic acids and sequence identity can be found in Sambrook et al., (1992) Molecular Cloning: A Laboratory Manual, 2d Ed. Cold Spring Harbor Laboratory Press and Ausubel et al., (1995) Current Protocols in Molecular Biology, Greene Publishing Co., NY.

We have developed and validated a novel method for gene identification in sequenced genomes and used it to identify new genes in S. cerevisiae. With this method, one should be able to find new coding ORFs in S. cerevisiae or other yeasts by simply searching potential budding yeast ORFs against other fungal species. Even though our experimental design was purposely non-exhaustive to demonstrate the proof of principle and the validity of this gene discovery process, we found strong evidence for several hundred new genes in the S. cerevisiae genome. For the three new genes selected for detailed analysis and experimental studies, we identified orthologs in other fungal species, as well as in other eukaryotes (e.g., mammals). This example can be expanded to include smORFs that partially overlap with annotated ORFs and smORFs that are completely located within previously annotated ORFs. The identification of conserved genes across a wide range of species provides the opportunity to use S. cerevisiae and/or other fungi to study the function of their counterparts in humans. In addition, the disclosed methods can be applied to other sequenced genomes, including humans, in order to identify coding ORFs not previously detected using conventional methods. This novel genome comparison approach to identify new ORFs will accelerate genome annotation and gene identification.

III. Novel smORF Sequences Identified

To establish a proof of principle and verify this new method, a case study was done using the budding yeast genome, because it is one of the most exhaustively studied biological systems. Consequently, analysis of this genome to identify new genes not previously described is a rigorous test of the system, challenging the present methods used to identify new genes.

The new smORFs identified using the methods described herein were then subjected to a validation step. A comprehensive analysis of the three smORFs was performed as a means of verifying their ability to encode a polypeptide. Most of the analysis was done with the Compas™ package (Genome Therapeutics Corporation), which performs a database search, as well as identification of such structural elements as motif, protein family (pfam), helix-turn-helix, coiled-coil and signal peptide to name a few; Compas™ also identifies protein secondary structure and predicts cellular location. We identified a wide range of homologs in other species for all three smORFs. SmORF18 and smORF570 have homologs in fungi and mammals (FIG. 3). SmORF18 also has plant homologs. Homologs of smORF139 were found only in fungi so far (FIG. 3). SmORF18 seems to be part of a larger protein in Arabidopsis thaliana, Sorghum bicolor, Oryza sativa, Glycine max and other plants, but the orthologs in human, Caenorhabditis elegans, Drosophila melanogaster, and Schizosaccharomyces pombe are about the same length as the S. cerevisiae smORF.

While the patches of highly conserved residues in the homologs for the three smORFs strongly suggest that these ORFs encode proteins, the definitive proof came from experimental work, wherein molecular genetics tools were used to confirm that these smORFs transcribe RNA. Primers were designed to amplify the three smORFs as well as the ACT1 gene (actin) control. The primers were chosen to give a PCR amplification product of 250 to 300 base pairs that lies inside the ORFs. Examples of primers for the ACT1 gene and three smORFs are shown in Table 1. These primers were used for PCR amplification of S. cerevisiae Genomic DNA (template) to test the PCR amplification conditions (Yeast genomic DNA was prepared from strain W303 using the Yeastar Genomic DNA kit (Zymo Research) as suggested by the manufacturer.

TABLE 1

| | | SEQ |
|---|---|---|
| | | ID |
| smORF | Primer Sequence | NO |
| smORF18 | 5′-TGACGAAATCGAAATCGAAG-3′ | |
| | 5′-GATGCCTGCCTCTTCGTAGT-3′ | |
| smORF139 | 5′-TGCCTAAGAGATTAAGTGGGTT-3′ | |
| | 5′-CGTCAGTTCAGGGTGTGAAA-3′ | |
| smORF570 | 5′-TGTCTGCATTATTTAATTTTCGTTC-3′ | |
| | 5′-AGCTGTTAAATTGACTGATGGC-3′ | |
| yeast ACT1 gene | 5′-TGTCACCAACTGGGACGATA-3′ | |
| | 5′-AACCAGCGTAAATTGGAACG-3′ | |

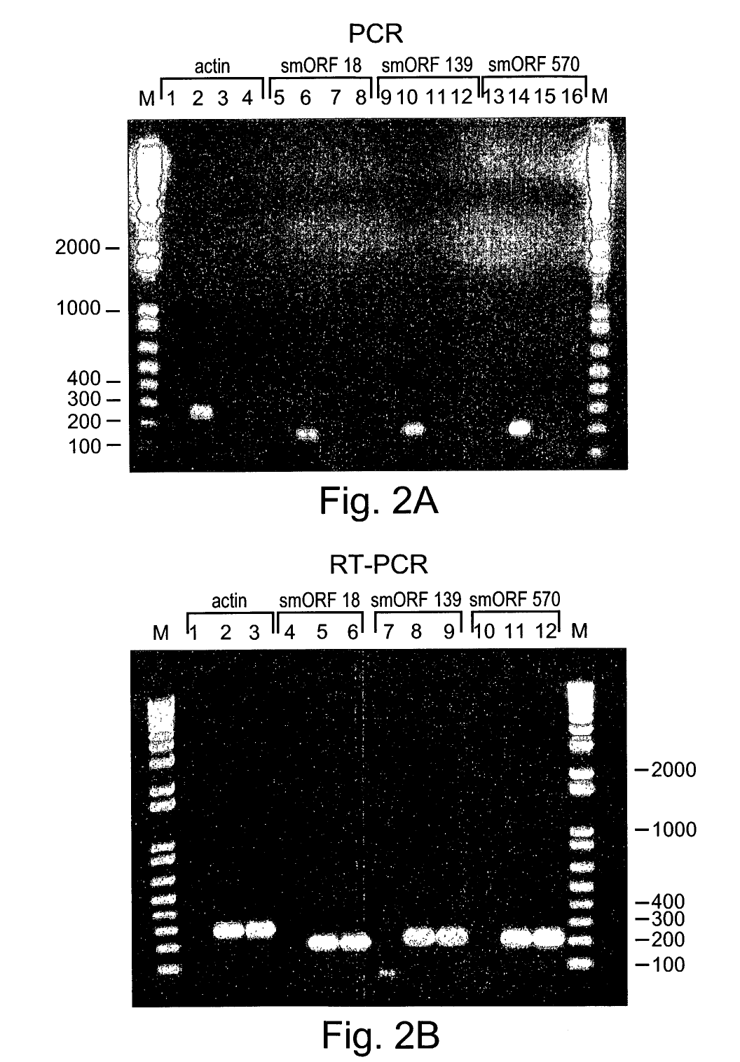

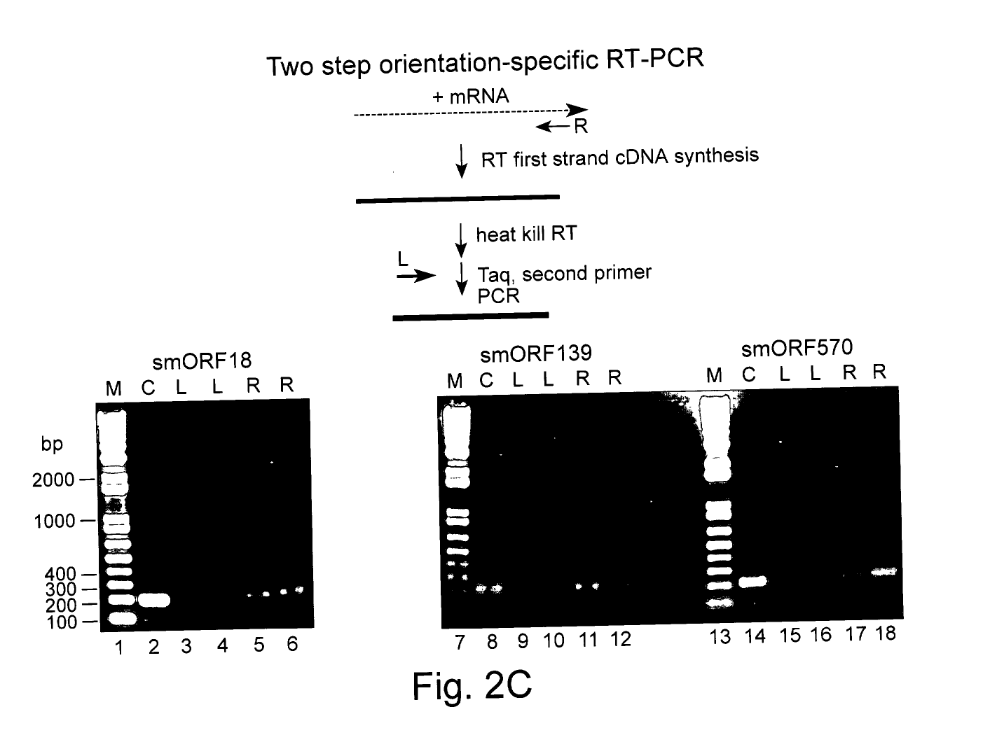

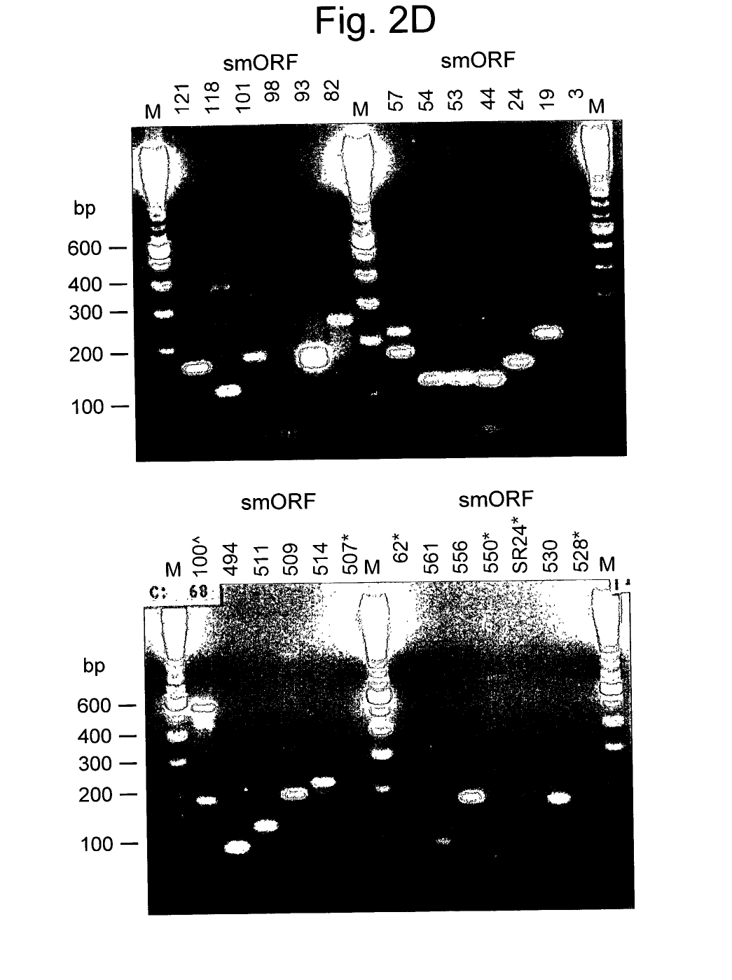

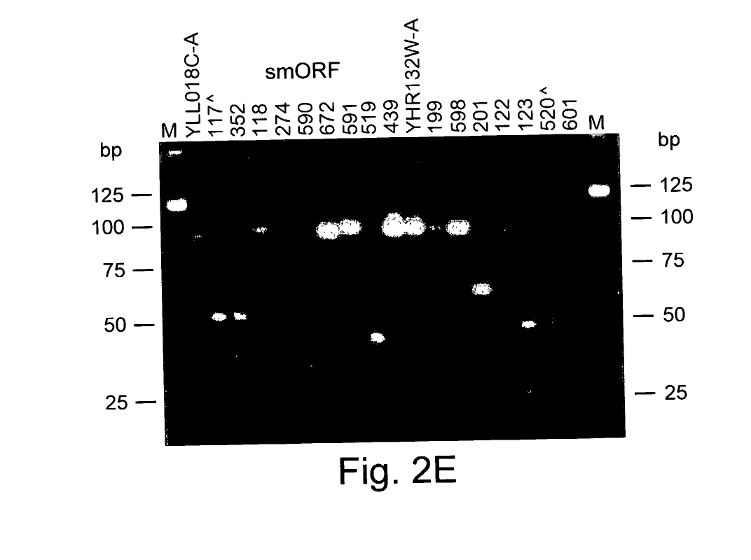

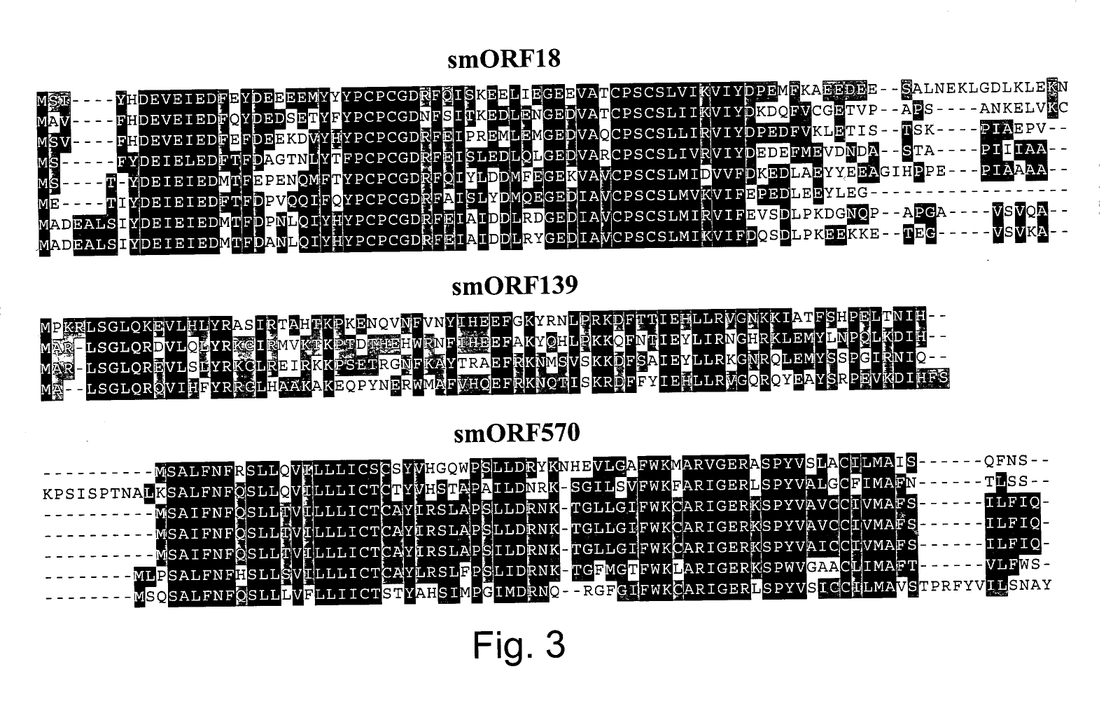

Products of the predicted size were obtained for all three smORFs, as well as the actin control (FIG. 2A, lanes 2, 6, 10, and 14). No PCR products were obtained in reactions without template (FIG. 2A, lanes 1, 5, 9, and 13), or using RNA isolated from S. cerevisiae grown on rich media (YEPD) or complete synthetic minimal (CSM) media (FIG. 2A, lanes 3, 4, 7, 8, 11, 12, 15, and 16). This indicates that these RNA samples were not contaminated with genomic DNA (RNA was isolated from 5×10⁷ yeast (strain W303) cells growing exponentially in YEPD or synthetic complete minimal media using the RNeasy™ Mini kit from Qiagen including a DNase (Roche) digestion step.) We then tested for the presence of RNA transcripts originated from these smORFs, as well as from the actin control using RT-PCR (RT-PCR reactions were done with the OneStep RT-PCR Kit from Qiagen as recommended by the manufacturer). Products of the expected sizes were obtained for actin, as well as all three smORFs (FIG. 2B, lanes 2, 3, 5, 6, 8, 9, 11, and 12). This indicates that actin and the three smORFs are indeed expressed in yeast cells grown in both rich and in minimal media. No RT-PCR product was obtained in reactions without template (negative control) (FIG. 2B, lanes 1, 4, 7, and 10). The identity of the RT-PCR products was confirmed by cloning. The RT-PCR products were isolated from an agarose gel and then cloned into pCR21-TOPO (Invitrogen), as recommended by the manufacturer. The sequences were then restriction mapped and dideoxy sequenced.

To determine whether the identified smORFs were indeed transcribed from the predicted DNA strands, a modified RT-PCR experiment was performed. First, primer complementary to the predicted mRNA and the reverse transcriptase were added. After first strand cDNA synthesis, the reverse transcriptase was inactivated with heat. Taq polymerase and both smORF-specific primers were then added (FIG. 2C). Under these conditions, PCR products were observed only when first strand synthesis was conducted with primers complementary to the predicted mRNA (lanes 5, 6, 11, 12, 17 and 18). No PCR product was observed if first strand synthesis was done with primers that have the same sequence as the mRNA (lanes 3, 4, 9, 10, 15 and 16). These results indicate that the transcripts observed for smORFs 18, 139 and 570 (SEQ ID NOS: 4, 36 and 96) are made from the predicted strand. This same study was extended to 151 additional smORFs, most of which have a potential homolog in the genome of C. albicans. The results show that a RT-PCR product of the expected size was obtained for 116 of these smORFs (FIGS. 2D and 2E). Therefore, 119 of the 154 smORFs are transcribed from the predicted DNA strand (Table 2). See SEQ ID NOS: 1-119.

To address the possibility that the observed smORF transcripts were products of read-through transcription from genes located upstream from the smORFs, the RT-PCR experiment was conducted using a primer complementary to the mRNA for first strand synthesis (FIG. 2C) and with a second primer located 400 base pairs upstream of the smORF. Under these conditions, no RT-PCR product was observed demonstrating that the smORF transcripts were not the result of read-through transcription from upstream genes.

Functional analysis can then be performed. For example, site-directed mutagenesis can be performed to disrupt the function of each gene and examine the resulting phenotypic changes, as would be known to the artisan of ordinary skill. The three smORFs described here do not overlap with previously annotated ORFs and a clear start-to-stop ORF can clearly be defined. These three ORFs are not duplicated on the budding yeast genome, as only one copy of each ORF was identified in the genome. Additionally, these S. cerevisiae smORFs have highly conserved homologs in other fungal species (50 to 60% amino acid identity and 70 to 80% similarity). In the case of smORFs 18 and 570 (SEQ ID NOS: 677 and 769, respectively) highly conserved homologs could also be found in mammalian genes.

The yeast smORFs identified using the methods described herein are described more fully below.

(i) Yeast smORF570. Comprehensive bioinformatics analysis of the yeast smORF570 protein sequence (SEQ ID NO: 769) suggests that this protein functions as a secreted protein. Using SigCleave (eGCG version 8), we have identified three overlapping signals with scores of 11.6, 6.4 and 5.1, in a region that extend from amino acid 9 through amino acid 29, with a predicted cleavage site in the region of amino acids 22-27. Although TopPredII suggests the presence of two transmembrane domains with moderate certainty, the initial domain identified overlaps the SignalPeptide prediction noted earlier and likely represents the hydrophobicity associated with the SignalPeptide region. Given the presence of three conserved cysteine residues within the protein, which are likely to represent sites of inter- or intra-protein cross-linking, the second site identified by TopPredII is sub threshold (below a certainty cut-off of 1.5) and is more consistent with hydrophobicity that drives protein folding rather than a membrane spanning region. Taking these data together, our analysis would support the function of smORF570 as a secreted protein that could act as either a ligand, a soluble receptor or a binding protein. Based on this information, smORF570 would also be a target for antifungal agents and other therapeutics described herein.

The human homolog of smORF570 maps to Chromosome 19 (19q13.1), in a region with multiple olfactory receptors (AC005255, between OLFR and MEL), though the gene itself was not identified. The human smORF570 protein is 74% identical to its D. melanogaster homolog (AE003512), 39% identical to its C. elegans counterpart, and 40% identical to a novel gene expressed in human adrenal gland (AF164793). EST hits for the human smORF570 homolog were found with bovine placenta, pig spleen lambda, mouse irradiated colon, and embryonal carcinoma cell line F9. Based of this information, the human homolog is most likely involved in cancer and could act as a target as a therapeutic target.

(ii) Yeast smORF18. Of particular note is the sequence conservation (31%) share in common with the N-terminus of a chicken fas ligand receptor-soluble form (AF296875, 285 amino acids, p=0.84). The number and spacing of Cys residues are also similar in the aligned portion of the two proteins. EST hits were found in mouse placenta, Beddington mouse dissected endoderm, rat kidney, rat embryo, and human placenta.

The conservation of residues across fungi suggests that smORF18 could be used as an antifungal target using the methods described herein. The identity between human smORF18 homolog and its counterparts in D. melanogaster, C. elegans, A. thaliana are 70%, 69% and 60%, respectively, at amino acid residue level. SmORF18 protein is also 31% identical to Schizosaccharomyces pombe dnaj heat-shock protein (316 amino acids).

To further demonstrate the validity of the method, a comprehensive analysis of smORF18 was conducted. A wide range of homologs was identified in other species (FIG. 3). SmORF18 seems to be part of a larger protein in Arabidopsis thaliana, Sorghum bicolor, Oryza sativa, Glycine max and other plants. The human, Caenorhabditis elegans, Drosophila melanogaster and Schizosaccharomyces pombe smORF18 homologs are about the same size as the S. cerevisiae smORF18 (SEQ ID NO: 677). SmORF18 (SEQ ID NO: 4) was recently annotated by Blandin et al., (FEBS Lett. 487: 31, 2000) and assigned the systematic name YBL071W-A.

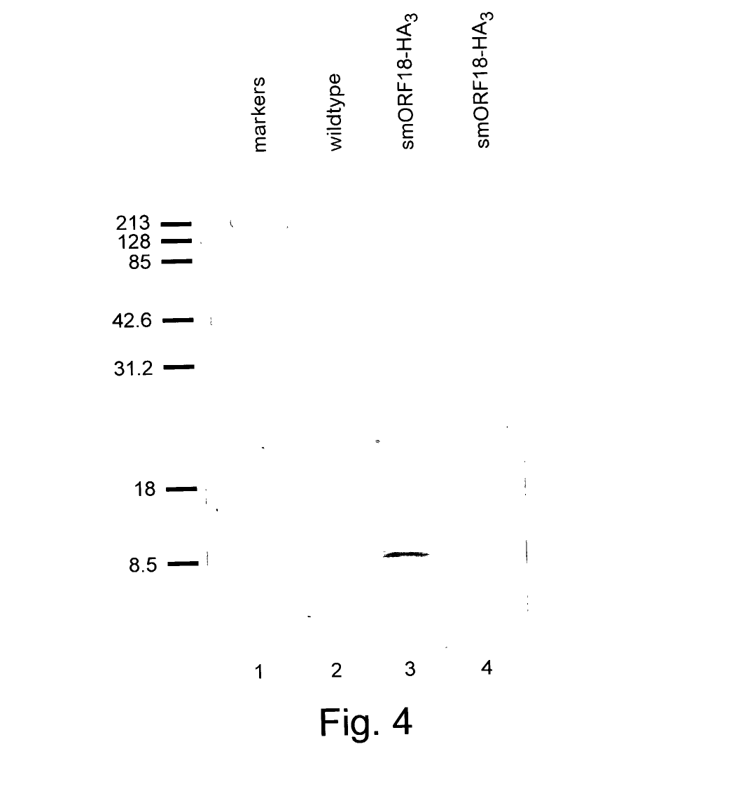

Study of smORF18 (SEQ ID NO: 4) was extended to determine whether a protein product of the appropriate size could be detected. A triple HA-tag was fused to the C-terminus of smORF18 (SEQ ID NO: 4) by PCR. First a PCR amplification was made using a primer corresponding to 400 bp upstream of smORF18 (L) and a second primer containing the C-terminus of smORF18 fused the HA-tag (5′-GGAGCCTGATCCAGCGTAGTCTGGGACGTCGTATGGGTAGCCAGCG TAGT CTGGGACGTCGTATGGGTAGCCAGCGTAATCCGGAACATCATACGG GTATCCTACGGCAGCAGCGGCAATAGGCTCAGG-3′) (SEQ ID NO:______). A second amplification was carried out with a forward primer containing the tag 5′-GTAGGATACCCGTATGATGTTCCGGATTACGCTGGCTACCCATA CGACGTCCCAGACTACGCTGGCTACCCATACGACGTCCCAGACTAC GCTGGATCAGGCTCCTAAAGATGAGAGGCTAGATCGAG-3′ (SEQ ID NO:______) and a primer located downstream of smORF18 (5′-TGTCGCTTTTTCTCCTCGATG AAGCCAAGCGCCGAACCAATTGATATCATCGGCACG-3′) (SEQ ID NO:______). The wild-type smORF18 gene was replaced with the tagged version by allele replacement into the chromosome (Erdeniz et al., 1997, Genome Res. 7: 1174). PCR amplification of the smORF18 (HA)₃ gene from genomic DNA followed by cloning and sequencing confirmed the identity of the tagged smORF18. For sequencing, PCR products were isolated from an agarose gel and then cloned in to pCR2.1-TOPO (Invitrogen). Soluble S100 extracts were prepared from diploid W303 (B. J. Thomas et al., 1989, Genetics 123:725) and from HA-tagged yeast cells grown in 25 ml of rich medium (YPD) to mid-log phase as described (Brown et al., 1996, Mol. Cell. Biol. 16: 5744). Soluble extracts were then fractionated in 18% polyacrylamide gels containing SDS. The proteins were then transferred to a PVDF membrane and the blot probed with anti-HA antibodies. The results show a protein band corresponding to a 9 kDa protein (FIG. 4, lanes 3 and 4) in extracts prepared from cells with a tagged smORF18 gene and not in wild-type cells. This result demonstrates that smORF18 (SEQ ID NO: 4) is not only transcribed, but also encodes a detectable protein product of the predicted size.

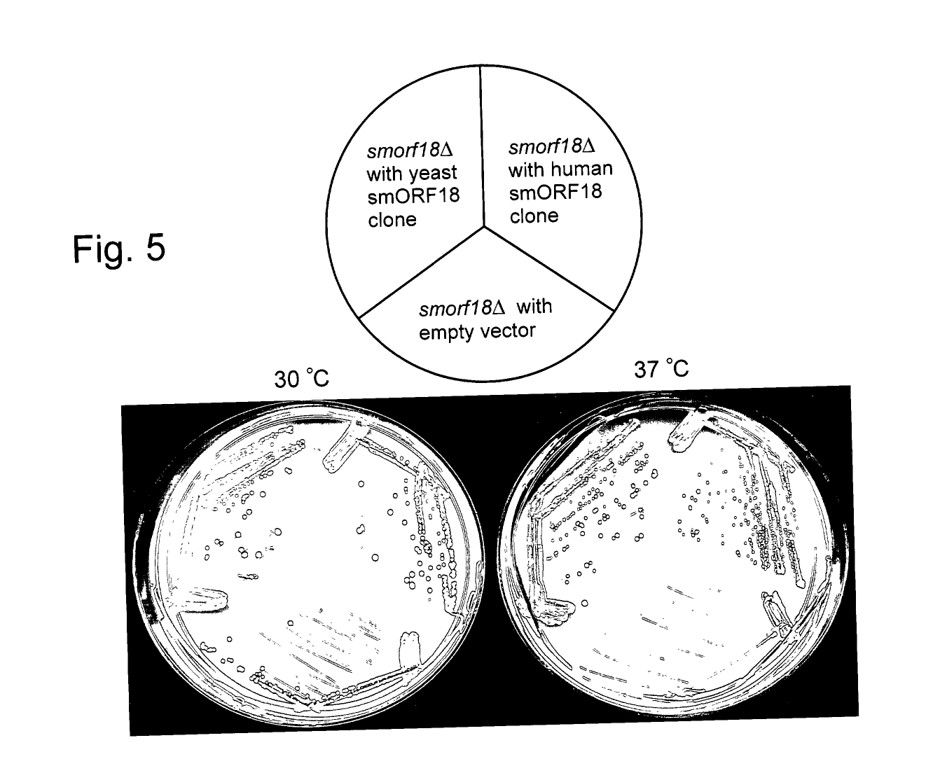

A next step of the process of identification and characterization of the gene is to further test if the smORF is essential. For example, one copy of the complete smORF18 gene was deleted in a diploid yeast strain by homologous recombination. Cells were transformed with a PCR fragment containing the HIS3 marker flanked by 400 bp of smORF18 sequences. The HIS3 sequence replaced amino acids 1 to 82 of smORF18. Histidine prototrophs were selected and PCR was used to verify correct genomic integration. Sporulation and tetrad analysis showed that haploid strains with a smorf18Δ were able to grow at 30° C. (slow growth), but not at 37° C. (FIG. 5). We next tested if the human smORF18 is a functional homolog of the yeast smORF18. The human smORF18 gene, which was obtained from an EST clone, and the yeast smORF18 were cloned into pYES (Invitrogen) vector for expression in yeast under the GAL1 promoter. The human smORF18 coding sequence was amplified from I.M.A.G.E. clone 1047404 (Research Genetics, Inc.). The yeast smORF18 was amplified from genomic DNA. PCR fragments were cloned into pYES2.1 /V5-His-TOPO (Invitrogen). Clones were verified by sequencing and transformed into the smorfΔ18strain. The resultant transformants were tested for the ability to complement the temperature sensitive phenotype of the smorf2Δ strain. The results demonstrate that the cloned human smORF18 as well as the yeast smORF18 (SEQ ID NO: 4) can complement the temperature sensitive phenotype of the smorf2Δ strain (FIG. 5). These results indicate that the human smORF18 is a functional ortholog of yeast smORF18 (SEQ ID NO: 4). The human smORF18 maps to two loci in the human genome, one in chromosome 3 where the gene contains two introns and codes for a predicted mRNA identical to the EST, and to a locus in chromosome 20 (i.e., 20g13.2-13.33, AL035669) without introns but with nine predicted amino acid substitutions. These data indicate that small ORFs are present and expressed in humans and underscores the importance of looking for small genes in the genomes of higher eukaryotes. smORF18 is essential for growth of yeast at 37 ° C. and has conserved homologs in organisms from yeast to man. smORF18 was used as bait in the two-hybrid analysis to isolate interactors. This gene is essential in yeast.

(iii) Yeast smORF139 (SEQ ID NO: 36). The smORF139 protein (SEQ ID NO: 709) appears to be a conserved protein in fungi. However, the conserved sequence, “LSGLQK”, is shared with lamin B2 from Xenopus laevis, chicken and human. The S. cerevisiae smORF139 protein is also 35% identical to an unidentified protein (AC003000) from Arabidopsis thaliana chromosome II (see below), and 33% identical to the middle section of glutathione transferase (S33628) from Dianthus caryophyllus (Clove pink). SigCleave (eGCG version 8) identified a weak signal peptide (score 0.9) from residue 13 to 26. No transmembrane domain was found. The A. fumigatus version has an intron in the gene. SmORF139 (SEQ ID NO: 709) was found in the region of ade2 gene for phosphoribosylaminoimidazole carboxylase, and pheromone response protein (RGA1) in Zygosaccharomyces rouxii. smORF139 (SEQ ID NO: 628) from S. cerevisiae is 74% identical to an unknown protein in Zygosaccharomyces rouxii. S. cerevisiae smORF139 also has a hit (38% identify) to a Medicago truncatula (plant) EST sequence (AW584424).

The smORF139 protein (SEQ ID NO: 709) is 35% identical to “Arabidopsis thaliana protein fragment SEQ ID NO: 1495” disclosed by Ceres Inc., on Feb. 25, 1999. The smORF139 is, however, conserved among fungi and therefore, could be used as a target for antifungal compositions described herein.

iv. Yeast smORF57. smORF57 (SEQ ID NO:13) is conserved between S. cerevisiae and C. albicans. The closest homolog in C. albicans is orf6.5842 and the following is the alignment between the two sequences:

Score = 94 (38.1 bits), Expect = 2.23−10, P = 2.2e−10
Identities = 23/89 (25%), Positives = 50/89 (56%)

| | | | |
|---|---|---|---|
| Sc: | 4 | NLSPLQQEVLDKYKQLSLDLKALDETIKELNYSQHRQQHSQQETVSPDEILQEMRDIEVK | 63 |
| | | NLSP++Q++L +Y+ ++ +L + ++ L + + ++ +++ +R +E K | |
| Ca: | 24 | NLSPIEQKILQQYQLMNNNLIKVSNELELLTNTTDEFGKGKGSSI---HLVENLRQLETK | 80 |
| Sc: | 64 | IGLVGTLLKGSVYSLILQRKQ--EQESLG 90 | |
| | | + V T KG+VYS++ + EQE+ G | |
| Ca: | 81 | LVFVYTFFKGAVYSILNAQDYIAEQETNG 109 | |

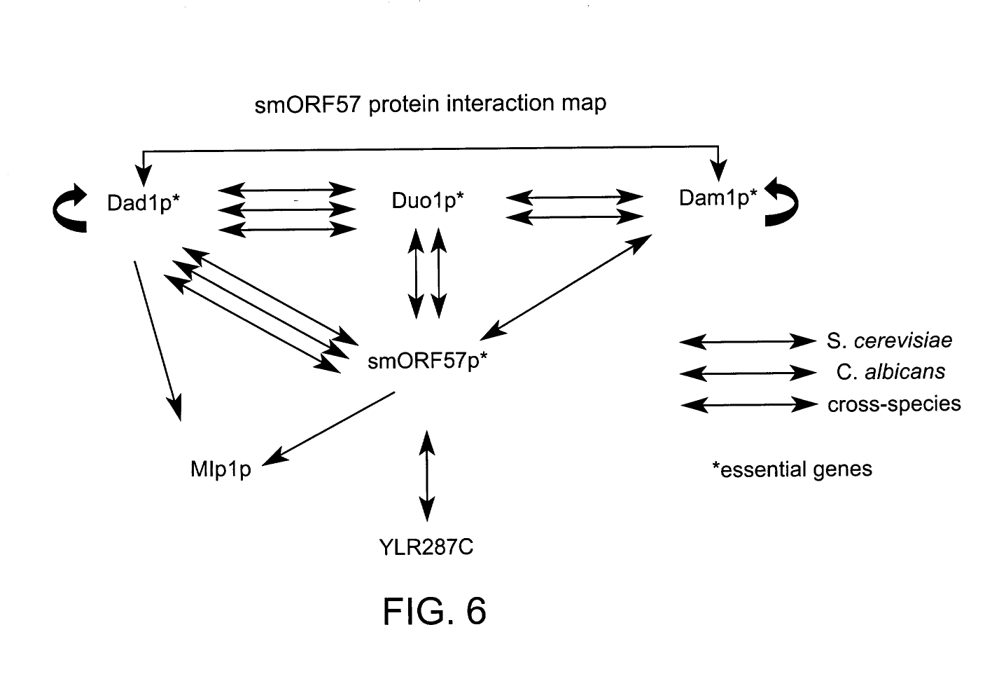

When smORF57 was used as bait three proteins were found as interactors, Dad1p, Dam1p, and Duo1p which are part of a complex of proteins that function in kinetochore function and are important for mitotic spindle. integrity. (Enquist-Newman M. et al., 2001 Mol. Biol. Cell. 12: 2601-2613). The interactions between smorf57 and Dad1p, Dam1p, and Duo1p have been confirmed by directed testing in the yeast two-hybrid system. Dam1p and Duo1p have homologs in C. albicans, which are orf6.7374 and orf6.6397 respectively. (Cheeseman I. M. et al. J. Cell. Biol. 152: 197-212). In addition, Dad1p has a homolog in C. albicans in Contig6-2505 (Enquist-Newman M., et al., 2001 Mol. Biol. Cell. 12: 2601-2613). The C. albicans genes coding for Dad1p, Dam1p, and Duo1p were also used in the yeast two-hybrid system to analyze the interactions. A diagram indicating the confirmed interactions between smORF57 and Dad1, Dam1, and Duo1 is shown in FIG. 6. smORF57 also interacted with Mlp1p, a non-essential (Myosin like protein 1) localized to the nucleus close to the nuclear envelope and the gene product from the YLR287C gene, which is a non-essential protein of unknown function.

The interaction of smORF57 with the Dad1/Dam1/Duo1 complex suggests that it also is involved in kinetochore function and mitotic spindle integrity. Moreover, the conservation of residues coupled with the lack of a human ortholog strongly suggests that smORF57 would be a target for antifungal treatment and compositions described herein. In addition, smORF57 would also be involved in diagnosing fungal infections which is also provided by this invention.

smORFs172 and 181 (SEQ ID NO: 43 and 44, respectively).

These two smORFs also have homologs in C. albicans and the alignments are shown below:

| | |
|---|---|
| smORF172 | (SEQID NO:43): |

Score = 339 (124.4 bits), Expect = 2.4e−30, P = 2.4e−30
Identities = 63/77 (81%), Positives = 69/77 (89%), Frame = −3

| | | | |
|---|---|---|---|
| Query: | 1 | MDALNSKEQQEFQKVVEQKQMKDFMRLYSNLVERCFTDCVNDFTTSKLTNKEQTCIMKCS | 60 |
| | | MD LN KEQQEFQ++VEQKQMKDFM LYSNLV RCF DCVNDFT++ LT+KE +CI KCS | |
| Sbjct: | 31134 | MDQLNVKEQQEFQQIVEQKQMKDFMNLYSMLVSRCFDDCVNDFTSNSLTSKETSCIAKCS | |
| Query: | 61 | EKFLKHSERVGQRFQEQ 77 | |
| | | EKFLKHSERVGQRFQEQ | |
| Sbjct: | 30954 | EKFLKHSERVGQRFQEQ 30904 | |

| | |
|---|---|
| smORF181 | (SEQ ID NO:44): |

Score = 192 (72.6 bits), Expect = 8.8e−15, P = 8.8e−15
Identities = 38/85 (44%), Positives = 56/85 (65%), Frame = +1

| | | | |
|---|---|---|---|
| Query: | 10 | RQVLSLYKEFIKNANQFNNYNFREYFLSKTRTTFRKNMNQQDPKVLMNLFKEAKNDLGVL | 69 |
| | | +Q+L LYK+ ++ A +F+NYNF+EY K TF+ N + + + + E N L +L | |
| Sbjct: | 4054 | KQILLLYKQLLEKAYKFDNYNFKEYSKRKIVETFKANKSLTNENEINQFYNEGINQLALL | 4233 |
| Query: | 70 | KRQSVISQMYTFDRLVVEPLQGRKH 94 | |
| | | RQ+ ISQ+YTFD+LVVEPL +KH | |
| Sbjct: | 4234 | YRQTTISQLYTFDKLVVEPL--KKH 4302 | |

The smORF172 (SEQ ID NO: 43) was recently annotated (TIM9) and its gene product is believed to be a translocase in the inner membrane of mitochondria involved in mitochondrial protein import. (Leuenberger D, et al. 1999. Different import pathways through the mitochondrial intermembrane space for inner membrane proteins. EMBO J 18: 4816-22).

The smORF181 is also conserved among fungal species thus implicating it as a target for antifungal treatment.

V. Additional smORF Validation.

To validate additional smORFs, the essentiality test was extended to 125 smORFs (Table 4) with the following results:

TABLE 4

| | SEQ ID | SmORF | |
|---|---|---|---|
| SEQ ID | NO | No. | Essentiality Result |
| SC0013 | 13 | smorf057 | Confirmed essential |
| SC0034 | 34 | smorf127 | Possibly essential |
| SC0043 | 43 | smorf172 | Confirmed essential |
| SC0044 | 44 | smorf181 | Confirmed essential |
| SC0047 | 47 | smorf207 | Possibly essential |
| SC0052 | 52 | smorf268 | Possibly essential |
| SC0060 | 60 | smorf303 | Possibly essential |
| SC0068 | 68 | smorf337 | Possibly essential |
| SC0089 | 89 | smorf532 | Possibly essential |
| SC0104 | 104 | smorf601 | Possibly essential |
| SC0108 | 108 | smorf626 | Possibly essential |
| SC0111 | 111 | smorf640 | Possibly essential |
| SC0184 | 184 | smorf117 | Possibly essential |
| SC0190 | 190 | smorf136 | Possibly essential |
| SC0329 | 329 | smorf330 | Possibly essential |
| SC0334 | 334 | smorf335 | Possibly essential |
| SC0654 | 654 | smorf520 | Possibly essential |
| SC0572 | 572 | smorf639 | Possibly essential |
| SC0562 | 562 | smorf623 | Possibly essential |

Three smORFs were determined to be essential (SEQ ID NO: 13, 43 and 44). Sixteen other sequences, which are listed in Table 4, were determined to encode possibly essential proteins. The remaining sequences of the 125 analyzed were determined as non-essential. The C. albicans presumptive homolog of smORF57 (orf6.5842) was also disrupted with the result that it is essential. In addition, sixteen S. cerevisiae smORFs are potential essential, but essentiality needs to be confirmed by gene disruption in the diploid strain followed by sporulation and tetrad analysis (SEQ ID NO: 34, 47, 52, 60, 68, 89, 104, 108, 111, 184, 190, 329, 334, 654, 572, and 562). The remaining smORFs were non-essential (Table 4).

IV. Pharmaceutical Compositions

Once essential genes are identified, compounds and compositions can be screened for their ability to modulate the activity of the gene. For example, agents can be screen for C. albicans essential genes to determine whether the compound has antifungal properties. Essential genes of C. albicans, for example, that do not have plant and/or mammalian homologs can be used as targets for the design and discovery of highly specific antifungal agents. Also preferred would be the identification of essential fungal and bacterial genes that have insect or plant homologs. Compounds and compositions that target such genes could be used as insecticides and herbicides. In another embodiment, essential genes which have mammalian homologs can be used as targets for the design of anti-proliferative agents or agents which inhibit proliferation or progression of the organism and/or its associated disease process.

Candidate agents which can be used to screen and eventually to treat conditions and diseases associated with the organisms, such as C. albicans encompass numerous chemical classes, though typically they are organic molecules, preferably small organic molecules having a molecular weight of more than 100 and less than about 2,500 Daltons. Candidate agents are obtained from a wide variety of sources including libraries of synthetic or natural compounds. They can include peptides, macromolecules, small molecules, chemical and/or biological mixtures, and fungal, bacterial, or algal extracts. Such compounds, or molecules, may be biological, synthetic, organic, or even inorganic compounds, and may be obtained from several sources, including pharmaceutical companies and specialty suppliers of libraries (e.g., combinatorial libraries) of compounds. Libraries can also include peptide libraries.

Methods of the present invention are well suited for screening libraries of compounds in multiwell plates (e.g., 96-, 384-, or higher density well plates), with a different test compound in each well. In particular, the methods may be employed with combinatorial libraries. A variety of combinatorial libraries of random-sequence oligonucleotides, polypeptides, or synthetic oligomers have been proposed. A number of small-molecule libraries have also been developed.

Combinatorial libraries may be formed by a variety of solution-phase or solid-phase methods in which mixtures of different subunits are added step-wise to growing oligomers or parent compounds, until a desired compound is synthesized. A library of increasing complexity can be formed in this manner, for example, by pooling multiple choices of reagents with each additional subunit step. Methods of preparing combinatorial libraries the use of microwaving, dynamic combinatorial chemistry (DCC), solid phase organic synthesis (SPOS), and dual recursive deconvolution (DRED) as example. See, e.g., Borman, “Combinatorial Chemistry”, Chem. Eng. News 49-58 (Aug. 27, 2001).

The identity of library compounds with desired effects on the target protein can be determined by conventional means, such as iterative synthesis methods in which sublibraries containing known residues in one subunit position only are identified as containing active compounds.

Preferred compounds may have characteristics of IC₅₀ values between about 15 and about 50 μM; preferably a low mammalian cellular toxicity (e.g., GI₅₀>100 μM). In the example of C. albicans, preferable compounds will have antifungal activity of at least about 3-50 μM against C. albicans, as well was other fungal agents associated with disease. Preferred antifungal agents will be those that are fungicidal, e.g., which cause the selective death of the fungus. Preferred antibiotics will cause the death of the fungal organism without detrimentally (e.g., causing cell death in the host organism infected by the fungus) affecting the condition of the host organism infected by the fungal organism.

Generally, the preferred compositions and methods provided herein are directed at preventing and treating infections caused by but not limited to Chytridiomycetes, Hyphochrytridiomycetes, Plasmodiophoromycetes, Oomycetes, Zygomycetes, Ascomycetes, and Basidiomycetes. Fungal infections which can be inhibited or treated with compositions provided herein include but are not limited to: Candidiasis including but not limited to onchomycosis, chronic mucocutaneous candidiasis, oral candidiasis, epiglottistis, esophagitis, gastrointestinal infections, genitourinary infections, for example, caused by any Candida species, including but not limited to Candida albicans, Candida tropicalis, Candida (Torulopsis) glabrata, Candida parapsilosis, Candida lusitaneae, Candida rugosa and Candida pseudotropicalis; Aspergillosis including but not limited to granulocytopenia caused for example, by, Aspergillus spp. including but not limited to A. fumigatus, Aspergillus flavus, Aspergillus niger and Aspergillus terreuis; Zygomycosis, including but not limited to pulmonary, sinus and rhinocerebral infections caused by, for example, zygomycetes such as Mucor. Rhizopus spp., Absidia, Rhizomucor, Cuiningamella, Saksenaea, Basidobolus and Conidobolus; Cryptococcosis, including but not limited to infections of the central nervous system—meningitis and infections of the respiratory tract caused by, for example, Cryptococcus neoformans; Trichosporonosis caused by, for example, Trichosporon beigelii; Pseudallescheriasis caused by, for example, Pseudallescheria boydii; Fusarium infection caused by, for example, Fusarium such as Fusarium solani, Fusarium moniliforme and Fusarium proliferatum; and other infections such as those caused by, for example, Penicillium spp. (generalized subcutaneous abscesses), Drechslera, Bipolaris, Exserohilum spp., Paecilomyces lilacinum, Exophila jeanselmei (cutaneous nodules), Malassezia furfur (folliculitis), Alternaria (cutaneous nodular lesions), Aureobasidium pullulans (splenic and disseminated infection), Rhodotorula spp. (disseminated infection), Chaetomium spp. (empyema), Torulopsis candida (fungemia), Curvularia spp. (nasopharnygeal infection), Cunninghamella spp. (pneumonia), H. Capsulatum, B. dermatitidis, Coccidioides immitis, Sporothrix schenckii and Paracoccidioides brasiliensis, Geotrichum candidum (disseminated infection).

Treating “fungal infections” as used herein refers to the treatment of conditions resulting from fungal infections. Therefore, contemplated is the treatment of, for example, pneumonia, nasopharnygeal infections, disseminated infections and other conditions listed above and known in the art by using the compositions provided herein. In preferred embodiments, treatments and sanitization of areas with the compositions provided herein can be used to treat immuno-compromised patients or areas where there are such patients. Wherein it is desired to identify the particular fungi resulting in the infection, techniques known in the art may be used.

One of skill in the art will readily appreciate that the methods described herein also can be used for diagnostic applications. A diagnostic as used herein is a compound or method that assists in the identification and characterization of a health or disease state in humans or other animals, by a product of a gene identified by a disclosed method. The use of the genes and gene products thus identified are useful tools in vitro for fungal infection determination.

V. Antisense Compositions and Use Thereof

In another embodiment, antisense compounds, compositions and methods are provided for modulating the expression of genes identified by the above-described methods. Preferable antisense compounds are those which target nucleic acids identified using a systematic in silico discovery method disclosed herein. Preferred antisense compounds can target, for example, SEQ ID NOS: 1-119 (See Table 2). Of those, most preferred are agents that target essential genes such as smORF57 (SEQ ID NO: 13).

It is preferred to target specific nucleic acids for antisense. “Targeting” an antisense compound to a particular nucleic acid would preferably be to a nucleic acid that encodes a protein, wherein the nucleic acid is one identified by a systematic in silico process disclosed herein. The gene can be from a pathogenic organism. The targeting includes determination of a site or sites within the target gene for the antisense reaction (e.g., joinder of the sense and antisense strands to thereby modulate function of the gene or gene transcript). Preferred antisense compounds are those that recognize and bind with a site encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes).

It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding a protein which was identified by a systematic in silico method disclosed herein or one of the sequences disclosed herein.

A translation termination codon (or “stop codon”) of a gene's transcript may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Preferred antisense compositions would recognize and bind to areas containing a termination codon and/or an initiation codon of any target gene or the mRNA transcript it encodes.

The open reading frame (ORF) or “coding region,” which is known in the art to refer to the region between the translation initiation codon and the translation termination codon, is also a region which may be preferred targets of the antisense compounds or compositions. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′→5′ triphosphate linkage. The 5′ cap region of an mRNA is considered to include the 5′ cap structure itself, and the first 50 nucleotides adjacent to the cap. The 5′ cap region may also be a preferred target region for an antisense compound or composition.

In the instance of more complex eukaryotic organisms, the genes are composed of introns and exons, with the exons containing the material that will encode the protein product of the gene. The intronic material, although transcribed from the gene to produce the mRNA, will be excised from the mRNA transcript prior to its translation into a protein. The exons are spliced together to form a continuous mRNA sequence. The mRNA splice sites, i.e., intron-exon junctions, may also be preferred target regions of antisense compounds and compositions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites are identified in the genes identified using a systematic discovery process disclosed herein, oligonucleotides are chosen which are sufficiently complementary to the target, i.e., hybridize sufficiently well and with sufficient specificity, to result produce the desired biological outcome (e.g., inhibition of microorganism proliferation or progression, inhibition and/or prevention of the disease or condition induced by the microorganism, modulation of the activity of the targeted gene).

In the context of this invention, “hybridization” means hydrogen bonding, which may be Watson-Crick, Hoogsteen or reversed Hoogsteen hydrogen bonding, between complementary nucleoside or nucleotide bases. For example, adenine (A) and thymine (T) are complementary nucleobases, which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound or composition to non-target sequences under conditions in which specific binding is desired. Preferred conditions for specific binding are physiological conditions in the case of in vivo assays or therapeutic treatment, and in the case of in vitro assays, under conditions in which the assays are performed.

Preferred antisense compounds and compositions contemplated would be for use as research reagents and diagnostics. For example, antisense oligonucleotides, which are able to inhibit gene expression, are often used by those of ordinary skill to elucidate the function of particular genes. Antisense compounds and compositions are also used, e.g., to distinguish between functions of various members of a biological pathway. Antisense modulation has, therefore, been harnessed for research use.

Oligonucleotides have been employed as therapeutic moieties in the treatment of disease states in animals and man. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, e.g., enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked nucleosides). The antisense compounds can be longer than 30 (e.g., 35, 40, 45, 50, 55, 60, 65, 70, 75, 80, 85, 90, 95, 100 or more as well as ranges in between). However, more preferred antisense compounds are comprise from about 12 to about 25 nucleobases.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure. However, open linear structures are generally preferred for use as antisense compounds or in antisense compositions. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones for use in antisense compounds and compositions include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. For additional deals in preparing such phosphorus containing linkages, see for example, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones that do not include a phosphorus atom may have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. For methods of preparing modified oligonucleotide backbones that lack phosphorous atoms, see, e.g., U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other preferred oligonucleotide mimetics include replacement of both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. For discussion of such methods, see for example, U.S. Pat. Nos. 5,539,082; 5,714,331; and 5,719,262 and Nielsen et al., Science, 1991, 254: 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] and amide backbones such as those described in U.S. Pat. No. 5,602,240. Also preferred are oligonucleotides having morpholino backbone structures, such as those described in U.S. Pat. No. 5,034,506.

Modified oligonucleotides used as antisense compounds or in antisense compositions as contemplated herein may also contain one or more substituted sugar moieties. Preferred oligonucleotides comprise one of the following at the 2′ position: —OH; F—; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH_{2,} and O(CH₂)ₙON[(CH₂)ₙCH₃)]₂, where n and m are from 1 to about 10. Other preferred oligonucleotides may comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH_{2,} heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂—CH₂—OCH₃, also known as 2′-O-(2-methoxyethyl) or 2′-MOE) (Martin et al., Helv. Chim. Acta, 1995, 78: 486-504), i.e., an alkoxyalkoxy group. Another preferred modification includes 2′-dimethylaminooxyethoxy (i.e., a O(CH₂)₂ ON(CH₃)₂ group, also known as 2′-DMAOE) and 2′-dimethylaminoethoxyethoxy (also known in the art as 2′-O-dimethylaminoethoxyethyl or 2′-DMAEOE).

Other preferred modifications to the antisense compounds contemplated include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly at the 3′ position of the sugar on the 3′ terminal nucleotide or in 2′-5′ linked oligonucleotides and the 5′ position of 5′ terminal nucleotide. Oligonucleotides may also have sugar mimetics, such as cyclobutyl moieties in place of the pentofuranosyl sugar. For methods of preparing such modified sugar structures, see for example, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). The invention also contemplates the use of modified nucleobases in the antisense compounds and compositions. Such modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (e.g., particularly 5-bromo, 5-trifluoromethyl) and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional nucleobases would be known to the skilled artisan. See for example, U.S. Pat. No. 3,687,808; THE CONCISE ENCYCLOPEDIA OF POLYMER SCIENCE AND ENGINEERING, 858-859 (Kroschwitz, J. I., ed. John Wiley & Sons, 1990); Englisch et al., ANGEWANDTE CHEMIE, v.30, p. 613 (International Edition, 1991); and Sanghvi, Y. S., Chapter 15, ANTISENSE RESEARCH AND APPLICATIONS, 289-302 (Crooke et al., CRC Press, 1993). Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., et al., 1993) and are presently preferred base substitutions, even more particularly when combined with 2′-O-methoxyethyl sugar modifications.

Another oligonucleotide modification contemplated for use in the antisense compounds and compositions involves chemically linking to the oligonucleotide one or more moieties or conjugates that enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86: 6553-6), cholic acid (Manoharan et al., Bioorg. Med. Chem. Lett., 1994, 4: 1053-60), a thioether, e.g., hexyl-S-tritylthiol (Manoharan et al., Ann. N.Y. Acad. Sci., 1992, 660: 306-9; and Manoharan et al., Bioorg. Med. Chem. Lett., 1993, 3: 2765-70), a thiocholesterol (Oberhauser et al., Nucl. Acids Res., 1992, 20: 533-8), an aliphatic chain, e.g., dodecandiol or undecyl residues (Saison-Behmoaras et al., EMBO J., 1991, 10: 1111-8; Kabanov et al., FEBS Lett., 1990, 259: 327-30; and Svinarchuk et al., Biochimie, 1993, 75: 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36: 3651-4; and Shea et al., Nucl. Acids Res., 1990, 18: 3777-83), a polyamine or a polyethylene glycol chain (Manoharan et al., Nucleosides & Nucleotides, 1995, 14: 969-73), or adamantane acetic acid (Manoharan et al., Tetrahedron Lett., 1995, 36: 3651-4), a palmityl moiety (Mishra et al., Biochim. Biophys. Acta, 1995, 1264: 229-237), or an octadecylamine or hexylamino-carbonyl-oxycholesterol moiety (Crooke et al., J. Pharmacol. Exp. Ther., 1996, 277: 923-937).

Methods for preparing such oligonucleotide conjugates would be known in the art and include but are not limited to U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

One or more of the positions in a given compound can be modified. It is not necessary for all positions in a given compound to be uniformly modified, and in fact more than one of the aforementioned modifications may be incorporated in a single compound or even at a single nucleoside within an oligonucleotide.

The present invention also includes antisense compounds that are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have are also known as hybrids or gapmers. Methods of preparing such hybrids include but are not limited to the teachings of U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922.

The antisense compounds contemplated herein may be conveniently and routinely made through the well-known technique of solid phase synthesis. The oligonucleotides can be prepared for example using the equipment and techniques of Applied Biosystems. Any other means for such synthesis known in the art may additionally or alternatively be employed.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Methods and preparations for such uptake, distribution and/or absorption assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The contemplated antisense compounds and compositions disclosed herein also include any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed for example in WO 93/24510 and in WO 94/26764.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. The compounds for modulating any of the disclosed genes, gene transcripts or proteins encoded thereby include antisense compounds as well as other modulatory compounds.

Pharmaceutically acceptable base addition salts for use with antisense as well as other modulatory compounds are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, e.g., Berge et al., “Pharmaceutical Salts,” J. Pharma. Sci., 1977, 66: 1-19). The base addition salts of acidic compounds are prepared by contacting the free acid form with a sufficient amount of the desired base to produce the salt in the conventional manner. The free acid form may be regenerated by contacting the salt form with an acid, and isolating the free acid in a conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are known in the art and include basic salts of a variety of inorganic and organic acids, such as, for example, with inorganic acids (e.g., hydrochloric acid, hydrobromic acid, sulfuric acid or phosphoric acid); with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid.

Pharmaceutically acceptable salts of compounds may also be prepared with a pharmaceutically acceptable cation. Suitable pharmaceutically acceptable cations are well known in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds and other modulatory compounds described herein can be utilized in pharmaceutical compositions by adding an effective amount of an antisense compound or other modulatory compound to a suitable pharmaceutically acceptable diluent or carrier. Use of the compounds and methods of the invention may also be useful prophylactically, e.g., to prevent or delay infection, progression of the microorganism, or inflammation, for example.

The antisense compounds of the invention are useful for research and diagnostics, because these compounds hybridize to nucleic acids encoding a gene identified using the systematic discovery technique or an mRNA transcript thereof. Such hybridization allows the use of sandwich and other assays to easily be constructed to exploit this fact. Hybridization of the antisense oligonucleotides of the invention with a nucleic acid encoding a gene or gene transcript identified by a systematic discover method can be detected by means known in the art. Such means may include conjugation of an enzyme to the oligonucleotide, radiolabelling of the oligonucleotide or any other suitable detection means. Kits using such detection means for detecting the level of a transcript of a gene in a sample may also be prepared.

The present invention also includes pharmaceutical compositions and formulations that include the antisense compounds and other modulatory compounds and compositions of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer), intratracheal, intranasal, epidermal and transdermal, oral or parenteral. Parenteral administration includes intravenous (i.v.), intraarterial, subcutaneous (s.c.), intraperitoneal (i.p.) or intramuscular (i.m.) injection or - infusion; or intracranial (e.g., intrathecal or intraventricular) administration. Oligonucleotides with at least one 2′-O-methoxyethyl modification are believed to be particularly useful for oral administration

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions (e.g., gene, gene transcript or protein product modulatory agents as described herein) of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention, the pharmaceutical compositions may be formulated and used as foams. Pharmaceutical foams include formulations such as, but not limited to, emulsions, microemulsions, creams, jellies and liposomes. While basically similar in nature, these formulations vary in the components and the consistency of the final product. The preparation of such compositions and formulations is generally known to those skilled in the pharmaceutical and formulation arts and may be applied to the formulation of the compositions of the present invention.

The compositions of the present invention may be prepared and formulated as emulsions. Emulsions are typically heterogenous systems of one liquid dispersed in another in the form of droplets usually exceeding 0.1 μm in diameter. See, e.g., Idson, in PHARMACEUTICAL DOSAGE FORMS v. 1, p. 199 (Lieberman, Rieger and Banker (Eds.), 1988, Marcel Dekker, Inc., New York); Rosoff, in PHARMACEUTICAL DOSAGE FORMS, v. 1,p. 245; Block in PHARMACEUTICAL DOSAGE FORMS, v. 2, p. 335; Higuchi et al., in REMINGTON'S PHARMACEUTICAL SCIENCES 301 (Mack Publishing Co., Easton, Pa., 1985). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug that may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in PHARMACEUTICAL DOSAGE FORMS v. 1, p. 199 (Lieberman, Rieger and Banker (Eds.), 1988, Marcel Dekker, Inc., New York).

Synthetic surfactants, also known as surface active agents, have found wide applicability in the formulation of emulsions and have been reviewed in the literature (Rieger, in PHARMACEUTICAL DOSAGE FORMS,v. 1, p. 285; Idson, in PHARMACEUTICAL DOSAGE FORMS, v. 1,p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in PHARMACEUTICAL DOSAGE FORMS).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers, especially in combination with surfactants and in viscous preparations. These include polar inorganic solids, such as heavy metal hydroxides, non-swelling clays (e.g., bentonite, attapulgite, hectorite, kaolin, montmorillonite, colloidal aluminum silicate and colloidal magnesium aluminum silicate), pigments and nonpolar solids (e.g., carbon or glyceryl tristearate).

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in PHARMACEUTICAL DOSAGE FORMS, v.1 p.385 (Lieberman, Rieger and Banker (Eds.), 1988, Marcel Dekker, Inc., New York)).

Hydrophilic colloids or hydrocolloids include naturally occurring gums and synthetic polymers, such as polysaccharides (e.g., acacia, agar, alginic acid, carrageenan, guar gum, karaya gum, and tragacanth), cellulose derivatives (e.g., carboxymethylcellulose and carboxypropylcellulose), and synthetic polymers (e.g., carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers (e.g., tocopherols, alkyl gallates, butylated hydroxyanisole, butylated hydroxytoluene) or reducing agents (e.g., ascorbic acid and sodium metabisulfite), and antioxidant synergists (e.g., citric acid, tartaric acid, and lecithin).

The application of emulsion formulations via dermatological, oral and parenteral routes and methods for their manufacture have been reviewed in the literature (Idson, in PHARMACEUTICAL DOSAGE FORMS, v. 1, p. 199). Emulsion formulations for oral delivery have been very widely used because of reasons of ease of formulation, efficacy from an absorption and bioavailability standpoint. (Rosoff, in PHARMACEUTICAL DOSAGE FORMS, v. 1, p. 245 (Lieberman, Rieger and Banker (Eds.), 1988, Marcel Dekker, Inc., New York); Idson, in PHARMACEUTICAL DOSAGE FORMS). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in PHARMACEUTICAL DOSAGE FORMS, v. 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in CONTROLLED RELEASE OF DRUGS: POLYMERS AND AGGREGATE SYSTEMS, 185-215 (Rosoff, M., Ed., 1989, VCH Publishers, New York). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in REMINGTON'S PHARMACEUTICAL SCIENCES, 271 (Mack Publishing Co., Easton, Pa., 1985).

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with co-surfactants. The co-surfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Microemulsions may, however, be prepared without the use of co-surfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono-, di-, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharm. Res., 1994, 11:1385-90; Ritschel, Meth. Find. Exp. Clin. Pharmacol., 1993, 13: 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., 1994; Ho et al., J. Pharm. Sci., 1996, 85: 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids and other active agents from the gastrointestinal tract, as well as improve the local cellular uptake of oligonucleotides and nucleic acids and other active agents within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Crit. Rev. Therap. Drug Carrier Systems, 1991, p. 92). Each of these classes has been discussed above.

There are many organized surfactant structures besides microemulsions that have been studied and used for the formulation of drugs. These include monolayers, micelles, bilayers and vesicles. Vesicles, such as liposomes, are useful because of their specificity and the duration of action. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. Selection of the appropriate liposome depending on the agent to be encapsulated would be evident given what is known in the art.

In order to cross mammalian skin, lipid vesicles must pass through a series of fine pores, each with a diameter less than 50 nm, under the influence of a suitable transdermal gradient. Therefore, it is desirable to use a liposome that is highly deformable and able to pass through such fine pores.

Further advantages of liposomes include: (a) liposomes obtained from natural phospholipids are biocompatible and biodegradable; (b) liposomes can incorporate a wide range of water and lipid soluble drugs; (c) liposomes can protect encapsulated drugs in their internal compartments from metabolism and degradation (Rosoff, in PHARMACEUTICAL DOSAGE FORMS). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Another embodiment also contemplates the use of liposomes for topical administration. Such advantages include reduced side-effects related to high systemic absorption of the administered drug, increased accumulation of the administered drug at the desired target, and the ability to administer a wide variety of drugs, both hydrophilic and hydrophobic, into the skin. Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes that interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Comm., 1987, 147:, 980-5).

Liposomes that are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., J. Controlled Release, 1992, 19: 269-74).

Another contemplated liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

“Sterically stabilized” liposomes that refer to liposomes comprising one or more specialized lipids that, when incorporated into liposomes, result in enhanced circulation lifetimes relative to liposomes lacking such specialized lipids are also contemplated. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Lett., 1987, 223: 42; Wu et al., Can. Res., 1993, 53: 3765).

Many liposomes comprising lipids derivatized with one or more hydrophilic polymers, and methods of preparation thereof, are known in the art. See, e.g., Sunamoto et al. (Bull. Chem. Soc. Jpn., 1980, 53: 2778) described liposomes comprising a nonionic detergent, 2C₁₂ 15G, that contains a PEG moiety. Illum et al. (FEBS Lett., 1984, 167: 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268: 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029: 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by, e.g., Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.). Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

Methods of encapsulating nucleic acids in liposomes are also known in the art. See, WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in PHARMACEUTICAL DOSAGE FORMS, p.285 (Marcel Dekker, Inc., New York, N.Y., 1988, p. 285)).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in PHARMACEUTICAL DOSAGE FORMS, 285 (Marcel Dekker, Inc., New York, N.Y., 1988).

In one embodiment, the present invention employs various penetration enhancers to affect the efficient delivery of nucleic acids and other agents, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p.92). Each of the above mentioned classes of penetration enhancers are described below in greater detail.

Another embodiment of the invention contemplates pharmaceutical compositions comprising surfactants. Surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Crit. Rev. Therap. Drug Carrier Systems, 1991, 92); and perfluorochemical emulsions, such as FC-43 (Takahashi et al., J. Pharm. Pharmacol., 1988, 40: 252).

Another embodiment contemplates the use of various fatty acids and their derivatives to act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, and the like) (Lee et al., 1991; Muranishi, Crit. Rev. Therap. Drug Carrier Systems, 1990, 7: 1-33; El Hariri et al., J. Pharm. Pharmacol., 1992, 44: 651-4).

The compositions comprising the active agents of the invention may further comprise bile salts. The physiological role of bile includes the facilitation of dispersion and absorption of lipids and fat-soluble vitamins (Brunton, Chapter 38 in: GOODMAN & GILMAN'S THE PHARMACOLOGICAL BASIS OF THERAPEUTICS, 9th Ed., Hardman et al. Eds., McGraw-Hill, N.Y., 1996, pp. 934-935). Various natural bile salts, and their synthetic derivatives, act as penetration enhancers. Thus, the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., 1991; Swinyard, Chapter 39 In: REMINGTON'S PHARMACEUTICAL SCIENCES, 18th Ed., Gennaro, ed., Mack Publishing Co., Easton, Pa., 1990, pages 782-783; Muranishi, 1990; Yamamoto et al., J. Pharm. Exp. Ther., 1992, 263: 25; Yamashita et al., J. Pharm. Sci., 1990, 79: 579-83).

The invention further contemplates compositions comprising chelating agents. Chelating agents can be defined as compounds that remove metallic ions from solution by forming complexes therewith, with the result that absorption of oligonucleotides through the mucosa is enhanced. With regards to their use as penetration enhancers for use when the active agent is an antisense agent, chelating agents have the added advantage of also serving as DNase inhibitors, as most characterized DNA nucleases require a divalent metal ion for catalysis and are thus inhibited by chelating agents (Jarrett, J. Chromatogr., 1993, 618: 315-39). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., 1991; Muranishi, 1990; Buur et al., J. Control Rel., 1990, 14: 43-51).

The invention also contemplates pharmaceutical compositions comprising active agents and non-chelating non-surfactants. Non-chelating non-surfactant penetration enhancing compounds can be defined as compounds that demonstrate insignificant activity as chelating agents or as surfactants, but that nonetheless enhance absorption of oligonucleotides through the alimentary mucosa (Muranishi, 1990). This class of penetration enhancers include, for example, unsaturated cyclic ureas, 1-alkyl- and 1-alkenylazacyclo-alkanone derivatives (Lee et al., 1991); and non-steroidal anti-inflammatory agents such as diclofenac sodium, indomethacin and phenylbutazone (Yamashita et al., J. Pharm. Pharmacol., 1987, 39: 621-6).

For pharmaceutical compositions comprising oligonucleotides, agents that enhance uptake of oligonucleotides at the cellular level may also be added to the pharmaceutical and other compositions of the present invention. For example, cationic lipids, such as lipofectin (Junichi et al., U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5: 115-121; Takakura et al., Antisense & Nucl. Acid Drug Dev., 1996, 6: 177-183).

The pharmaceutical compositions disclosed herein may also comprise one or more pharmaceutically acceptable excipients. In contrast to carrier compounds described above, these excipients include a pharmaceutically acceptable solvent, suspending agent or any other pharmacologically inert vehicle for delivering one or more nucleic acids or other active agents to an animal. The excipient may be liquid or solid and is selected, with the planned manner of administration in mind, so as to provide for the desired bulk, consistency, etc., when combined with a nucleic acid or other active agent and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients suitable for non-parenteral administration, which do not deleteriously react with nucleic acids, can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids and other contemplated active agents may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration that do not deleteriously react with nucleic acids or other contemplated active agents can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, e.g., antipruritics, astringents, local anesthetics or anti- inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances that increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds, and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, THE MERCK MANUAL OF DIAGNOSIS AND THERAPY, 1206-28 (15th Ed., Berkow et al., eds., 1987, Rahway, N.J.). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, THE MERCK MANUAL OF DIAGNOSIS AND THERAPY, 2499-2506 and 46-49 (15th Ed., Berkow et al., eds., 1987, Rahway, N.J.) respectively. Other non-antisense chemotherapeutic agents are also within the scope of this invention. Two or more combined compounds may be used together or sequentially.

In another related embodiment, compositions of the invention may contain one or more antisense compound or other active agents. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on ECs found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

VI. Polypeptide and Peptides

The polypeptides or peptides of the invention are isolated polypeptides or peptides. Preferably these polypeptides are encoded by the smORF identified by the in silico process, but they can also be prepared synthetically or by a recombinant nucleic acid which would encode the same protein, but is different due to code degeneracy than the smORF sequence identified in silico.

As used herein, with respect to peptides, the term “isolated peptides” and “isolated polypeptides” and “isolated protein” mean that the compounds are substantially pure and are essentially free of other substances with which they may be found in nature or in vivo systems to an extent practical and appropriate for their intended use. In particular, the compounds are sufficiently pure and are sufficiently free from other biological constituents of their hosts' cells so as to be useful in, for example, producing pharmaceutical preparations or sequencing. Because an isolated peptide (which as used herein also includes polypeptides and proteins) of the invention may be admixed with a pharmaceutically acceptable carrier in a pharmaceutical preparation, the peptide may comprise only a small percentage by weight of the preparation. The peptide is nonetheless substantially pure in that it has been substantially separated from the substances with which it may be associated in living systems.

The polypeptides and proteins of the invention can be used to prepare antibodies, to identify ligand binding partners, in competition assays, and the like as would be known in the art. These assays using fragments of the proteins may be based on motifs identified in the polypeptides, such as the representative examples shown in Table 3 (Motifs).

VII. Antibodies Antibody Fragments and Immunologically Active Immunogens

The invention also contemplates preparation and use of immunoglobulins against the proteins encoded by the smORFs. By immunoglobulins is meant to include antibodies, antibody fragments (e.g., Fab, Fab′, Fv, scFv, and F(ab)₂), bispecific antibodies, polyclonal and monoclonal antibodies, human and humanized antibodies, bivalent antibodies and antibody fragments and the like.

A. Humanized and Primatized® Antibodies

The invention further provides humanized immunoglobulins (or antibodies). The humanized antibodies are preferably specific to the protein encoded by a specific smORF. These humanized and primatized® antibodies are useful as therapeutic and diagnostic reagents in their own right or can be combined to form a humanized or primatized® bispecific antibody possessing both of the binding specificities of its components.

The humanized and primatized® forms of immunoglobulins have variable framework region(s) substantially from a human immunoglobulin (termed an acceptor immunoglobulin) and complementarity determining regions substantially from a mouse immunoglobulin (referred to as the donor immunoglobulin). The constant region(s), if present, are also substantially from a human immunoglobulin. The humanized antibodies exhibit a specific binding affinity for their respective antigens of at least 10⁷, 10⁸, 10^{9,} or 10¹⁰ M⁻¹. Often the upper and lower limits of binding affinity of the humanized antibodies are within a factor of three or five or ten of that of the mouse (or other animal) antibody from which they were derived.

A “humanized monoclonal antibody” as used herein is a human monoclonal antibody or functionally active fragment thereof having human constant regions and a region that binds to a protein encoded by a smORF, wherein that region is from a mammal of a species other than a human. Humanized monoclonal antibodies may be made by any method known in the art. A “primatized® monoclonal antibody” would be one having a domain from a primate, such as a cynomolgus macaque. For example, see Anderson et al., 1997, Clin. Immunol. Immunopathol. 84: 73-84and U.S. Pat. Nos. 6,001,358 and 6,113,898.

Humanized monoclonal antibodies, for example, may be constructed by replacing the non-CDR regions of a non-human mammalian antibody with similar regions of human antibodies while retaining the epitopic specificity of the original antibody. For example, non-human CDRs and optionally some of the framework regions may be covalently joined to human FR and/or Fc/pFc′ regions to produce a functional antibody. Certain corporations are now humanizing antibodies from specific murine antibody regions, e.g., Protein Design Labs (Mountain View Calif.).

European Patent Application 0 239 400 provides an exemplary teaching of the production and use of humanized monoclonal antibodies in which at least the complementarity determining regions (CDR) portion of a murine (or other non-human mammal) antibody is included in the humanized antibody. Briefly, the following methods are useful for constructing a humanized CDR monoclonal antibody including at least a portion of a mouse CDR. A first replicable expression vector including a suitable promoter operably linked to a DNA sequence encoding at least a variable domain of an Ig heavy or light chain and the variable domain comprising framework regions from a human antibody and a CDR region of a murine antibody is prepared. Optionally a second replicable expression vector is prepared which includes a suitable promoter operably linked to a DNA sequence encoding at least the variable domain of a complementary human Ig light or heavy chain respectively. A cell line is then transformed with the vectors. Preferably the cell line is an immortalized mammalian cell line of lymphoid origin, such as a myeloma cell line, or is a normal lymphoid cell that has been immortalized by transformation with a virus. The transformed cell line is then cultured under conditions known to those of skill in the art to produce the humanized antibody.

As set forth in European Patent Application 0 239 400, several techniques are well known in the art for creating the particular antibody domains to be inserted into the replicable vector. For example, the DNA sequence encoding the domain may be prepared by oligonucleotide synthesis. Alternatively a synthetic gene lacking the CDR regions in which four framework regions are fused together with suitable restriction sites at the junctions, such that double stranded synthetic or restricted subcloned CDR cassettes with sticky ends could be ligated at the junctions of the framework regions. Another method involves the preparation of the DNA sequence encoding the variable CDR containing domain by oligonucleotide site-directed mutagenesis. Each of these methods is well known in the art. Therefore, those skilled in the art may construct humanized antibodies containing a murine CDR region without destroying the specificity of the antibody for its epitope.

As noted above, such humanized antibodies may be produced in which some or all of the FR regions of deposited monoclonal antibody have been replaced by homologous human FR regions. In addition, the Fc portions may be replaced so as to produce IgA or IgM as well as human IgG antibodies bearing some or all of the CDRs of the deposited monoclonal antibody. In a more preferred embodiment, a murine CDR is grafted into the framework region of a human antibody to prepare the “humanized antibody.” See, e.g., L. Riechmann et al., 1988, Nature 332: 323; M. S. Neuberger et al., 1985 Nature 314: 268; and EPA 0 239 400 (published Sep. 30, 1987).

In one embodiment of the invention, the peptide containing a region that binds to a polypeptide encoded by a smORF is a functionally active antibody fragment. Significantly, as is well known in the art, only a small portion of an antibody molecule, the paratope, is involved in the binding of the antibody to its epitope (see, in general, Clark, W. R. (1986) THE EXPERIMENTAL FOUNDATIONS OF MODERN IMMUNOLOGY Wiley & Sons, Inc., New York; Roitt, I. (1991) ESSENTIAL IMMUNOLOGY, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, designated a Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment may be associated with up to ten different light chains without altering antibody specificity) and Fd fragments retain epitope-binding ability in isolation. Another preferred fragment is the scFv fragment.

(i) Mouse Antibodies for Humanization. The starting material for production of humanized antibody specific could be a protein or immunlogically active portion thereof encoded by SEQ ID NOS: 674-1346 or polypeptides identified by the disclosed in silico methods.

(ii) Selection of Human Antibodies to Supply Framework Residues. The substitution of mouse CDRs into a human variable domain framework is most likely to result in retention of their correct spatial orientation if the human variable domain framework adopts the same or similar conformation to the mouse variable framework from which the CDRs originated. This is achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies.

Suitable human antibody sequences are identified by computer comparisons of the amino acid sequences of the mouse variable regions with the sequences of known human antibodies. The comparison is performed separately for heavy and light chains but the principles are similar for each.

(iii) Computer Modeling. The unnatural juxtaposition of murine (or other animal) CDR regions with human variable framework region can result in unnatural conformational restraints, which, unless corrected by substitution of certain amino acid residues, lead to loss of binding affinity. The selection of amino acid residues for substitution is determined, in part, by computer modeling. Computer hardware and software for producing three-dimensional images of immunoglobulin molecules are widely available. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

Computer modeling can also be utilized to identify the portions of a protein encoded by a smORF that has a good antigenic profile or hydrophobicity profile. This can be performed using algorithms set up by Chou-Fasman and the GOR method (Chou et al., 1978, Adv. Enzymol. Relat. Areas Mol. Biol. 47: 45-147; and Gamier et al., 1978, J. Mol. Biol. 120: 97-120). The proteins can also be analyzed using various available computer algorithms to determine whether the potential antigenic region is buried within the protein or is exposed at the surface of the protein. See, e.g., David W. Mount, BIOINFORMATICS: SEQUENCE AND GENOME ANALYSIS 381-478 (Cold Spring Harbor Laboratory Press, 2001). Alternatively, the antibodies and fragments thereof can be prepared to bind to domains identified by protein modeling, such as those of Table 3 (Motifs).

(iv) Substitution of Amino Acid Residues. As noted supra, the humanized antibodies of the invention comprise variable framework region(s) substantially from a human immunoglobulin and complementarity determining regions substantially from a mouse immunoglobulin. Having identified the complementarity determining regions of mouse antibodies and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human anti-murine antibody (HAMA) response in humans. Amino acids are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

When an amino acid differs between a mouse variable framework region and an equivalent human variable framework region, the human framework amino acid should usually be substituted by the equivalent mouse amino acid if it is reasonably expected that the amino acid:

(1) noncovalently contacts antigen directly, or

(2) is adjacent to a CDR region or otherwise interacts with a CDR region (e.g., is within about 4-6 Å of a CDR region). Other candidates for substitution are acceptor human framework amino acids that are unusual for a human immunoglobulin at that position. These amino acids can be substituted with amino acids from the equivalent position of more typical human immunoglobulins. Alternatively, amino acids from equivalent positions in the mouse antibody can be introduced into the human framework regions when such amino acids are typical of human immunoglobulin at the equivalent positions.

In general, substitution of all or most of the amino acids fulfilling the above criteria is desirable. Occasionally, however, there is some ambiguity about whether a particular amino acid meets the above criteria, and alternative variant immunoglobulins are produced, one of which has that particular substitution, the other of which does not.

Usually the CDR regions in humanized antibodies are substantially identical, and more usually, identical to the corresponding CDR regions in the mouse antibody from which they were derived. Although not usually desirable, it is sometimes possible to make one or more conservative amino acid substitutions of CDR residues without appreciably affecting the binding affinity of the resulting humanized immunoglobulin. Occasionally, substitutions of CDR regions can enhance binding affinity.

Other than for the specific amino acid substitutions discussed above, the framework regions of humanized immunoglobulins are usually substantially identical, and more usually, identical to the framework regions of the human antibodies from which they were derived. Of course, many of the amino acids in the framework region make little or no direct contribution to the specificity or affinity of an antibody. Thus, many individual conservative substitutions of framework residues can be tolerated without appreciable change of the specificity or affinity of the resulting humanized immunoglobulin.

(v) Production of Variable Regions. Having conceptually selected the CDR and framework components of humanized immunoglobulins, a variety of methods are available for producing such immunoglobulins. Because of the degeneracy of the code, a variety of nucleic acid sequences will encode each immunoglobulin amino acid sequence. The desired nucleic acid sequences can be produced by de novo solid-phase DNA synthesis or by PCR mutagenesis of an earlier prepared variant of the desired polynucleotide. All nucleic acids encoding the antibodies described in this application are expressly included in the invention.

(vi) Selection of Constant Region. The variable segments of humanized antibodies produced as described supra are typically linked to at least a portion of an immunoglobulin constant region (Fc), typically that of a human immunoglobulin. Human constant region DNA sequences can be isolated in accordance with well-known procedures from a variety of human cells, but preferably immortalized B-cells (see, e.g., WO87/02671). Ordinarily, the antibody will contain both light chain and heavy chain constant regions. The heavy chain constant region usually includes C_{H}1, hinge, C_{H}2, C_{H}3, and, sometimes, C_{H}4 regions.

The humanized antibodies include antibodies having all types of constant regions, including IgM, IgG, IgD, IgA and IgE, and any isotype, including IgG1, IgG2, IgG3 and IgG4. When it is desired that the humanized antibody exhibit cytotoxic activity, the constant domain is usually a complement-fixing constant domain and the class is typically IgG1. When such cytotoxic activity is not desirable, the constant domain may be of the IgG2 class. The humanized antibody may comprise sequences from more than one class or isotype.

(vii) Expression Systems. Nucleic acids encoding humanized light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Such control sequences include a signal sequence, a promoter, an enhancer, and a transcription termination sequence (see Queen et al., 1989, Proc. Natl. Acad. Sci. USA 86: 10029; WO 90/07861; Co et al., 1992, J. Immunol. 148: 1149).

B. Fragments of Humanized Antibodies

The humanized antibodies of the invention include fragments as well as intact antibodies. Typically, these fragments compete with the intact antibody from which they were derived for antigen binding. The fragments typically bind with an affinity of at least 10⁷ M⁻¹, and more typically 10⁸ or 10⁹ M⁻¹ (i.e., within the same ranges as the intact antibody). Humanized antibody fragments include separate heavy chains, light chains Fab, Fab′, F(ab′)₂, Fv, and scFv. Fragments are produced by recombinant DNA techniques, or by enzymatic or chemical separation of intact immunoglobulins.

C. Recombinant Bispecific Antibodies

The methods discussed above for forming bispecific antibodies from antibodies produced by hybridoma cells can also be applied or adapted to production of bispecific antibodies from recombinantly expressed antibodies. For example, bispecific antibodies can be produced by fusion of two cell lines respectively expressing the component antibodies. Alternatively, the component antibodies can be co-expressed in the same cell line. Bispecific antibodies can also be formed by chemical cross-linking of component recombinant antibodies.

Component recombinant antibodies can also be linked genetically. In one approach, a bispecific antibody is expressed as a single fusion protein comprising the four different variable domains from the two component antibodies separated by spacers. For example, such a protein might comprise from one terminus to the other, the V_{L} region of the first component antibody, a spacer, the V_{H} domain of the first component antibody, a second spacer, the V_{H} domain of the second component antibody, a third spacer, and the V_{L} domain of the second component antibody. See, e.g., Segal et al., 1992 Biologic Therapy of Cancer Updates 2: 1-12.

In a further approach, bispecific antibodies are formed by linking component antibodies to leucine zipper peptides. See generally Kostelny et al., 1992, J. Immunol. 148: 1547-1553. Leucine zippers have the general structural formula (Leucine-X₁ -X₂ -X₃ -X₄ -X₅ -X₆)ₙ, where X may be any of the conventional 20 amino acids (PROTEINS, STRUCTURES AND MOLECULAR PRINCIPLES, (1984) Creighton (ed.), W. H. Freeman and Company, New York), but are most likely to be amino acids with high α-helix forming potential. For example, alanine, valine, aspartic acid, glutamic acid, and lysine (Richardson et al., 1988, Science 240: 1648), and n may be 3 or greater, although typically n is 4 or 5.

In the formation of bispecific antibodies, binding fragments of the component antibodies are fused in-frame to first and second leucine zippers. Suitable binding fragments including Fv, Fab, Fab′, or the heavy chain. The zippers can be linked to the heavy or light chain of the antibody binding fragment and are usually linked to the C-terminal end. If a constant region or a portion of a constant region is present, the leucine zipper is preferably linked to the constant region or portion thereof. For example, in a Fab′-leucine zipper fusion, the zipper is usually fused to the C-terminal end of the hinge. The inclusion of leucine zippers fused to the respective component antibody fragments promotes formation of heterodimeric fragments by annealing of the zippers. When the component antibodies include portions of constant regions (e.g., Fab′ fragments), the annealing of zippers also serves to bring the constant regions into proximity, thereby promoting bonding of constant regions (e.g., in a F(ab′)₂ fragment). Typical human constant regions bond by the formation of two disulfide bonds between hinge regions of the respective chains. This bonding can be strengthened by engineering additional cysteine residue(s) into the respective hinge regions, which allows formation of additional disulfide bonds.

Leucine zippers linked to antibody binding fragments can be produced in various ways. For example, polynucleotide sequences encoding a fusion protein comprising a leucine zipper can be expressed by a cellular host or by using an in vitro translation system. Alternatively, leucine zippers and/or antibody binding fragments can be produced separately, either by chemical peptide synthesis, by expression of polynucleotide sequences encoding the desired polypeptides, or by cleavage from other proteins containing leucine zippers, antibodies, or macromolecular species, and subsequent purification. Such purified polypeptides can be linked by peptide bonds, with or without intervening spacer amino acid sequences, or by non-peptide covalent bonds, with or without intervening spacer molecules, the spacer molecules being either amino acids or other non-amino acid chemical structures. Regardless of the method or type of linkage, such linkage can be reversible. For example, a chemically labile bond, either peptidyl or otherwise, can be cleaved spontaneously or upon treatment with heat, electromagnetic radiation, proteases, or chemical agents. Two examples of such reversible linkage are: (1) a linkage comprising an Asn-Gly peptide bond which can be cleaved by hydroxylamine, and (2) a disulfide bond linkage which can be cleaved by reducing agents.

Component antibody fragment-leucine zippers fusion proteins can be annealed by co-expressing both fusion proteins in the same cell line. Alternatively, the fusion proteins can be expressed in separate cell lines and mixed in vitro. If the component antibody fragments include portions of a constant region (e.g., Fab′ fragments), the leucine zippers can be cleaved after annealing has occurred. The component antibodies remain linked in the bispecific antibody via the constant regions.

As used herein the term “functionally active antibody fragment” means a fragment of an antibody molecule including a region that binds to a protein or fragment thereof encoded by a smORF, wherein the antibody fragment retains the T-cell stimulating functionality of an intact antibody having the same specificity such as the deposited monoclonal antibodies. Such fragments are also well known in the art and are regularly employed both in vitro and in vivo. In particular, well-known functionally active antibody fragments include but are not limited to F(ab′)₂, Fab, Fv, scFv and Fd fragments of antibodies. These fragments that lack the Fc fragment of intact antibody, clear more rapidly from the circulation, and may have less non-specific tissue binding than an intact antibody. For example, single-chain antibodies can be constructed in accordance with the methods described in U.S. Pat. No. 4,946,778 to Ladner et al. Such single-chain antibodies include the variable regions of the light and heavy chains joined by a flexible linker moiety. Methods for obtaining a single domain antibody (“Fd”) which comprises an isolated variable heavy chain single domain, also have been reported (see, for example, Ward et al., 1989, Nature 341: 644-646, disclosing a method of screening to identify an antibody heavy chain variable region (V_{H} single domain antibody) with sufficient affinity for its target epitope to bind thereto in isolated form). Methods for making recombinant Fv fragments based on known antibody heavy chain and light chain variable region sequences are known in the art and have been described, e.g., U.S. Pat. No. 4,462,334. Other references describing the use and generation of antibody fragments include e.g., Fab fragments (Tijssen, PRACTICE AND THEORY OF ENZYME IMMUNOASSAYS (Elsevieer, Amsterdam, 1985)), Fv fragments (Hochman et al., 1973 Biochemistry 12: 1130; Sharon et al., 1976 Biochemistry 15: 1591; Ehrilch et al., U.S. Pat. No. 4,355,023) and portions of antibody molecules (e.g., Audilore-Hargreaves, U.S. Pat. No. 4,470,925).

Functionally active antibody fragments also encompass “humanized antibody fragments.” As one skilled in the art will recognize, such fragments could be prepared by traditional enzymatic cleavage of intact humanized antibodies. If, however, intact antibodies are not susceptible to such cleavage, because of the nature of the construction involved, the noted constructions can be prepared with immunoglobulin fragments used as the starting materials; or, if recombinant techniques are used, the DNA sequences, themselves, can be tailored to encode the desired “fragment” which, when expressed, can be combined in vivo or in vitro, by chemical or biological means, to prepare the final desired intact immunoglobulin fragment.

Smaller antibody fragments and small binding polypeptides having binding specificity are also contemplated. Several routine assays may be used to easily identify such peptides. Screening assays for identifying peptides of the invention are performed for example, using phage display procedures such as those described in Hart et al., 1994, J. Biol. Chem. 269: 12468. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to a smORF polypeptide are obtained by selecting those phages, which express on their surface a ligand that binds to the smORF polypeptide. These phages then are subjected to several cycles of reselection to identify the peptide ligand-expressing phages that have the most useful binding characteristics. Typically, phages that exhibit the best binding characteristics (e.g., highest affinity) are further characterized by nucleic acid analysis to identify the particular amino acid sequences of the peptides expressed on the phage surface and the optimum length of the expressed peptide to achieve optimum binding to the protein or polypeptide fragment encoded by a smORF. Alternatively, such peptide ligands can be selected from combinatorial libraries of peptides containing one or more amino acids. Such libraries can further be synthesized which contain non-peptide synthetic moieties, which are less subject to enzymatic degradation compared to their naturally occurring counterparts.

Additionally small polypeptides including those containing the smORF polypeptide binding CDR3 region may easily be synthesized or produced by recombinant means to produce the peptide of the invention. Such methods are well known to those of ordinary skill in the art. Peptides can be synthesized for example, using automated peptide synthesizers, which are commercially available. The peptides can be produced by recombinant techniques by incorporating the DNA expressing the peptide into an expression vector and transforming cells with the expression vector to produce the peptide.

The sequence of the CDR regions, for use in synthesizing the peptides of the invention, may be determined by methods known in the art. The heavy chain variable region is a peptide, which generally ranges from 100 to 150 amino acids in length (or any number in between). The light chain variable region is a peptide, which generally ranges from 80 to 130 amino acids in length (or any number in between). The CDR sequences within the heavy and light chain variable regions, which include only approximately 3-25 amino acid sequences (including any number in between), may easily be sequenced by one of ordinary skill in the art. The peptides may even be synthesized synthetically by commercial sources such as by the Scripps Protein and Nucleic Acids Core Sequencing Facility (La Jolla Calif.).

To determine whether a peptide binds to a smORF polypeptide, any known binding assay may be employed. For example, the peptide may be immobilized on a surface and then contacted with a labeled smORF polypeptide. The amount of smORF polypeptide that interacts with the peptide or the amount that does not bind to the peptide may then be quantitated to determine whether the peptide binds to the smORF polypeptide. A surface having the deposited monoclonal antibody immobilized thereto may serve as a positive control.

Screening of peptides of the invention, also can be carried out utilizing a competition assay. If the peptide being tested competes with the deposited monoclonal antibody, as shown by a decrease in binding of the deposited monoclonal antibody, then it is likely that the peptide and the deposited monoclonal antibody bind to the same, or a closely related, epitope. Still another way to determine whether a peptide has the specificity of, for example a monoclonal antibody, is to pre-incubate the deposited monoclonal antibody with the smORF polypeptide with which it is normally reactive, and then add the peptide being tested to determine if the peptide being tested is inhibited in its ability to bind to the smORF polypeptide. If the peptide being tested is inhibited then, in all likelihood, it has the same, or a functionally equivalent, epitope and specificity as the deposited monoclonal antibody. Other methods and assays would be evident to the artisan of ordinary skill.

D. Therapeutic Methods

Pharmaceutical compositions comprising bispecific antibodies of the present invention are useful for parenteral administration, i.e., subcutaneously (s.c.), intramuscularly (I.M.) and particularly, intravenously (I.V.). Other contemplated forms of administration, depending on the particular need, would be oral, intrathecal, and intraperitoneal. The compositions for parenteral administration commonly comprise a solution of the antibody or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of particulate matter. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate. The concentration of the bispecific antibodies in these formulations can vary widely, i.e., from less than about 0.01%, usually at least about 0.1% to as much as 5% by weight and will be selected primarily based on fluid volumes, and viscosities in accordance with the particular mode of administration selected.

A typical antibody or antibody fragment composition for intravenous infusion can be made up to contain, for example, 250 ml of sterile Ringer's solution, and 10 mg of bispecific antibody. See REMINGTON'S PHARMACEUTICAL SCIENCE (15th Ed., Mack Publishing Company, Easton, Pa., 1980).

The compositions containing the antibodies or antibody cocktails or a cocktail thereof can be administered for prophylactic and/or therapeutic treatments. In therapeutic application, compositions are administered to a subject with a fungal infection, which expresses a smORF polypeptide of interest. The amount administered to the patient is sufficient to cure or ameliorate the infection or corresponding condition caused by the fungus. An amount adequate to accomplish this is defined as a “therapeutically effective dose.” Amounts effective for use with antibodies or antibody fragments will depend upon the severity of the condition and the general state of the subject, but generally range from about 0.01 to about 100 mg of antibody per dose, with dosages of from 0.1 to 50 mg and 1 to 10 mg per patient being more commonly used. Single or multiple administrations on a daily, weekly or monthly schedule can be carried out with dose levels and pattern being selected by the treating physician.

In prophylactic applications, compositions containing the antibodies, fragments or peptides which bind to smORF polypeptides or a cocktail thereof are administered to a patient who is at risk of developing the disease state to enhance the patient's resistance. Such an amount is defined to be a “prophylactically effective dose.” In this use, the precise amounts again depend upon the subject's state of health and general level of immunity, but generally range from 0.1 to 100 mg per dose, especially 1 to 10 mg per patient.

E. Diagnostic Methods

The antibodies and antibody fragments and peptides that bind to smORF polypeptides can also be useful in diagnostic methods for diagnosing fungal infections. Methods of diagnosis can be performed in vitro using a cellular sample (e.g., blood sample, lymph node biopsy or tissue) from a patient and performing a histological analysis of the sample, or can be performed by in vivo imaging. These methods are readily known in the art.

While the present invention has been described with specificity in accordance with certain of its preferred embodiments, the examples discussed herein serve only to illustrate the invention and are not intended to limit the same.

F. Vaccines

For smORFs identified using the methods described herein, the proteins encoded by these smORFs may be determined to be useful for the preparation of vaccines. Typically, proteins, or antigenic fragments thereof, are chosen based on their exposure on the surface of a virus, cell or organism, thus exposing them to the immune cells of a host. Additionally, these proteins and protein fragments must be antigenic or immunogenic (i.e. the ability of a substance to act as an antigen, which elicits a specific immune response when introduced into a host.

The pharmaceutical compositions for use in obtaining an immune response would contain such pharmaceutical excipients, adjuvants and/or carriers as are standard in preparations designed to obtain an immune response. The therapeutic response would be one wherein the subject to which the pharmaceutical composition was administered would have a protective effect (i.e., preventing the subject from contracting an infection due to the microorganism for which the subject had been treated).

(i) Selection of Immunogen. Vaccines against fungal organisms are important to the treatment of a variety of diseases and conditions. For example, Cryptococcus neoformans is an opportunistic fungal pathogen which causes an incurable, life-threatening meningoencephalitis in patient populations with AIDS. Coccidioidomycosis is another emerging health problem in light of the increasing numbers of immunosuppressed patients. Most infections are caused by Coccidioides immitis, which can advance into coccidioidal pneumonia or extrapulmonary infection. Thus, vaccines against these and other funguses is becoming more important, especially with increasing numbers of immune compromised individuals.

Selection of immunogen can be based on one or more factors such as (1) cell surface exposure and availability of the protein to a host immune cell, (2) predicted antigenicity/immunogenicity of the immunogen, (3) whether the immunogen may be N- or O-linked glycosylated; and (4) an extracellular protein (e.g., proteinases, esterases and lipases). Certain glyocosylated proteins have served as good antigens in raising an immune response in animals such as MP98 of Cryptococcus neoformans in mice (Levitz et al., Proc. Natl. Acad. Sci. USA 98: 10422-27, 2001); MP65 mannoprotein of Candida albicans (Antonio, Nippon Ishinkin Gakkai Zasshi 41: 219, 2000) and the cryptococcal capsular glucuronoxylomannan protected against systemic mycosis in mice (Devi, Vaccine 14: 1298, 1996). Heat shock proteins have also been identified as suitable candidates for antifungal vaccines (Deepe et al, J. Immunol. 167: 2219-26, 2001).

(ii) Polypeptide and DNA Vaccines. Antifungal vaccines can be prepared in a variety of ways. For purposes of this invention, living and non-living (i.e., derived from the entire microorganism) fungal vaccines are less preferred. More preferred are vaccine formulations that can be administered as (1) polypeptides, (2) polypeptides conjugated to another antigenic compound, (3) direct inoculation of plasmid DNA encoding the desired smORF, wherein expression is driven by a strong promoter capable of efficient activity in a variety of mammalian cell types.

Once suitable immunogens are identified, protein based vaccines can prepared wherein one or more smORF polypeptides (20-500 μg polypeptide, more preferably about 50-150 μg ) are mixed with a pharmaceutically acceptable adjuvant. If testing in animals, an injection is administered to the animal, followed by second and third injections a few weeks later. For example, 100 μg of polypeptide (or combination of polypeptides) is admixed with a desired adjuvant (e.g., Ribi adjuvant, RIBI ImmunoChem Research Inc.). The material can be injected intramuscularly or subcutaneously in an animal subject. In mice, the protectiveness of the vaccine can be measured by footpad hypersensitivity testing. For instance, the peptide is prepared and injected into the hind footpads of the mice with either 50 μl of spherule-phase smORF polypeptide diluted in non-pyrogenic saline or in saline alone. Footpad thickness is then measured with a dual caliper and the results calculated as the difference in footpad thickness of antigen- and saline-injected pads at 18 to 25 hours minus the difference in footpad thickness of antigen- and saline injected pads before challenge. Lack of footpad sensitivity indicates that the mice have received some protective immunity with the injected antigen.

Additional methods for preparing, using and assaying pharmaceutical compositions for inducing a protective immune response can be performed according to what is known in the art. See, for example S. H. E. Kaufmann, Concepts in Vaccine Development (Walter De Gruyter 1996); Devi, Vaccine 14: 841-4 (1996); Deepe et al., J. Immunol. 167: 2219-26 (2001) and Levitz et al., Proc. Natl. Acad. Sci. USA 98: 10422-27 (2001).

For purposes of conferring immunogenicity using a DNA vaccine, the plasmid containing and operably linked to the desired smORF would be administered, for example as follows. The desired smORF would be operably linked into a plasmid, such as pGEX-4-T3 (Pharmaceia Biotech, Piscataway, N.J.) downstream from the gene encoding glutathione S-transferase (GST). The smORF containing plasmid is then amplified and preferably purified. The plasmid can then be immunized in mice or other suitable animal. If using mice, (for example in an assay system), the mice are injected with 200 μl of the smORF containing plasmid (100 μg) or the plasmid alone (100 μg). The plasmid is in a mixture with saline and admixed with an equal volume of Ribi adjuvant (RIBI ImmunoChem Research, Inc.) or other DNA vaccine suitable adjuvant. Additional components may be present such as synthetic trehalose dicorynomycolate (TDM) and cell wall skeleton. The DNA containing composition is typically administered intramuscularly or subcutaneously. Second or third injects can also be given via intramuscular or subcutaneous routes. The plasmid can also be administered intraperitoneally (i.p.). See, e.g., Jiang et al., “Genetic Vaccination against Coccidioides immitis: Comparison of Vaccine Efficacy of Recombinant Antigen 2 and Antigen 2 cDNA,” Infection & Immun. 67: 630-5 (1999).

In vivo assays of animals, such as mice, can be performed to determine the protectiveness of a particular smORF or smORFs or antigenic fragments thereof. Once animals have been injected with the smORF DNA, as discussed above, the animals can be challenged with exposure to the particular microorganism. Typically challenge is by intraperitoneal injection of the microorganism into the animal and assessment of survival of the mice with the vaccine as compared to control animals. See, e.g., Jiang et al., “Genetic Vaccination against Coccidioides immitis: Comparison of Vaccine Efficacy of Recombinant Antigen 2 and Antigen 2 cDNA,” Infection & Immun. 67: 630-5 (1999). Additional methods of preparing, administering, and assaying such compositions would be apparent to the artisan. See for example, “Development and Clinical Progress of DNA Vaccines: Paul-Ehrlich-Institut” in Developments in Biologicals vol. 104 (F. Brown et al., eds. S. Karger Publ., 2000); “DNA Vaccines: Methods and Protocols” in Methods in Molecular Medicine vol. 29 (Douglas B. Lowrie and Robert G. Whalen eds, Humana Press, 2000); Yvonne Paterson, Intracellular Bacterial Vaccine Vectors: Immunology Cell Biology and Genetics (Wiley-Liss, 1999); Bruce H. Nicholson, Synthetic Vaccines (Blackwell Science Inc. 1994); and Richard E. Isaacson, Recombinant DNA Vaccines (Marcel Dekker, 1992).

All references discussed above are herein incorporated by reference in their entirety.

TABLE 2

| | NT | AA | NT ORF | AA ORF | | | |
|---|---|---|---|---|---|---|---|
| smorf | Seq ID | Seq ID | Length | Length | Score | Probability | Description |
| smorf003 | 1 | 674 | 195 | 64 | 68 | 0.038 | gp:[GI:1334567] [LN:MTPACG] [AC:X55026:M30937:M61734] |
| | | | | | | | [PN:Dod ND1 i4 grp IB protein a] [GN:ND1] [OR:Mitochondrion |
| | | | | | | | Podospora anserina] [SR:Podospora anserina] [DB:genpept-pln3] |
| | | | | | | | [DE:Podospora anserina complete mitochondrial genome.] |
| | | | | | | | [LE:<97174] [RE:98349] [DI:direct] |
| smorf013 | 2 | 675 | 297 | 98 | 179 | 3.1E−12 | pir:[LN:T38980] [AC:T38980] [PN: protein SPAC630.02] |
| | | | | | | | [GN:SPAC630.02] [OR:Schizosaccharomyces pombe] [DB:pir2] |
| | | | | | | | [MP:1]>gp:[GI:5734463] [LN:SPAC630] [AC:AL109832] [PN: |
| | | | | | | | Protein involved in cell shape and cell] [GN:SPAC630.02] |
| | | | | | | | [OR:Schizosaccharomyces pombe] [SR:fission yeast] [DB:genpept- |
| | | | | | | | pln4] [DE:S.pombe chromosome I cosmid c630.] [NT:SPAC630.02, |
| | | | | | | | len:905, SIMILARITY:Saccharomyces] [LE:1577] [RE:4294] |
| | | | | | | | [DI:direct] |
| smorf016 | 3 | 676 | 606 | 201 | 510 | 1.3E−48 | pir:[LN:S78703] [AC:S78703] [PN:protein YBL091c-a] |
| | | | | | | | [OR:Saccharomyces cerevisiae] [DB:pir2] [MP:2L] |
| smorf018 | 4 | 677 | 282 | 93 | 222 | 4.4E−18 | pir:[LN:T39177] [AC:T39177] [PN: protein SPAC8F11.02c] |
| | | | | | | | [GN:SPAC8F11.02c] [OR:Schizosaccharomyces pombe] [DB:pir2] |
| | | | | | | | [MP:1]>gp:[GI:5701971] [LN:SPAC8F11] [AC:AL109738] [PN: |
| | | | | | | | protein; low similarity to DNAJ] [GN:SPAC8F11.02c] |
| | | | | | | | [OR:Schizosaccharomyces pombe] [SR:fission yeast] [DB:genpept- |
| | | | | | | | pln4] [DE:S.pombe chromosome I cosmid c8F11.] |
| | | | | | | | [NT:SPAC8F11.02c, len:79, SIMILARITY:Caenorhabditis] |
| | | | | | | | [LE:1881:2075:2179] [RE:2015:2136:2221] [DI:complement Join] |
| smorf019 | 5 | 678 | 318 | 105 | 579 | 6.5E−56 | sp:[LN:AST1_YEAST] [AC:P35183] |
| | | | | | | | [GN:AST1:YBL069W:YBL0617:YBL06.04] [OR:Saccharomyces |
| | | | | | | | cerevisiae] [SR:,Baker's yeast] [DE:AST1 PROTEIN] [SP:P35183] |
| | | | | | | | [DB:swissprot]>gp:[GI:551276] [LN:SCAST1] [AC:X81843] |
| | | | | | | | [GN:AST1] [OR:Saccharomyces cerevisiae] [SR:baker's yeast] |
| | | | | | | | [DB:genpept-pln4] [DE:S.cerevisiae AST1 gene.] [SP:P35183] |
| | | | | | | | [LE:415] [RE:1704] [DI:direct]>gp:[GI:1870081] [LN:SCYBL070C] |
| | | | | | | | [AC:Z35831:Y13134] [GN:AST1] [OR:Saccharomyces cerevisiae] |
| | | | | | | | [SR:baker's yeast] [DB:genpept-pln4] [DE:S.cerevisiae chromosome |
| | | | | | | | II reading frame ORF YBL070c.] [NT:ORF YBL069w] [SP:P35183] |
| | | | | | | | [LE:210] [RE:1499] [DI:direct] |
| smorf024 | 6 | 679 | 252 | 83 | | | |
| smorf028 | 7 | 680 | 186 | 61 | 318 | 2.9E−28 | gp:[GI:4388567] [LN:SCYBR007C] [AC:Z35876:Y13134] |
| | | | | | | | [OR:Saccharomyces cerevisiae] [SR:baker's yeast] [DB:genpept- |
| | | | | | | | pln4] [DE:S.cerevisiae chromosome II reading frame ORF |
| | | | | | | | YBR007c.] [NT:ORF YBR006w] [LE:<1] [RE:189] [DI:direct] |
| smorf032 | 8 | 681 | 252 | 83 | 423 | 2.2E−39 | pir:[LN:S78706] [AC:S78706] [PN:protein YBR058c-a] |
| | | | | | | | [OR:Saccharomyces cerevisiae] [DB:pir2] [MP:2R] |
| smorf044 | 9 | 682 | 228 | 75 | | | |
| smorf046 | 10 | 683 | 312 | 103 | 73 | 0.032 | pir:[LN:S20693] [AC:S20693] [PN: protein 12.3 K (early region E3)] |
| | | | | | | | [CL:adenovirus early E3B 14.5 K protein] [OR:Mastadenovirus h41] |
| | | | | | | | [SR:,human adenovirus 41] [DB:pir2]>gp:[GI:303998] |
| | | | | | | | [LN:ADRGENOME] [AC:L19443] [OR:Human adenovirus type 40] |
| smorf053 | 11 | 684 | 231 | 76 | | | |
| smorf054 | 12 | 685 | 183 | 60 | | | |
| smorf057 | 13 | 686 | 330 | 109 | 84 | 0.012 | pir:[LN:B71661] [AC:B71661] [PN: protein RP564] [GN:RP564] |
| | | | | | | | [OR:Rickettsia prowazekii] [DB:pir2]>gp:[GI:3861112] [LN:RPXX03] |
| | | | | | | | [AC:AJ235272:AJ235269] [PN:] [GN:RP564] [OR:Rickettsia |
| | | | | | | | prowazekii] [DB:genpept-bct3] [DE:Rickettsia prowazekii strain |
| | | | | | | | Madrid E, complete genome; segment3/4.] [LE:112399] [RE:113382] |
| | | | | | | | [DI:complement] |
| smorf066 | 14 | 687 | 654 | 217 | 1103 | 1.9E−111 | sp:[LN:YCG1_YEAST] [AC:P25588:P25589:P27513:P87003] |
| | | | | | | | [GN:YCL061C:YCL61C/YCL60C] [OR:Saccharomyces cerevisiae] |
| | | | | | | | [SR:,Baker's yeast] [DE: 97.9 KDA PROTEIN IN CHA1-KRR1 |
| | | | | | | | INTERGENIC REGION] [SP:P25588:P25589:P27513:P87003] |
| | | | | | | | [DB:swissprot]>pir:[LN:S74279] |
| | | | | | | | [AC:S74279:S19392:S19391:S29373:S21360] [PN: protein |
| | | | | | | | YCL061c: protein YCL060c] [OR:Saccharomyces cerevisiae] |
| | | | | | | | [DB:pir2] [MP:3L] |
| smorf068 | 15 | 688 | 318 | 105 | 491 | 1.4E−46 | pir:[LN:S78709] [AC:S78709] [PN:protein YCL057c-a] |
| | | | | | | | [OR:Saccharomyces cerevisiae] [DB:pir2] [MP:3L]> |
| | | | | | | | gp:[GI:14588901] [LN:SCCHRIII] |
| | | | | | | | [AC:X59720:S43845:S49180:S58084:S93798] [PN: protein] |
| | | | | | | | [OR:Saccharomyces cerevisiae] [SR:baker's yeast] [DB:genpept- |
| | | | | | | | pln4] [DE:S.cerevisiae chromosome III complete DNA sequence.] |
| | | | | | | | [NT:ORF YCL057 - ORF - identified by SAGE] [LE:24032] |
| | | | | | | | [RE:24325] [DI:complement] |
| smorf070 | 16 | 689 | 393 | 130 | 582 | 3.1E−56 | gp:[GI:14588906] [LN:SCCHRIII] |
| | | | | | | | [AC:X59720:S43845:S49180:S58084:S93798] [PN: protein] |
| | | | | | | | [OR:Saccharomyces cerevisiae] [SR:baker's yeast] [DB:genpept- |
| | | | | | | | pin4] [DE:S.cerevisiae chromosome III complete DNA sequence.] |
| | | | | | | | [NT:ORF YCL034w -similarity to S.pombe] [LE:61658] [RE:62722] |
| | | | | | | | [DI:direct] |
| smorf079 | 17 | 690 | 180 | 59 | 188 | 1.2E−13 | gp:[GI:897808] [LN:SCPEL1GN] [AC:Z48162] |
| | | | | | | | [PN:phosphatidylserine synthase] [GN:PEL1] [OR:Saccharomyces |
| | | | | | | | cerevisiae] [SR:baker's yeast] [DB:genpept-pln4] |
| | | | | | | | [DE:Saccharomyces cerevisiae PEL1 gene.] [SP:P25578] [LE:414] |
| | | | | | | | [RE:1883] [DI:direct] |
| smorf080 | 18 | 691 | 636 | 211 | 649 | 2.5E−63 | sp:[LN:YCA2_YEAST] [AC:P25565] [GN:YCL002C:YCL2C] |
| | | | | | | | [OR:Saccharomyces cerevisiae] [SR:Baker's yeast] [DE: 14.4 KDA |
| | | | | | | | PROTEIN IN RER1-PEL1 INTERGENIC REGION] [SP:P25565] |
| | | | | | | | [DB:swissprot]>pir:[LN:S19357] [AC:S19357] [PN: membrane |
| | | | | | | | protein YCL002c] [GN:YCL002c] [CL:Saccharomyces membrane |
| | | | | | | | protein YCL002c] [OR:Saccharomyces cerevisiae] [DB:pir2] [MP:3L] |
| smorf082 | 19 | 692 | 375 | 124 | 423 | 2.2E−39 | gp:[GI:14588925] [LN:SCCHRIII] |
| | | | | | | | [AC:X59720:S43845:S49180:S58084:S93798] [PN:protein] |
| | | | | | | | [OR:Saccharomyces cerevisiae] [SR:baker's yeast] [DB:genpept- |
| | | | | | | | pln4] [DE:S.cerevisiae chromosome III complete DNA sequence.] |
| | | | | | | | [NT:ORF YCL001] [LE:113764] [RE:114018] [DI:direct] |
| smorf093 | 20 | 693 | 231 | 76 | 59 | 0.0038 | pir:[LN:T32594] [AC:T32594] [PN: protein C02B10.5] |
| | | | | | | | [GN:C02B10.5] [OR:Caenorhabditis elegans] [DB:pir2] [MP:4]> |
| | | | | | | | gp:[GI:2702380] [LN:AF038605] [AC:AF038605] [PN:protein |
| | | | | | | | C02B10.5] [GN:C02B10.5] [OR:Caenorhabditis elegans] |
| | | | | | | | [DB:genpept-inv2] [DE:Caenorhabditis elegans cosmid C02B10, |
| | | | | | | | complete sequence.] [NT:contains similarity to proteins with proline- |
| | | | | | | | rich] [LE:12715:13378:13555:13870] [RE:12897:13499:13813:14351] |
| | | | | | | | [DI:directJoin]>gp:[GI:2702380] [LN:AF038605] [AC:AF038605] [PN: |
| | | | | | | | protein C02B10.5] [GN:C02B10.5] [OR:Caenorhabditis elegans] |
| | | | | | | | [DB:genpept] [DE:Caenorhabditis elegans cosmid C02B10, complete |
| | | | | | | | sequence.] [NT:contains similarity to proteins with proline-rich] |
| | | | | | | | [LE:12715:13378:13555:13870] [RE:12897:13499:13813:14351] |
| | | | | | | | [DI:directJoin] |
| smorf098 | 21 | 694 | 210 | 69 | | | |
| smorf100 | 22 | 695 | 249 | 82 | | | |
| smorf101 | 23 | 696 | 165 | 54 | | | |
| smorf102 | 24 | 697 | 303 | 100 | 447 | 6.3E−42 | sp:[LN:STF1_YEAST] [AC:P01098] [GN:STF1:AIS2:YDL130BW] |
| | | | | | | | [OR:Saccharomyces cerevisiae] [SR:Baker's yeast] [DE:ATPASE |
| | | | | | | | STABILIZING FACTOR 9 KDA, MITOCHONDRIAL PRECURSOR] |
| | | | | | | | [SP:P01098] [DB:swissprot]>pir:[LN:IWBY9] |
| | | | | | | | [AC:JX0048:A01338:S25428] |
| smorf103 | 25 | 698 | 273 | 90 | 334 | 5.9E−30 | pir:[LN:S78710] [AC:S78710] [PN:protein YDL085c-a] |
| | | | | | | | [OR:Saccharomyces cerevisiae] [DB:pir2] [MP:4L] |
| smorf104 | 26 | 699 | 258 | 85 | | | |
| smorf108 | 27 | 700 | 324 | 107 | 479 | 2.6E−45 | gp:[GI:496672] [LN:SCDNCH2] [AC:X79489] [PN:D-104 protein] |
| | | | | | | | [GN:YBL0822a] [OR:Saccharomyces cerevisiae] [SR:Baker's yeast] |
| | | | | | | | [DB:genpept-pln4] [DE:S.cerevisiae genomic DNA, chromosome II |
| | | | | | | | from Y element to ILS1 gene.] [LE:27160] [RE:27474] |
| | | | | | | | [DI:complement] |
| smorf109 | 28 | 701 | 231 | 76 | 162 | 1E−11 | gp:[GI:12231165] [LN:SPBC32F12] [AC:AL023796] [PN: protein] |
| | | | | | | | [GN:SPBC32F12.15] [OR:Schizosaccharomyces pombe] [SR:fission |
| | | | | | | | yeast] [DB:genpept-pln4] [DE:S.pombe chromosome II cosmid |
| | | | | | | | c32F12.] [LE:24713] [RE:24919] [DI:direct] |
| smorf112 | 29 | 702 | 213 | 70 | | | |
| smorf118 | 30 | 703 | 231 | 76 | | | |
| smorf121 | 31 | 704 | 255 | 84 | 167 | 1.8E−11 | sp:[LN:YMS4_YEAST] [AC:Q05131] [GN:YMR034C:YM9973.08C] |
| | | | | | | | [OR:Saccharomyces cerevisiae] [SR:Baker's yeast] [DE: 48.4 KDA |
| | | | | | | | PROTEIN IN ARP9-IMP2 INTERGENIC REGION] [SP:Q05131] |
| | | | | | | | [DB:swissprot]>pir:[LN:S53951] [AC:S53951] [PN: membrane |
| | | | | | | | protein YMR034c: protein YM9973.08c] [GN:YMR034c] |
| | | | | | | | [OR:Saccharomyces cerevisiae] [DB:pir2] [MP:13R]>gp:[GI:798960] |
| | | | | | | | [LN:SC9973] [AC:Z49213:Z71257] [PN:] [OR:Saccharomyces |
| | | | | | | | cerevisiae] [SR:baker's yeast] [DB:genpept-pln4] [DE:S.cerevisiae |
| | | | | | | | chromosome XIII cosmid 9973.] [NT:YM9973.08c, len:434, CAI: |
| | | | | | | | 0.13] [SP:Q05131] [LE:11824] [RE:13128] [DI:complement] |
| smorf122 | 32 | 705 | 276 | 91 | 80 | 0.027 | sp:[LN:YD01_CLOAB] [AC:P33659] [GN:CAC1301] [OR:Clostridium |
| | | | | | | | acetobutylicum] [DE: protein CAC1301] [SP:P33659] [DB:swissprot]> |
| | | | | | | | gp:[GI:15024231] [LN:AE007642] [AC:AE007642:AE001437] |
| | | | | | | | [PN:membrane protein] [GN:CAC1301] [OR:Clostridium |
| | | | | | | | acetobutylicum] [DB:genpept-bct1] [DE:Clostridium acetobutylicum |
| | | | | | | | ATCC824 section 130 of 356 of the complete genome.] [LE:4514] |
| | | | | | | | [RE:5404] [DI:direct] |
| smorf123 | 33 | 706 | 171 | 56 | | | |
| smorf127 | 34 | 707 | 204 | 67 | | | |
| smorf137 | 35 | 708 | 294 | 97 | 484 | 7.6E−46 | pir:[LN:S78713] [AC:S78713] [PN:protein YDR322c-a] [GN:TIM11] |
| | | | | | | | [OR:Saccharomyces cerevisiae] [DB:pir2] [MP:4R] |
| smorf139 | 36 | 709 | 276 | 91 | 171 | 1.1E−12 | pir:[LN:T50242] [AC:T50242] [PN: Protein SPAC664.12c [imported]] |
| | | | | | | | [GN:SPAC664.12c] [OR:Schizosaccharomyces pombe] [DB:pir2] |
| | | | | | | | [MP:1]>gp:[GI:6692019] [LN:SPAC664] [AC:AL136235] [PN: |
| | | | | | | | protein] [GN:SPAC664.12c] [OR:Schizosaccharomyces pombe] |
| | | | | | | | [SR:fission yeast] [DB:genpept-pln4] [DE:S.pombe chromosome I |
| | | | | | | | cosmid c664.] [NT:SPAC664.12c, len:79] [LE:26362:26610] |
| | | | | | | | [RE:26523:26687] [DI:complement Join] |
| smorf140 | 37 | 710 | 396 | 131 | 668 | 2.4E−65 | sp:[LN:YRA1_YEAST] [AC:Q12159] |
| | | | | | | | [GN:YRA1:YDR381W:D9481.2:D9509.1] [OR:Saccharomyces |
| | | | | | | | cerevisiae] [SR:Baker's yeast] [DE:RNA ANNEALING PROTEIN |
| | | | | | | | YRA1] [SP:Q12159] [DB:swissprot]>gp:[GI:1912464] |
| | | | | | | | [LN:SCU72633] [AC:U72633] [PN:RNA annealing protein Yra1p] |
| | | | | | | | [GN:yra1] [OR:Saccharomyces cerevisiae] [SR:Baker's yeast] |
| | | | | | | | [DB:genpept-pln4] [DE:Saccharomyces cerevisiae RNA annealing |
| | | | | | | | protein Yra1p (yra1) gene, complete cds.] [LE:16:1067] |
| | | | | | | | [RE:300:1462] [DI:direct Join] |
| smorf144 | 38 | 711 | 270 | 89 | 81 | 0.0038 | pir:[LN:T28394] [AC:T28394] [PN: protein MSV234 [imported]] |
| | | | | | | | [OR:Melanoplus sanguinipes entomopoxvirus] [DB:pir2]> |
| | | | | | | | gp:[GI:4049784] [LN:AF063866] [AC:AF063866] [PN:ORF MSV234 |
| | | | | | | | hypthetical protein] [GN:MSV234] [OR:Melanoplus sanguinipes |
| | | | | | | | entomopoxvirus] [DB:genpept-vrl1] [DE:Melanoplus sanguinipes |
| | | | | | | | entomopoxvirus, complete genome.] [LE:201477] [RE:201830] |
| | | | | | | | [DI:complement] |
| smorf151 | 39 | 712 | 249 | 82 | 425 | 1.4E−39 | sp:[LN:YD5B_YEAST] [AC:P56508] [GN:YDR525BW] |
| | | | | | | | [OR:Saccharomyces cerevisiae] [SR:Baker's yeast] [DE: 9.2 kD |
| | | | | | | | PROTEIN IN SPS1-QCR7 INTERGENIC REGION] [SP:P56508] |
| | | | | | | | [DB:swissprot]>pir:[LN:S78716] [AC:S78716] [PN:protein YDR525w- |
| | | | | | | | a] [OR:Saccharomyces cerevisiae] [DB:pir2] [MP:4R] |
| smorf154 | 40 | 713 | 288 | 95 | | | |
| smorf167 | 41 | 714 | 306 | 101 | | | |
| smorf171 | 42 | 715 | 378 | 125 | | | |
| smorf172 | 43 | 716 | 279 | 92 | 454 | 1.1E−42 | pir:[LN:S78717] [AC:S78717] [PN:protein YEL020w-a] |
| | | | | | | | [OR:Saccharomyces cerevisiae] [DB:pir2] [MP:5L]>gp:[GI:3747026] |
| | | | | | | | [LN:AF093244] [AC:AF093244] [PN:import protein Tim9p] [GP:TIM9] |
| | | | | | | | [OR:Saccharomyces cerevisiae] [SR:baker's yeast] [DB:genpept- |
| | | | | | | | pln1] [DE:Saccharomyces cerevisiae import protein Tim9p (TIM9) |
| | | | | | | | gene, nucleargene encoding mitochondrial protein, complete cds.] |
| | | | | | | | [NT:mitochondrial intermembrane space protein] [LE:1] [RE:264] |
| | | | | | | | [DI :direct] |
| smorf181 | 44 | 717 | 360 | 119 | 488 | 2.9E−46 | pir:[LN:S78718] [AC:S78718] [PN:protein YER048w-a] |
| | | | | | | | [OR:Saccharomyces cerevisiae] [DB:pir2] [MP:5L] |
| smorf189 | 45 | 718 | 309 | 102 | | | |
| smorf201 | 46 | 719 | 243 | 80 | 82 | 0.021 | gp:[GI:3264834] [LN:AF072541] [AC:AF072541] [PN:xylitol |
| | | | | | | | dehydrogenase] [GN:xdh] [FN:xylose utilisation] [OR:Candida sp. |
| | | | | | | | HA167] [DB:genpept-pln1] [EC:1.1.1.9] [DE:Galactocandida |
| | | | | | | | mastotermitis xylitol dehydrogenase (xdh) gene, complete cds.] [NT:a |
| | | | | | | | member of the medium chain dehydrogenase] [LE:301:373] |
| | | | | | | | [RE:312:1422] [DI:directJoin] |
| smorf207 | 47 | 720 | 222 | 73 | 316 | 4.8E−28 | pir:[LN:S71066] [AC:S71066:S11265] [PN:ribosomal protein L29.e, |
| | | | | | | | cytosolic:protein YFR032c-a:robosomal protein YL43] [CL:rat |
| | | | | | | | ribosomal protein L29] [OR:Saccharomyces cerevisiae] [DB:pir2] |
| | | | | | | | [MP:6R] |
| smorf217 | 48 | 721 | 303 | 100 | 377 | 1.6E−34 | sp:[LN:YGW1_YEAST] [AC:P53088:Q92322] [GN:YGL211W] |
| | | | | | | | [OR:Saccharomyces cerevisiae] [SR:Baker's yeast] [DE: 35.5 KDA |
| | | | | | | | PROTEIN IN VAM7-YPT32 INTERGENIC REGION] |
| | | | | | | | [SP:P53088:Q92322] [DB:swissprot]>pir:[LN:S64230] |
| | | | | | | | [AC:S71668:S71671:S64230] [PN: protein YGL211w: protein |
| | | | | | | | G1125] [CL:conserved protein MJ1157] [OR:Saccharomyces |
| | | | | | | | cerevisiae] [DB:pir2] [MP:7L]>gp:[GI:1655726] [LN:SCU33754] |
| | | | | | | | [AC:U33754] [PN:] [OR:Saccharomyces cerevisiae] [SR:baker's |
| | | | | | | | yeast strain=S288C-27] [DB:genpept-pln4] [DE:Saccharomyces |
| | | | | | | | cerevisiae Vam7p (VAM7), ras-like GTPase (YPT11) and MIG1-like |
| | | | | | | | zinc finger protein (MLZ1) genes, complete cds and Sip2p(SPM2) |
| | | | | | | | gene, partial cds.] [NT:orf-1] [LE:2003] [RE:2956] [DI:direct] |
| smorf226 | 49 | 722 | 195 | 64 | 77 | 0.034 | pir:[LN:D82461] [AC:D82461] [PN: protein VCA0413 [imported]] |
| | | | | | | | [GN:VCA0413] [OR:Vibrio cholerae] [DB:pir2] [MP:2]> |
| | | | | | | | gp:[GI:9657815] [LN:AE004376] [AC:AE004376:AE003853] [PN: |
| | | | | | | | protein] [GN:VCA0413] [OR:Vibrio cholerae] [DB:genpept-bct1] |
| | | | | | | | [DE:Vibrio cholerae chromosome II, section 33 of 93 of the |
| | | | | | | | complete chromosome.] [NT:identified by Glimmer2;] [LE:1146] |
| | | | | | | | [RE:1799] [DI:direct] |
| smorf247 | 50 | 723 | 219 | 72 | | | |
| smorf250 | 51 | 724 | 228 | 75 | 80 | 0.0049 | pir:[LN:F81931] [AC:F81931] [PN: protein NMA0858 [imported]] |
| | | | | | | | [GN:NMA0858] [CL:Neisseria meningitidis protein NMB0650] |
| | | | | | | | [OR:Neisseria meningitidis] [DB:pir2]>gp:[GI:7379574] |
| | | | | | | | [LN:NMA3Z2491] [AC:AL162754:AL157959] [PN: protein NMA0858] |
| | | | | | | | [GN:NMA0858] [OR:Neisseria meningitidis Z2491] [DB:genpept- |
| | | | | | | | bct3] [DE:Neisseria meningitidis serogroup A strain Z2491 complete |
| | | | | | | | genome; segment 3/7.] [NT:NMA0858, len: 129 aa; similar to |
| | | | | | | | NMA0856] [LE:145998] [RE:146387] [DI:direct] |
| smorf268 | 52 | 725 | 195 | 64 | 304 | 9E−27 | pir:[LN:S78745] [AC:S78745] [PN:protein YHR072w-a] |
| | | | | | | | [OR:Saccharomyces cerevisiae] [DB:pir2] [MP:8R] |
| smorf274 | 53 | 726 | 231 | 76 | 75 | 0.036 | pir:[LN:S65828] [AC:S65828] [PN: movement protein] [CL:potato |
| | | | | | | | leaf roll virus genomE−linked protein] [OR:beet mild yellowing virus] |
| | | | | | | | [DB:pir2]>gp:[GI:951034] [LN:MYVRNA] [AC:X83110] [PN: protein |
| | | | | | | | P5] [OR:Beet western yellows virus] [DB:genpept-vrl2] [DE:Beet mild |
| | | | | | | | yellowing virus genomic RNA.] [LE:3628] [RE:4155] [DI:direct] |
| smorf279 | 54 | 727 | 384 | 127 | 473 | 1.1E−44 | gp:[GI:6760480] [LN:YSCH9315] [AC:U10398:U00093] [PN:Yhr132w- |
| | | | | | | | ap] [GN:YHR132W-A] [OR:Saccharomyces cerevisiae] [SR:baker's |
| | | | | | | | yeast] [DB:genpept-pln4] [DE:Saccharomyces cerevisiae |
| | | | | | | | chromosome VIII cosmid 9315.] [NT:YHR132W-A:Added Jan 2000 |
| | | | | | | | from work of A. Horiuchi] [LE:16851] [RE:17246] [DI:direct] |
| smorf283 | 55 | 728 | 240 | 79 | 81 | 0.027 | pir:[LN:A70144] [AC:A70144] [PN:protein BB0354] [OR:Borrelia |
| | | | | | | | burgdorferi] [SR:,Lyme disease spirochete] [DB:pir2]> |
| | | | | | | | gp:[GI:2688259] [LN:AE001141] [AC:AE001141:AE000783] [PN:B. |
| | | | | | | | burgdorferi coding region BB0354] [GN:BB0354] [OR:Borrelia |
| | | | | | | | burgdorferi] [SR:Lyme disease spirochete] [DB:genpept-bct1] |
| | | | | | | | [DE:Borrelia burgdorferi (section 27 of 70) of the complete genome.] |
| | | | | | | | [NT: protein; identified by Glimmer;] [LE:8770] [RE:9810] |
| | | | | | | | [DI:complement] |
| smorf286 | 56 | 729 | 144 | 47 | | | |
| smorf288 | 57 | 730 | 192 | 63 | | | |
| smorf294 | 58 | 731 | 345 | 114 | 431 | 3.1E−40 | sp:[LN:H150_YEAST] [AC:P32478:Q03179] [GN:HSP150:PIR2] |
| | | | | | | | [OR:Saccharomyces cerevisiae] [SR:,Baker's yeast] [DE:150 KDA |
| | | | | | | | HEAT SHOCK GLYCOPROTEIN PRECURSOR] |
| | | | | | | | [SP:P32478:Q03179] [DB:swissprot] |
| smorf298 | 59 | 732 | 201 | 66 | | | |
| smorf301 | 60 | 733 | 312 | 103 | | | |
| smorf303 | 61 | 734 | 360 | 119 | 220 | 7.2E−18 | sp:[LN:YEQ2_YEAST] [AC:P40046] [GN:YER072W] |
| | | | | | | | [OR:Saccharomyces cerevisiae] [SR:Baker's yeast] [DE: 14.4 KDA |
| | | | | | | | PROTEIN IN RNR1-ALD3 INTERGENIC REGION] [SP:P40046] |
| | | | | | | | [DB:swissprot]>pir:[LN:S50575] [AC:S50575] [PN: protein |
| | | | | | | | YER072w] [GN:YER072w] [OR:Saccharomyces cerevisiae] [DB:pir2] |
| | | | | | | | [MP:5R]>gp:[GI:603308] [LN:SCE6592] [AC:U18813:U00092] |
| | | | | | | | [PN:Yer072wp] [GN:YER072W] [OR:Saccharomyces cerevisiae] |
| | | | | | | | [SR:baker's yeast] [DB:genpept-pln4] [DE:Saccharomyces |
| | | | | | | | cerevisiae chromosome V lambda clones 6592, 4678, 4742, and |
| | | | | | | | 3612.] [LE:42146] [RE:42535] [DI:direct] |
| smorf313 | 62 | 735 | 336 | 111 | 103 | 0.000018 | pir:[LN:T37538] [AC:T37538] [PN: protein SPAC11E3.10] |
| | | | | | | | [GN:SPAC11E3.10] [OR:Schizosaccharomyces pombe] [DB:pir2] |
| | | | | | | | [MP:1]>gp:[GI:4539235] [LN:SPAC11E3] [AC:Z98595] [PN: protein] |
| | | | | | | | [GN:SPAC11E3.10] [OR:Schizosaccharomyces pombe] [SR:fission |
| | | | | | | | yeast] [DB:genpept-pln4] [DE:S.pombe chromosome I cosmid |
| | | | | | | | c11E3.] [NT:SPAC11E3.10, len:162] [SP:O13689] |
| | | | | | | | [LE:23704:23847:24038:24272] [RE:23765:23870:24224:24301] |
| | | | | | | | [DI:directJoin] |
| smorf315 | 63 | 736 | 294 | 97 | 441 | 2.7E−41 | pir:[LN:S78075] [AC:S78075] [PN:protein YJR135w-a] |
| | | | | | | | [GN:YJR135w-a] [CL: protein SPAC13G6.04] [OR:Saccharomyces |
| | | | | | | | cerevisiae] [DB:pir2] [MP:10R] |
| smorf318 | 64 | 737 | 174 | 57 | | | |
| smorf323 | 65 | 738 | 288 | 95 | 288 | 3.3E−24 | gp:[GI:2980815] [LN:SCYKL200C] [AC:Z28200:Y13137] [GN:MNN4] |
| | | | | | | | [OR:Saccharomyces cerevisiae] [SR:baker's yeast] [DB:genpept- |
| | | | | | | | pln4] [DE:S.cerevisiae chromosome XI reading frame ORF |
| | | | | | | | YKL200c.] [NT:ORF YKL201c] [LE:<1] [RE:1917] [DI:complement] |
| smorf324 | 66 | 739 | 216 | 71 | 81 | 0.024 | pir:[LN:T30138] [AC:T30138] [PN: protein E02C12.2] |
| | | | | | | | [GN:E02C12.2] [CL:Caenorhabditis elegans protein K07C6.10] |
| | | | | | | | [OR:Caenorhabditis elegans] [DB:pir2]>gp:[GI:1123057] |
| | | | | | | | [LN:U41995] [AC:U41995] [PN: protein E02C12.2] [GN:E02C12.2] |
| | | | | | | | [OR:Caenorhabditis elegans] [DB:genpept-inv4] [DE:Caenorhabditis |
| | | | | | | | elegans cosmid E02C12, complete sequence.] |
| | | | | | | | [LE:4721:4830:5037:5223] [RE:4762:4990:5180:5529] [DI:directJoin] |
| smorf327 | 67 | 740 | 273 | 90 | 465 | 7.8E−44 | pir:[LN:S78725] [AC:S78725:S78074] [PN:protein YKL053c-a] |
| | | | | | | | [OR:Saccharomyces cerevisiae] [SR:strain S288C, strain S288C] |
| | | | | | | | [SR:strain S288C,] [DB:pir2] [MP:11L]>gp:[GI:2980812] |
| | | | | | | | [LN:SCYKL053W] [AC:Z28052:Y13137] [OR:Saccharomyces |
| | | | | | | | cerevisiae] [SR:baker's yeast] [DB:genpept-pln4] [DE:S.cerevisiae |
| | | | | | | | chromosome XI reading frame ORF YKL053w.] [NT:ORF YKL053c- |
| | | | | | | | a] [LE:429] [RE:689] [DI:complement]>gp:[GI:2980813] |
| | | | | | | | [LN:SCYKL054C] [AC:Z28054:Y13137] [OR:Saccharomyces |
| | | | | | | | cerevisiae] [SR:baker's yeast] [DB:genpept-pln4] [DE:S.cerevisiae |
| | | | | | | | chromosome XI reading frame ORF YKL054c.] [NT:ORF YKL053c-a] |
| | | | | | | | [LE:3025] [RE:3285] [DI:complement] |
| smorf337 | 68 | 741 | 273 | 90 | 73 | 0.043 | pir:[LN:H71248] [AC:H71248] [PN: protein PH0247] [GN:PH0247] |
| | | | | | | | [OR:Pyrococcus horikoshii] [DB:pir2]>gp:[GI:3256636] |
| | | | | | | | [LN:AP000001] |
| | | | | | | | [AC:AP000001:AB009465:AB009464:AB009466:AB009467:AB0094 |
| | | | | | | | 68:AB009469] [PN:153 aa long protein] [GN:PH0247] |
| | | | | | | | [OR:Pyrococcus horikoshii] [SR:Pyrococcus horikoshii (strain:OT3) |
| | | | | | | | DNA] [DB:genpept-bct2] [DE:Pyrococcus horikoshii OT3 genomic |
| | | | | | | | DNA, 1-287000 nt. position (1/7).] [LE:222381] [RE:222842] |
| | | | | | | | [DI:complement] |
| smorf350 | 69 | 742 | 309 | 102 | 543 | 4.2E−52 | pir:[LN:S78727] [AC:S78727] [PN:protein YLL018c-a] |
| | | | | | | | [OR:Saccharomyces cerevisiae] [DB:pir2] [MP:12L] |
| smorf352 | 70 | 743 | 192 | 63 | | | |
| smorf363 | 71 | 744 | 228 | 75 | | | |
| smorf382 | 72 | 745 | 219 | 72 | | | |
| smorf392 | 73 | 746 | 390 | 129 | | | |
| smorf398 | 74 | 747 | 192 | 63 | | | |
| smorf421 | 75 | 748 | 150 | 49 | | | |
| smorf439 | 76 | 749 | 276 | 91 | 220 | 7.2E−18 | gp:[GI:13359451] [LN:AB049723] [AC:AB049723] [PN:senescence- |
| | | | | | | | associated protein] [GN:ssa-13] [OR:Pisum sativum] [SR:Pisum |
| | | | | | | | sativum (cultivar:Ichihara wase) immatured pods pods cDNA t] |
| | | | | | | | [DB:genpept-pln1] [DE:Pisum sativum ssa-13 mRNA for senescence- |
| | | | | | | | associated protein, partial cds.] [LE:<117] [RE:965] [DI:direct] |
| smorf483 | 77 | 750 | 279 | 92 | 175 | 5.5E−13 | gp:[GI:13359451] [LN:AB049723] [AC:AB049723] [PN: senescence- |
| | | | | | | | associated protein] [GN:ssa-13] [OR:Pisum sativum] [SR:Pisum |
| | | | | | | | sativum (cultivar:Ichihara wase) immatured pods pods cDNA t] |
| | | | | | | | [DB:genpept-pln1] [DE:Pisum sativum ssa-13 mRNA for |
| | | | | | | | senescence-associated protein, partial cds.] [LE:<117] [RE:965] |
| | | | | | | | [DI:direct] |
| smorf494 | 78 | 751 | 156 | 51 | | | |
| smorf499 | 79 | 752 | 240 | 79 | | | |
| smorf505 | 80 | 753 | 264 | 87 | 70 | 0.033 | gp:[GI:2708565] [LN:AF033594] [AC:AF033594] [PN:maturase] |
| | | | | | | | [GN:matK] [OR:Chloroplast Paeonia anomala] [SR:Paeonia |
| | | | | | | | anomala] [DB:genpept-pln1] [DE:Paeonia anomala maturase (matK) |
| | | | | | | | gene, chloroplast gene encoding chloroplast protein, complete cds.] |
| | | | | | | | [LE:1] [RE:1491] [DI:direct] |
| smorf508 | 81 | 754 | 750 | 249 | 1248 | 8.3E−127 | sp:[LN:RM15_YEAST] [AC:P36523:P89101:O13551] |
| | | | | | | | [GN:MRPL15:YLR312BW] [OR:Saccharomyces cerevisiae] |
| | | | | | | | [SR:,Baker's yeast] [DE:60S RIBOSOMAL PROTEIN L15, |
| | | | | | | | MITOCHONDRIAL PRECURSOR (YML15) (MRP-L15)] |
| | | | | | | | [SP:P36523:P89101:O13551] [DB:swissprot]>pir:[LN:S72159] |
| | | | | | | | [AC:S72159:S17264:S78017] [PN:ribosomal protein YmL15 |
| | | | | | | | precursor, mitochondrial:protein YLR312w-a] [GN:MRPL15] |
| | | | | | | | [OR:Saccharomyces cerevisiae] [DB:pir2] [MP:12R]> |
| | | | | | | | gp:[GI:2258171] [LN:YSCL8543] [AC:U20618:Y13138] |
| | | | | | | | [PN:Mrpl15p: mitochondrial ribosomal protein YmL15] [GN:MRPL15] |
| | | | | | | | [OR:Saccharomyces cerevisiae] [SR:baker's yeast strain=S288C |
| | | | | | | | (AB972)] [DB:genpept-pln4] [DE:Saccharomyces cerevisiae |
| | | | | | | | chromosome XII cosmid 8543.] [NT:Ylr312w-ap] [LE:4494] |
| | | | | | | | [RE:5255] [DI:direct] |
| smorf509 | 82 | 755 | 435 | 144 | 599 | 4.9E−58 | gp:[GI:2258412] [LN:AF008236] [AC:AF008236] [PN:Sph1p] |
| | | | | | | | [GN:SPH1] [OR:Saccharomyces cerevisiae] [SR:baker's yeast] |
| | | | | | | | [DB:genpept-pln1] [DE:Saccharomyces cerevisiae Sph1p (SPH1) |
| | | | | | | | gene, complete cds.] [NT:has 3 regions similar to S. cerevisiae |
| | | | | | | | Spa2p;] [LE:1] [RE:1947] [DI:direct] |
| smorf511 | 83 | 756 | 231 | 76 | | | |
| smorf514 | 84 | 757 | 288 | 95 | 83 | 0.016 | gp:[GI:7293848] [LN:AE003519] [AC:AE003519:AE002602] |
| | | | | | | | [GN:CG6843] [OR:Drosophila melanogaster] [SR:fruit fly] |
| | | | | | | | [DB:genpept-inv2] [DE:Drosophila melanogaster genomic scaffold |
| | | | | | | | 142000013386050 section 49 of 54, complete sequence.] |
| | | | | | | | [NT:CG6843 gene product] [LE:258810] [RE:259832] [DI:direct] |
| smorf519 | 85 | 758 | 318 | 105 | 89 | 0.0063 | pir:[LN:E71620] [AC:E71620] [PN: protein PFB0225c] |
| | | | | | | | [GN:PFB0225c] [OR:Plasmodium falciparum] [DB:pir2]> |
| | | | | | | | gp:[GI:3845128] [LN:AE001381] [AC:AE001381:AE001362] [PN: |
| | | | | | | | protein] [GN:PFB0225c] [OR:Plasmodium falciparum] [SR:malaria |
| | | | | | | | parasite P. falciparum] [DB:genpept-inv1] [DE:Plasmodium |
| | | | | | | | falciparum chromosome 2, section 18 of 73 of the complete |
| | | | | | | | sequence.] [NT:predicted by GlimmerM] [LE:7198] [RE:8724] |
| | | | | | | | [DI:complement] |
| smorf523 | 86 | 759 | 195 | 64 | 314 | 7.8E−28 | sp:[LN:AT18_YEAST] [AC:P81450] [GN:ATP18:YML081BC] |
| | | | | | | | [OR:Saccharomyces cerevisiae] [SR:,Baker's yeast] [EC:3.6.1.34] |
| | | | | | | | [DE:I SUBUNIT)] [SP:P81450] [DB:swissprot]>pir:[LN:S78730] |
| | | | | | | | [AC:S78730] [PN:protein YML081c-a] [OR:Saccharomyces |
| | | | | | | | cerevisiae] [DB:pir2] [MP:13L]>gp:[GI:3329486] [LN:AF073791] |
| | | | | | | | [AC:AF073791] [PN:ATP synthase subunit i] [GN:ATP18] |
| | | | | | | | [OR:Saccharomyces cerevisiae] [SR:baker's yeast] [DB:genpept- |
| | | | | | | | pln1] [DE:Saccharomyces cerevisiae ATP Synthase subunit i |
| | | | | | | | (ATP18) gene, nuclear gene encoding mitochondrial protein, |
| | | | | | | | complete cds.] [NT:Atp 18p] [LE:16] [RE:195] [DI:direct] |
| smorf526 | 87 | 760 | 201 | 66 | | | |
| smorf530 | 88 | 761 | 327 | 108 | 488 | 2.9E−46 | pir:[LN:S53949] [AC:S53949] [PN: protein YM9973.06] |
| | | | | | | | [OR:Saccharomyces cerevisiae] [DB:pir4] [MP:13R]>gp:[GI:798958] |
| | | | | | | | [LN:SC9973] [AC:Z49213:Z71257] [PN:] [OR:Saccharomyces |
| | | | | | | | cerevisiae] [SR:baker's yeast] [DB:genpept-pln4] [DE:S.cerevisiae |
| | | | | | | | chromosome XIII cosmid 9973.] [NT:YM9973.06, orf?len:96, CAI: |
| | | | | | | | 0.08] [LE:9719] [RE:10009] [DI:direct] |
| smorf532 | 89 | 762 | 273 | 90 | | | |
| smorf540 | 90 | 763 | 216 | 71 | 78 | 0.021 | pir:[LN:T44148] [AC:T44148] [PN: protein B4 [imported]] [OR:human |
| | | | | | | | herpesvirus 6] [SR:strain Z29, stain Z29] [SR:strain Z29,] [DB:pir2]> |
| | | | | | | | gp:[GI:5733517] [LN:AF157706] |
| | | | | | | | [AC:AF157706:L13162:L14772:L16947] [PN:B4] [GN:B4] |
| | | | | | | | [OR:Human herpesvirus 6B] [DB:genpept-vrl1] [DE:Human |
| | | | | | | | herpesvirus 6B strain Z29, complete genome.] [LE:8911] [RE:9492] |
| | | | | | | | [DI:complement] |
| smorf543 | 91 | 764 | 270 | 89 | 157 | 3.4E−11 | pir:[LN:T37930] [AC:T37930] [PN: lysine-rich protein] |
| | | | | | | | [GN:SPAC1952.02] [OR:Schizosaccharomyces pombe] [DB:pir2] |
| | | | | | | | [MP:1]>gp:[GI:5731935] [LN:SPAC1952] [AC:AL109820] [PN: |
| | | | | | | | lysine-rich protein] [GN:SPAC1952.02] [OR:Schizosaccharomyces |
| | | | | | | | pombe] [SR:fission yeast] [DB:genpept-pln4] [DE:S.pombe |
| | | | | | | | chromosome I cosmid c1952.] [NT:SPAC1952.02, len:224, highly |
| | | | | | | | charged C-term] [LE:1052:1313:1470] [RE:1231:1405:1871] |
| | | | | | | | [DI:directJoin] |
| smorf544 | 92 | 765 | 234 | 77 | | | |
| smorf556 | 93 | 766 | 222 | 73 | | | |
| smorf561 | 94 | 767 | 228 | 75 | | | |
| smorf564 | 95 | 768 | 486 | 161 | 760 | 4.3E−75 | sp:[LN:CMC1_YEAST] [AC:P48233] [GN:YNL083W:N2312] |
| | | | | | | | [OR:Saccharomyces cerevisiae] [SR:,Baker's yeast] [DE: calcium- |
| | | | | | | | binding mitochondrial carrier YNL083W] [SP:P48233] [DB:swissprot] |
| smorf570 | 96 | 769 | 336 | 111 | 224 | 2.7E−18 | gp:[GI:12833197] [LN:AK002884] [AC:AK002884] [OR:Mus |
| | | | | | | | musculus] [SR:Mus musculus (strain:C57BL/6J) adult male kidney |
| | | | | | | | cDNA to mRNA] [DB:genpept-htc] [DE:Mus musculus adult male |
| | | | | | | | kidney cDNA, RIKEN full-length enriched library, clone:0610041E09] |
| smorf572 | 97 | 770 | 270 | 89 | 369 | 1.2E−33 | pir:[LN:S78735] [AC:S78735] [PN:protein YNR032c-a] |
| | | | | | | | [OR:Saccharomyces cerevisiae] [DB:pir2] [MP:14R] |
| smorf577 | 98 | 771 | 216 | 71 | | | |
| smorf580 | 99 | 772 | 174 | 57 | 90 | 0.0054 | sp:[LN:YIQ6_YEAST] [AC:P40445] [GN:YIL166C] |
| | | | | | | | [OR:Saccharomyces cerevisiae] [SR:,Baker's yeast] [DE: |
| | | | | | | | TRANSPORTER YIL166C] [SP:P40445] [DB:swissprot]> |
| | | | | | | | pir:[LN:S50361] [AC:S50361] [PN: membrane protein YIL166c: |
| | | | | | | | protein YI9402.09c] [GN:YIL166c] [OR:Saccharomyces cerevisiae] |
| | | | | | | | [DB:pir2] [MP:9L]>gp:[GI:600811] [LN:SC9402] |
| | | | | | | | [AC:Z46921:Z47047] [PN:] [OR:Saccharomyces cerevisiae] |
| | | | | | | | [SR:baker's yeast] [DB:genpept-pln4] [DE:S.cerevisiae chromosome |
| | | | | | | | IX cosmid 9402 and left telomere.] [NT:YI9402.09c, orf, len:542, |
| | | | | | | | CAI:0.14] [SP:P40445] [LE:30938] [RE:32566] [DI:complement] |
| smorf587 | 100 | 773 | 222 | 73 | 356 | 2.8E−32 | sp:[LN:AT19_YEAST] [AC:P81451] [GN:ATP19:YOL078BW] |
| | | | | | | | [OR:Saccharomyces cerevisiae] [SR:,Baker's yeast] [EC:3.6.1.34] |
| | | | | | | | [DE:ATP SYNTHASE K CHAIN, MITOCHONDRIAL,] [SP:P81451] |
| | | | | | | | [DB:swissprot]>pir:[LN:S78739] [AC:S78739] [PN:protein YOL077w- |
| | | | | | | | a] [OR:Saccharomyces cerevisiae] [DB:pir2] [MP:15L] |
| smorf590 | 101 | 774 | 255 | 84 | | | |
| smorf591 | 102 | 775 | 330 | 109 | 78 | 0.0079 | sp:[LN:AT19_YEAST] [AC:P81451] [GN:ATP19:YOL078BW] |
| | | | | | | | [OR:Saccharomyces cerevisiae] [SR:,Baker's yeast] [EC:3.6.1.34] |
| | | | | | | | [DE:ATP SYNTHASE K CHAIN, MITOCHONDRIAL,] [SP:P81451] |
| | | | | | | | [DB:swissprot]>pir:[LN:S78739] [AC:S78739] [PN:protein YOL077w- |
| | | | | | | | a] [OR:Saccharomyces cerevisiae] [DB:pir2] [MP:15L] |
| smorf598 | 103 | 776 | 279 | 92 | | | |
| smorf601 | 104 | 777 | 381 | 126 | | | |
| smorf605 | 105 | 778 | 213 | 70 | | | |
| smorf621 | 106 | 779 | 528 | 175 | 656 | 4.5E−64 | gp:[GI:3618355] [LN:AB017593] [AC:AB017593] [GN:MBF1] |
| | | | | | | | [OR:Saccharomyces cerevisiae] [SR:Saccharomyces cerevisiae |
| | | | | | | | (strain:KT130) DNA] [DB:genpept-pln1] [DE:Saccharomyces |
| | | | | | | | cerevisiae MBF1 gene, complete cds.] [LE:64] [RE:519] [DI:direct] |
| smorf625 | 107 | 780 | 237 | 78 | 73 | 0.027 | gp:[GI:12718480] [LN:NCB18D24] [AC:AL513466] [PN: protein] |
| | | | | | | | [GN:B18D24.110] [OR:Neurospora crassa] [DB:genpept-pln3] |
| | | | | | | | [DE:Neurospora crassa DNA linkage group V BAC contig B18D24.] |
| | | | | | | | [LE:93849] [RE:94196] [DI:direct] |
| smorf626 | 108 | 781 | 357 | 118 | | | |
| smorf631 | 109 | 782 | 282 | 93 | | | |
| smorf632 | 110 | 783 | 222 | 73 | | | |
| smorf640 | 111 | 784 | 345 | 114 | | | |
| smorf643 | 112 | 785 | 252 | 83 | | | |
| smorf644 | 113 | 786 | 402 | 133 | 487 | 3.6E−46 | sp:[LN:YP83_YEAST] [AC:O14464] [GN:YPL183BW] |
| | | | | | | | [OR:Saccharomyces cerevisiae] [SR:,Baker's yeast] [DE: 60S |
| | | | | | | | RIBOSOMAL PROTEIN YPL183BW, MITOCHONDRIAL |
| | | | | | | | PRECURSOR] [SP:O14464] [DB:swissprot]>pir:[LN:S72254] |
| | | | | | | | [AC:S72254] [PN:ribosomal protein L36, mitochondrial:protein |
| | | | | | | | YPL 183w-a] [CL:Escherichia coli ribosomal protein L36] |
| | | | | | | | [OR:Saccharomyces cerevisiae] [DB:pir2] [MP:16L]> |
| | | | | | | | gp:[GI:2326835] [LN:SCYPL183C] [AC:Z73539:U00094] |
| | | | | | | | [OR:Saccharomyces cerevisiae] [SR:baker's yeast] [DB:genpept- |
| | | | | | | | pln4] [DE:S.cerevisiae chromosome XVI reading frame ORF |
| | | | | | | | YPL 183c.] [NT:ORF YPL 183w-a] [SP:O14464] [LE:1307] [RE:1588] |
| | | | | | | | [DI:direct]>gp:[GI:2326836] [LN:SCYPL184C] [AC:Z73540:U00094] |
| | | | | | | | [OR:Saccharomyces cerevisiae] [SR:baker's yeast] [DB:genpept- |
| | | | | | | | pln4] [DE:S.cerevisiae chromosome XVI reading frame ORF |
| | | | | | | | YPL 184c.] [NT:ORF YPL 183w-a] [SP:O14464] [LE:3447] [RE:3728] |
| | | | | | | | [DI:direct] |
| smorf655 | 114 | 787 | 195 | 64 | | | |
| smorf660 | 115 | 788 | 261 | 86 | 346 | 3.2E−31 | pir:[LN:S78742] [AC:S78742] [PN:protein YCR018c-a:protein |
| | | | | | | | YCR019w] [OR:Saccharomyces cerevisiae] [DB:pir2] [MP:3R]> |
| | | | | | | | gp:[GI:14588933] [LN:SCCHRIII] |
| | | | | | | | [AC:X59720:S43845:S49180:S58084:S93798] [PN: protein] |
| | | | | | | | [OR:Saccharomyces cerevisiae] [SR:baker's yeast] [DB:genpept- |
| | | | | | | | pln4] [DE:S.cerevisiae chromosome III complete DNA sequence.] |
| | | | | | | | [NT:ORF YCR018c-a- ORF -identified by] [LE:151602] |
| | | | | | | | [RE:151856] [DI:complement] |
| smorf664 | 116 | 789 | 447 | 148 | 546 | 2E−52 | pir:[LN:S59764] [AC:S59764] [PN: membrane protein YPR098c: |
| | | | | | | | protein P8283. 13] [GN:YPR098c] [CL:Saccharomyces membrane |
| | | | | | | | protein YPR098c] [OR:Saccharomyces cerevisiae] [DB:pir2] |
| | | | | | | | [MP:16R]>gp:[GI:914970] [LN:YSCP8283] [AC:U32445:U00094] |
| | | | | | | | [PN:Ypr098cp] [GN:YPR098C] [OR: Saccharomyces cerevisiae] |
| | | | | | | | [SR:baker's yeast strain=S288C (AB972)] [DB:genpept-pln4] |
| | | | | | | | [DE:Saccharomyces cerevisiae chromosome XVI cosmid 8283.] |
| | | | | | | | [LE:509] [RE:835] [DI:complement] |
| smorf667 | 117 | 790 | 261 | 86 | | | |
| smorf669 | 118 | 791 | 159 | 52 | 267 | 7.5E−23 | sp:[LN:OM05_YEAST] [AC:P80967] [GN:TOM5] |
| | | | | | | | [OR:Saccharomyces cerevisiae] [SR:Baker's yeast] |
| | | | | | | | [DE:MITOCHONDRIAL IMPORT RECEPTOR SUBUNIT TOM5] |
| | | | | | | | [SP:P80967] [DB:swissprot]>pir:[LN:S77712] [AC:S77712] |
| | | | | | | | [PN:mitochondrial outer membrane protein TOM5:protein YPR133w- |
| | | | | | | | a] [GN:TOM5:YPR133w-a] [OR:Saccharomyces cerevisiae] |
| | | | | | | | [DB:pir2] [MP:16R] |
| smorf672 | 119 | 792 | 252 | 83 | | | |
| smorf001 | 120 | 793 | 258 | 85 | 106 | 0.0000086 | pir:[LN:S62023] [AC:S62023] [PN: membrane protein YDR544c: |
| | | | | | | | protein D3703.5] [GN:YDR544c] [OR:Saccharomyces cerevisiae] |
| | | | | | | | [DB:pir2] [MP:4R]>gp:[GI:1165299] [LN:SCU43834] |
| | | | | | | | [AC:U43834:Z71256] [PN:Ydr544cp] [GN:YDR544C] |
| | | | | | | | [OR:Saccharomyces cerevisiae] [SR:baker's yeast] [DB:genpept- |
| | | | | | | | pln4] [DE:Saccharomyces cerevisiae chromosome IV lambda 3073 |
| | | | | | | | and flankingregion extending into right telomere.] [NT:similar to 17.1 |
| | | | | | | | KD protein in PUR5] [LE:15357] [RE:15785] [DI:complement] |
| smorf002 | 121 | 794 | 228 | 75 | | | |
| smorf004 | 122 | 795 | 216 | 71 | 74 | 0.021 | gp:[GI:3511143] [LN:AF061244] [AC:AF061244] [PN:] |
| | | | | | | | [OR:Mitochondrion Agrocybe aegerita] [SR:Agrocybe aegerita] |
| | | | | | | | [DB:genpept-pln1] [DE:Agrocybe aegerita B type DNA polymerase |
| | | | | | | | (Mtpol) gene, complete cds; tRNA-Asn gene, complete sequence; |
| | | | | | | | and genes, mitochondrialgenes for mitochondrial products.] |
| | | | | | | | [NT:ORF C] [LE:7248] [RE:7571] [DI:direct] |
| smorf005 | 123 | 796 | 144 | 47 | | | |
| smorf006 | 124 | 797 | 126 | 41 | | | |
| smorf007 | 125 | 798 | 213 | 70 | | | |
| smorf008 | 126 | 799 | 96 | 31 | | | |
| smorf009 | 127 | 800 | 168 | 55 | | | |
| smorf011 | 128 | 801 | 216 | 71 | 62 | 0.022 | gp:[GI:13345829] [LN:AF332096] [AC:AF332096] [PN:twisted |
| | | | | | | | gastrulation protein] [GN:ztsg1] [OR:Danio rerio] [SR:zebrafish] |
| | | | | | | | [DB:genpept-vrt] [DE:Danio rerio twisted gastrulation protein (ztsg1) |
| | | | | | | | mRNA, completecds.] [NT:secreted protein] [LE:32] [RE:700] |
| | | | | | | | [DI:direct] |
| smorf012 | 129 | 802 | 255 | 84 | 111 | 0.000026 | gp:[GI:7299821] [LN:AE003702] [AC:AE003702:AE002708] |
| | | | | | | | [GN:ems] [OR:Drosophila melanogaster] [SR:fruit fly] [DB:genpept- |
| | | | | | | | inv2] [DE:Drosophila melanogaster genomic scaffold |
| | | | | | | | 142000013386035 section 27 of 105, complete sequence.] [NT:ems |
| | | | | | | | gene product; Nucleotide sequence of the Celera] [LE:93327:94752] |
| | | | | | | | [RE:99461:95101] [DI:directJoin] |
| smorf014 | 130 | 803 | 282 | 93 | | | |
| smorf015 | 131 | 804 | 201 | 66 | | | |
| smorf017 | 132 | 805 | 267 | 88 | | | |
| smorf020 | 133 | 806 | 162 | 53 | | | |
| smorf021 | 134 | 807 | 324 | 107 | 110 | 0.0000032 | pir:[LN:T11679] [AC:T11679] [PN: protein SPBC21D10.07] |
| | | | | | | | [CL:Schizosaccharomyces pombe protein SPBC21D10.07] |
| | | | | | | | [OR:Schizosaccharomyces pombe] [DB:pir2] [MP:IIR]> |
| | | | | | | | gp:[GI:3560210] [LN:SPBC21D10] [AC:AL031536] [PN:protein] |
| | | | | | | | [GN:SPBC21D10.07] [OR:Schizosaccharomyces pombe] [SR:fission |
| | | | | | | | yeast] [DB:genpept-pln4] [DE:S.pombe chromosome II cosmid |
| | | | | | | | c21D10.] [NT:SPBC21D10.07, len:104] [LE:13696:13925] |
| | | | | | | | [RE:13866:14068] [DI:complementJoin] |
| smorf022 | 135 | 808 | 279 | 92 | 78 | 0.02 | gp:[GI:9366789] [LN:TBBCHR1A] [AC:AL359782] [PN: protein, |
| | | | | | | | CHR1.313.] [GN:CHR1.313] [OR:Trypanosoma brucei] [DB:genpept- |
| | | | | | | | htg24] [DE:Trypanosoma brucei chromosome 1 strain TREU927] |
| | | | | | | | [NT:CHR1.313, len = 189 aa, reasonable] [LE:682194] [RE:682763] |
| | | | | | | | [DI:direct] |
| smorf023 | 136 | 809 | 393 | 130 | 84 | 0.026 | pir:[LN:C48175] [AC:C48175] [PN: plasmid replication protein (fosB |
| | | | | | | | 3′region)] [CL:replication protein] [OR:Staphylococcus epidermidis] |
| | | | | | | | [DB:pir2] |
| smorf025 | 137 | 810 | 174 | 57 | | | |
| smorf026 | 138 | 811 | 225 | 74 | | | |
| smorf027 | 139 | 812 | 183 | 60 | | | |
| smorf029 | 140 | 813 | 138 | 45 | | | |
| smorf031 | 141 | 814 | 186 | 61 | | | |
| smorf033 | 142 | 815 | 114 | 37 | 98 | 0.00083 | gp:[GI:3864] [LN:SCKRS1] [AC:X56259] [PN:lysine-tRNA ligase] |
| | | | | | | | [GN:KRS1] [OR:Saccharomyces cerevisiae] [SR:baker's yeast] |
| | | | | | | | [DB:genpept-pln4] [EC:6.1.1.6] [DE:S.cerevisiae strain 7305b mutant |
| | | | | | | | KRS1 gene for lysyl-tRNA synthetase.] [SP:P15180] [LE:305] |
| | | | | | | | [RE:2080] [DI:direct] |
| smorf034 | 143 | 816 | 207 | 68 | | | |
| smorf036 | 144 | 817 | 183 | 60 | | | |
| smorf038 | 145 | 818 | 180 | 59 | | | |
| smorf039 | 146 | 819 | 318 | 105 | | | |
| smorf041 | 147 | 820 | 255 | 84 | | | |
| smorf042 | 148 | 821 | 135 | 44 | | | |
| smorf043 | 149 | 822 | 135 | 44 | | | |
| smorf045 | 150 | 823 | 189 | 62 | | | |
| smorf047 | 151 | 824 | 297 | 98 | | | |
| smorf048 | 152 | 825 | 165 | 54 | | | |
| smorf049 | 153 | 826 | 249 | 82 | | | |
| smorf050 | 154 | 827 | 300 | 99 | | | |
| smorf051 | 155 | 828 | 165 | 54 | | | |
| smorf052 | 156 | 829 | 210 | 69 | | | |
| smorf055 | 157 | 830 | 213 | 70 | 71 | 0.043 | pir:[LN:PQ0372] [AC:PQ0372:S18112] [PN:protein D] |
| | | | | | | | [OR:Clostridium butyricum] [DB:pir2] |
| smorf056 | 158 | 831 | 102 | 33 | | | |
| smorf058 | 159 | 832 | 117 | 38 | | | |
| smorf059 | 160 | 833 | 165 | 54 | | | |
| smorf060 | 161 | 834 | 171 | 56 | 53 | 0.02 | gp:[GI:5790213] [LN:AB031286] [AC:AB031286] [PN:NADH |
| | | | | | | | dehydrogenase subunit 4] [GN:ND4] [OR:Mitochondrion Taenia |
| | | | | | | | hydatigena] [SR:Taenia hydatigena bladder worm mitochondrion |
| | | | | | | | DNA] [DB:genpept-inv1] [DE:Taenia hydatigena mitochondrial DNA, |
| | | | | | | | NADH dehydrogenase subunit 4, tRNA-Gln, tRNA-Phe, tRNA-Met, |
| | | | | | | | ATPase subunit 6, and NADH dehydrogenase subunit 2.] [NT:] |
| | | | | | | | [LE:<1] [RE:486] [DI:direct] |
| smorf062 | 162 | 835 | 249 | 82 | 265 | 1.2E−22 | pir:[LN:S70302] [AC:S70302] [PN: protein YBL109w] [GN:YBL109w] |
| | | | | | | | [OR:Saccharomyces cerevisiae] [DB:pir2] [MP:2L] |
| smorf069 | 163 | 836 | 204 | 67 | | | |
| smorf071 | 164 | 837 | 120 | 39 | | | |
| smorf072 | 165 | 838 | 357 | 118 | 366 | 2.4E−33 | gp:[GI:14588910] [LN:SCCHRIII] |
| | | | | | | | [AC:X59720:S43845:S49180:S58084:S93798] [PN: protein] |
| | | | | | | | [OR:Saccharomyces cerevisiae] [SR:baker's yeast] [DB:genpept- |
| | | | | | | | pln4] [DE:S.cerevisiae chromosome III complete DNA sequence.] |
| | | | | | | | [NT:ORF YCL026c-b -strong similarity to FRM2] [LE:73405] |
| | | | | | | | [RE:73986] [DI:complement] |
| smorf073 | 166 | 839 | 156 | 51 | 81 | 0.0038 | sp:[LN:YEA3_SCHPO] [AC:O14068] |
| | | | | | | | [GN:SPAC2E 11.03C:SPAC1687.07] [OR:Schizosaccharomyces |
| | | | | | | | pombe] [SR:Fission yeast] [DE: 13.9 KDA PROTEIN C2E11.03C IN |
| | | | | | | | CHROMOSOME I] [SP:O14068] [DB:swissprot]>pir:[LN:T37750] |
| | | | | | | | [AC:T37750] [PN: protein SPAC1687.07] [GN:SPAC1687.07] |
| | | | | | | | [CL:Schizosaccharomyces pombe protein SPAC1687.07] |
| | | | | | | | [OR:Schizosaccharomyces pombe] [DB:pir2] [MP:1]> |
| | | | | | | | gp:[GI:4106661] [LN:SPAC1687] [AC:AL035064] |
| | | | | | | | [GN:SPAC1687.07] [OR:Schizosaccharomyces pombe] [SR:fission |
| | | | | | | | yeast] [DB:genpept-pln4] [DE:S.pombe chromosome I cosmid |
| | | | | | | | c1687] [NT:SPAC1687.07, len:124] [SP:O14068] [LE:10394] |
| | | | | | | | [RE:10768] [DI:direct]>gp:[GI:3395567] [LN:SPUNK5] |
| | | | | | | | [AC:AL031181] [GN:SPAC2E11.03c] [OR:Schizosaccharomyces |
| | | | | | | | pombe] [SR:fission yeast] [DB:genpept-pln4] [DE:S.pombe |
| | | | | | | | chromosome I cosmid c2E11.] [NT:SPAC2E11.03c, len:124aa] |
| | | | | | | | [SP:O14068] [LE:1909] [RE:2283] [DI:complement] |
| smorf074 | 167 | 840 | 189 | 62 | 83 | 0.0024 | sp:[LN:YEA3_SCHPO] [AC:O14068] |
| | | | | | | | [GN:SPAC2E11.03C:SPAC1687.07] [OR:Schizosaccharomyces |
| | | | | | | | pombe] [SR:Fission yeast] [DE:13.9 KDA PROTEIN C2E11.03C IN |
| | | | | | | | CHROMOSOME I] [SP:O14068] [DB:swissprot]>pir:[LN:T37750] |
| | | | | | | | [AC:T37750] [PN: protein SPAC1687.07] [GN:SPAC1687.07] |
| | | | | | | | [CL:Schizosaccharomyces pombe protein SPAC1687.07] |
| | | | | | | | [OR:Schizosaccharomyces pombe] [DB:pir2] [MP:1]> |
| | | | | | | | gp:[GI:4106661] [LN:SPAC1687] [AC:AL035064] |
| | | | | | | | [GN:SPAC1687.07] [OR:Schizosaccharomyces pombe] [SR:fission |
| | | | | | | | yeast] [DB:genpept-pln4] [DE:S.pombe chromosome I cosmid |
| | | | | | | | c1687.] [NT:SPAC1687.07, len:124] [SP:O14068] [LE:10394] |
| | | | | | | | [RE:10768] [DI:direct]>gp:[GI:3395567] [LN:SPUNK5] |
| | | | | | | | [AC:AL031181] [GN:SPAC2E11.03c] [OR:Schizosaccharomyces |
| | | | | | | | pombe] [SR:fission yeast] [DB:genpept-pln4] [DE:S.pombe |
| | | | | | | | chromosome I cosmid c2E11] [NT:SPAC2E11.03c, len:124aa] |
| | | | | | | | [SP:O14068] [LE:1909] [RE:2283] [DI:complement] |
| smorf081 | 168 | 841 | 219 | 72 | 163 | 3.9E−11 | gp:[GI:1870134] [LN:SCZ86109] [AC:Z86109] [PN:] |
| | | | | | | | [OR:Saccharomyces pastorianus] [DB:genpept-pln4] |
| | | | | | | | [DE:S.carlsbergensis 12 kb region of chromosome III.] [NT:similarity |
| | | | | | | | to yeast ORF YNL001w] [LE:9227] [RE:10387] [DI:direct] |
| smorf083 | 169 | 842 | 159 | 52 | 85 | ta 0.0026 | gp:[GI:13794283] [LN:AF083031] [AC:AF083031] [PN: protein |
| | | | | | | | [GN:orf176] [OR:Nucleomorph Guillardia theta] [SR:Guillardia theta] |
| | | | | | | | [DB:genpept-pln1] [DE:Guillardia theta nucleomorph chromosome 3, |
| | | | | | | | complete sequence.] [NT:overlaps trnL by 42 nucleotides at 5′end;] |
| | | | | | | | [LE:24683] [RE:25213] [DI:direct] |
| smorf086 | 170 | 843 | 153 | 50 | | | |
| smorf087 | 171 | 844 | 270 | 89 | | | |
| smorf089 | 172 | 845 | 171 | 56 | | | |
| smorf090 | 173 | 846 | 228 | 75 | 72 | 0.034 | pir:[LN:T41216] [AC:T41216] [PN:protein SPCC191.03c] |
| | | | | | | | [GN:SPCC191.03c] [CL:Schizosaccharomyces pombe protein |
| | | | | | | | SPCC191.03c] [OR:Schizosaccharomyces pombe] [DB:pir2] [MP:1]> |
| | | | | | | | gp:[GI:4678670] [LN:SPCC191] [AC:AL049644] |
| | | | | | | | [GN:SPCC191.03c] [OR:Schizosaccharomyces pombe] [SR:fission |
| | | | | | | | yeast] [DB:genpept-pln4] [DE:S.pombe chromosome III cosmid |
| | | | | | | | c191] [NT:SPCC191.03c, len:117, ORF] [LE:6748] [RE:7101] |
| | | | | | | | [DI:complement] |
| smorf091 | 174 | 847 | 363 | 120 | | | |
| smorf094 | 175 | 848 | 285 | 94 | 223 | 3.4E−18 | pir:[LN:S70302] [AC:S70302] [PN:protein YBL109w] [GN:YBL109w] |
| | | | | | | | [OR:Saccharomyces cerevisiae] [DB:pir2] [MP:2L] |
| smorf095 | 176 | 849 | 189 | 62 | | | |
| smorf099 | 177 | 850 | 228 | 75 | | | |
| smorf105 | 178 | 851 | 204 | 67 | | | |
| smorf106 | 179 | 852 | 177 | 58 | | | |
| smorf110 | 180 | 853 | 222 | 73 | 83 | 0.004 | sp:[LN:Y019_BORBU] [AC:O51051] [GN:BB0019] [OR:Borrelia |
| | | | | | | | burgdorferi] [SR:Lyme disease spirochete] [DE:PROTEIN BB0019] |
| | | | | | | | [SP:O51051] [DB:swissprot]>pir:[LN:C70102] [AC:C70102] [PN: |
| | | | | | | | protein BB0019] [OR:Borrelia burgdorferi] [SR:,Lyme disease |
| | | | | | | | spirochete] [DB:pir2]>gp:[GI:2687906] [LN:AE001116] |
| | | | | | | | [AC:AE001116:AE000783] [PN:B. burgdorferi coding region BB0019] |
| | | | | | | | [GN:BB0019] [OR:Borrelia burgdorferi] [SR:Lyme disease |
| | | | | | | | spirochete] [DB:genpept-bct1] [DE:Borrelia burgdorferi (section 2 of |
| | | | | | | | 70) of the complete genome.] [NT:Protein; identified by Glimmer;] |
| | | | | | | | [LE:2039] [RE:2551] [DI:complement] |
| smorf114 | 181 | 854 | 237 | 78 | | | |
| smorf115 | 182 | 855 | 279 | 92 | 71 | 0.044 | gp:[GI:7292124] [LN:AE003472] [AC:AE003472:AE002584] |
| | | | | | | | [GN:CG13919] [OR:Drosophila melanogaster] [SR:fruit fly] |
| | | | | | | | [DB:genpept-inv1] [DE:Drosophila melanogaster genomic scaffold |
| | | | | | | | 142000013386045 section 6 of 17, complete sequence.] |
| | | | | | | | [NT:CG13919 gene product] [LE:110844] [RE:111239] [DI:direct] |
| smorf116 | 183 | 856 | 186 | 61 | 68 | 0.0012 | gp:[GI:10178678] [LN:AF295546] [AC:AF295546] [PN:orf120] |
| | | | | | | | [GN:orf120] [OR:Mitochondrion Malawimonas jakobiformis] |
| | | | | | | | [SR:Malawimonas jakobiformis] [DB:genpept-inv3] |
| | | | | | | | [DE:Malawimonas jakobiformis mitochodrial DNA, complete |
| | | | | | | | genome.] [LE:12057] [RE:12419] [DI:complement] |
| smorf117 | 184 | 857 | 237 | 78 | | | |
| smorf128 | 185 | 858 | 135 | 44 | | | |
| smorf129 | 186 | 859 | 168 | 55 | | | |
| smorf132 | 187 | 860 | 234 | 77 | | | |
| smorf133 | 188 | 861 | 222 | 73 | 75 | 0.028 | pir:[LN:T15593] [AC:T15593] [PN:protein C24H10.3] |
| | | | | | | | [GN:C24H10.3] [CL:Caenorhabditis elegans protein C24H10.3] |
| | | | | | | | [OR:Caenorhabditis elegans] [DB:pir2]>gp:[GI:1065538] |
| | | | | | | | [LN:CELC24H10] [AC:U40423] [GN:C24H10.3] [OR:Caenorhabditis |
| | | | | | | | elegans] [SR:Caenorhabditis elegans strain=Bristol N2] [DB:genpept- |
| | | | | | | | inv3] [DE:Caenorhabditis elegans cosmid C24H10.] |
| | | | | | | | [LE:3212:3614:3711:4280] [RE:3405:3668:3761:4393] [DI:directJoin] |
| smorf134 | 189 | 862 | 183 | 60 | | | |
| smorf136 | 190 | 863 | 234 | 77 | | | |
| smorf138 | 191 | 864 | 261 | 86 | | | |
| smorf142 | 192 | 865 | 123 | 40 | | | |
| smorf143 | 193 | 866 | 198 | 65 | | | |
| smorf145 | 194 | 867 | 87 | 28 | | | |
| smorf146 | 195 | 868 | 156 | 51 | | | |
| smorf147 | 196 | 869 | 132 | 43 | | | |
| smorf149 | 197 | 870 | 186 | 61 | | | |
| smorf150 | 198 | 871 | 225 | 74 | | | |
| smorf152 | 199 | 872 | 213 | 70 | 164 | 6.1E−12 | sp:[LN:YAUE_SCHPO] [AC:Q10167] [GN:SPAC26A3.14C] |
| | | | | | | | [OR:Schizosaccharomyces pombe] [SR:Fission yeast] [DE: 8.2 KDA |
| | | | | | | | PROTEIN C26A3.14C IN CHROMOSOME I] [SP:Q10167] |
| | | | | | | | [DB:swissprot]>pir:[LN:T38402] [AC:T38402] [PN:protein |
| | | | | | | | SPAC26A3.14c] [GN:SPAC23A6. 14c:SPAC26A3.14c] |
| | | | | | | | [OR:Schizosaccharomyces pombe] [DB:pir2] [MP:1]> |
| | | | | | | | gp:[GI:1177361] [LN:SPAC26A3] [AC:Z69240] |
| | | | | | | | [GN:SPAC23A6.14c] [OR:Schizosaccharomyces pombe] [SR:fission |
| | | | | | | | yeast] [DB:genpept-pln4] [DE:S.pombe chromosome I cosmid 26A3.] |
| | | | | | | | [NT:SPAC23A6.14c, len:73] [SP:Q10167] [LE:32637:32826:32948] |
| | | | | | | | [RE:32766:32914:32950] [DI:complement Join] |
| smorf153 | 200 | 873 | 204 | 67 | | | |
| smorf155 | 201 | 874 | 366 | 121 | | | |
| smorf156 | 202 | 875 | 186 | 61 | | | |
| smorf157 | 203 | 876 | 171 | 56 | 57 | 0.042 | gp:[GI:13446760] [LN:AF319593] [AC:AF319593] [PN: ferredoxin] |
| | | | | | | | [GN:nbzJ] [OR:Pseudomonas putida] [DB:genpept-bct2] |
| | | | | | | | [DE:Pseudomonas putida plasmid pNB1 aminophenol operon |
| | | | | | | | repressor (nbzR)gene, complete cds; and aminophenol operon, |
| | | | | | | | complete sequence.] [NT:NbzJ] [LE:1059] [RE:1487] [DI:direct] |
| smorf158 | 204 | 877 | 306 | 101 | | | |
| smorf159 | 205 | 878 | 333 | 110 | | | |
| smorf160 | 206 | 879 | 213 | 70 | | | |
| smorf161 | 207 | 880 | 174 | 57 | | | |
| smorf162 | 208 | 881 | 258 | 85 | 118 | 0.00000072 | gp:[GI:2511678] [LN:MTAJ2019] [AC:AJ002019] [PN:cytochrome |
| | | | | | | | oxidase subunit 2] [GN:coxll] [OR:Mitochondrion Saccharomyces |
| | | | | | | | bayanus] [SR:Saccharomyces bayanus] [DB:genpept-pln3] |
| | | | | | | | [DE:Saccharomyces uvarum mitochondrial coxll gene, partial.] |
| | | | | | | | [LE:<1] [RE:>636] [DI:direct] |
| smorf163 | 209 | 882 | 285 | 94 | 146 | 5E−10 | pir:[LN:S62023] [AC:S62023] [PN: membrane protein YDR544c: |
| | | | | | | | protein D3703.5] [GN:YDR544c] [OR:Saccharomyces cerevisiae] |
| | | | | | | | [DB:pir2] [MP:4R]>gp:[GI:1165299] [LN:SCU43834] |
| | | | | | | | [AC:U43834:Z71256] [PN:Ydr544cp] [GN:YDR544C] |
| | | | | | | | [OR:Saccharomyces cerevisiae] [SR:baker's yeast]DB:genepept- |
| | | | | | | | pln4] [DE:Saccharomyces cerevisiae chromosome IV lambda 3073 |
| | | | | | | | and flankingregion extending into right telomere.] [NT:similar to 17.1 |
| | | | | | | | KD protein in PUR5] [LE:15357] [RE:15785] [DI:complement] |
| smorf164 | 210 | 883 | 225 | 74 | | | |
| smorf165 | 211 | 884 | 204 | 67 | | | |
| smorf166 | 212 | 885 | 153 | 50 | | | |
| smorf168 | 213 | 886 | 222 | 73 | | | |
| smorf169 | 214 | 887 | 198 | 65 | | | |
| smorf170 | 215 | 888 | 189 | 62 | | | |
| smorf173 | 216 | 889 | 297 | 98 | 77 | 0.015 | sp:[LN:YM04_PARTE] [AC:P15605] [OR:Paramecium tetraurelia] |
| | | | | | | | [DE: 18.8 KDA PROTEIN (ORF 4)] [SP:P15605] [DB:swissprot] |
| | | | | | | | >pir:[LN:S07729] [AC:S07729] [PN:protein 4] [CL:cytochrome-c |
| | | | | | | | oxidase chain III] [OR:mitochondrion Paramecium tetraurelia] |
| | | | | | | | [DB:pir2]>gp:[GI:13261] [LN:MIPAGEN] [AC:X15917] |
| | | | | | | | [OR:Mitochondrion Paramecium aurelia] [SR:Paramecium aurelia] |
| | | | | | | | [DB:genpept-inv4] [DE:Paramecium aurelia mitochondrial complete |
| | | | | | | | genome.] [NT:ORF4 protein (AA 1-156)] [SP:P15605] [LE:5873] |
| | | | | | | | [RE:6343] [DI:direct] |
| smorf174 | 217 | 890 | 318 | 105 | 75 | 0.016 | sp:[LN:VE5_HPV70] [AC:P50774] [GN:E5] [OR:Human |
| | | | | | | | papillomavirus type 70] [DE: E5 PROTEIN] [SP:P50774] |
| | | | | | | | [DB:swissprot]>gp:[GI:717157] [LN:HPU21941] [8AC:U21941] |
| | | | | | | | [GN:E5] [OR:Human papillomavirus type 70] [DB:genepept-vr12] |
| | | | | | | | [DE:Human papillomavirus type 70, complete genome.] [NT:Method: |
| | | | | | | | conceptual translation supplied by author.;] [LE:3909] [RE:4145] |
| | | | | | | | [DI:direct] |
| smorf175 | 218 | 891 | 198 | 65 | | | |
| smorf176 | 219 | 892 | 111 | 36 | | | |
| smorf177 | 220 | 893 | 111 | 36 | | | |
| smorf178 | 221 | 894 | 273 | 90 | | | |
| smorf179 | 222 | 895 | 156 | 51 | | | |
| smorf182 | 223 | 896 | 123 | 40 | | | |
| smorf183 | 224 | 897 | 381 | 126 | 359 | 3.7E−32 | gp:[GI:559926] [LN:SC6584] [AC:Z46255] [OR:Saccharomyces |
| | | | | | | | cerevisiae] [SR:baker's yeast] [DB:genpept-pln4] [DE:S.cerevisiae |
| | | | | | | | chromosome VI lambda clone.] [NT:cdc4, incomplete, len: 579, CAI, |
| | | | | | | | 0.15, CC4_YEAST] [SP:P07834] [LE:<1] [RE:1738] [DI: complement] |
| smorf185 | 225 | 898 | 102 | 33 | | | |
| smorf186 | 226 | 899 | 219 | 72 | 58 | 0.012 | pir:[LN:G72126] [AC:G72126] [PN: ct338 protein] [GN:CPn0036] |
| | | | | | | | [OR:Chlamydophila pneumoniae: Chlamydia pneumoniae] [DB:pir2]> |
| | | | | | | | :gp[GI:8978411] [LN:AP002545] |
| | | | | | | | [AC:AP002545:AB033780:AB033781:AB033792:AB033793:AB0337 |
| | | | | | | | 94:AB033795] [PN:CT338 protein] [GN:CPj0036] |
| | | | | | | | [OR:Chlamydophila pneumoniae J138] [SR:Chlamydophila |
| | | | | | | | pneumoniae J138 (strain:J138) DNA] [DB:genepept-bct2] |
| | | | | | | | [DE:Chlamydophila pneumoniae J 138 genomic DNA, complete |
| | | | | | | | sequence, section 1/4.] [LE:50673] [RE:51470] [DI:direct]> |
| | | | | | | | gp:[GI:4376290] [LN:AE001589] [AC:AE001589:AE001363] |
| | | | | | | | [PN:CT338 protein] [GN:CPn0036] [OR:Chlamydophila |
| | | | | | | | pneumoniae CWL029] [DB:genepept-bct1] [DE:Chlamydia |
| | | | | | | | pneumoniae section 5 of 103 of the complete genome.] [LE:1521] |
| | | | | | | | [RE:2318] [DI:direct] |
| smorf187 | 227 | 900 | 192 | 63 | | | |
| smorf188 | 228 | 901 | 144 | 47 | | | |
| smorf190 | 229 | 902 | 192 | 63 | | | |
| smorf191 | 230 | 903 | 219 | 72 | 85 | 0.0014 | pir:[LN:S78736] [AC:S78736] [PN:protein YOL013w-a] |
| | | | | | | | [OR:Saccharomyces cerevisiae] [DB:pir2] [MP:15L] |
| smorf192 | 231 | 904 | 180 | 59 | | | |
| smorf193 | 232 | 905 | 264 | 87 | | | |
| smorf194 | 233 | 906 | 189 | 62 | | | |
| smorf195 | 234 | 907 | 186 | 61 | | | |
| smorf196 | 235 | 908 | 108 | 35 | | | |
| smorf197 | 236 | 909 | 570 | 189 | 228 | 1E−18 | sp:[LN:YH17_YEAST] [AC:P38898] [GN:YHR217C] |
| | | | | | | | [OR:Saccharomyces cerevisiae] [SR:,Baker's yeast] [DE: 17.1 KDA |
| | | | | | | | PROTEIN IN PUR5 3′REGION] [SP:P38898] [DB:swissprot]> |
| | | | | | | | pir:[LN:S48998] [AC:S48998] [PN: protein YHR217c] |
| | | | | | | | [GN:YHR217c] [OR:Saccharomyces cerevisiae] [DB:pir2] [MP:8R]> |
| | | | | | | | gp:[GI:551324] [LN:YSCH9177] [AC:U00029:U00093] |
| | | | | | | | [PN:Yhr217cp] [GN:YHR217c] [OR:Saccharomyces cerevisiae] |
| | | | | | | | [SR:baker's yeast strain=S288C (AB972)] [DB:genpept-pln4] |
| | | | | | | | [DE:Saccharomyces cerevisiae chromosome VIII cosmid 9177.] |
| | | | | | | | [LE:50035] [RE:50496] [DI:complement] |
| smorf198 | 237 | 910 | 180 | 59 | | | |
| smorf199 | 238 | 911 | 228 | 75 | | | |
| smorf200 | 239 | 912 | 171 | 56 | | | |
| smorf203 | 240 | 913 | 228 | 75 | | | |
| smorf204 | 241 | 914 | 108 | 35 | | | |
| smorf205 | 242 | 915 | 93 | 30 | | | |
| smorf206 | 243 | 916 | 216 | 71 | | | |
| smorf209 | 244 | 917 | 186 | 61 | | | |
| smorf210 | 245 | 918 | 264 | 87 | 244 | 3.7E−20 | gp:[GI:600456] [LN:SC8224] [AC:Z46902:Z47047] [PN: aspartyl |
| | | | | | | | protease] [OR:Saccharomyces cerevisiae] [SR:baker's yeast] |
| | | | | | | | [DB:genpept-pln4] [DE:S.cerevisiae chromosome IX cosmid 8224 |
| | | | | | | | and right telomere.] [NT:YI8224.01c, orf similar to YAP3_YEAST |
| | | | | | | | P32329] [SP:P40583] [LE:<1] [RE:1178] [DI:complement] |
| smorf211 | 246 | 919 | 228 | 75 | | | |
| smorf213 | 247 | 920 | 333 | 110 | | | |
| smorf214 | 248 | 921 | 153 | 50 | | | |
| smorf215 | 249 | 922 | 216 | 71 | 114 | 0.0000066 | pir:[LN:T40160] [AC:T40160] [PN:conserved protein SPBC2G5.03] |
| | | | | | | | [GN:SPBC2G5.03] [CL:conserved protein MJ1157] |
| | | | | | | | [OR:Schizosaccharomyces pombe] [DB:pir2] [MP:2]> |
| | | | | | | | gp:[GI:3850068] [LN:SPBC2G5] [AC:AL033385] [PN: protein] |
| | | | | | | | [GN:SPBC2G5.03] [OR:Schizosaccharomyces pombe] [SR:fission |
| | | | | | | | yeast] [DB:genpept-pln4] [DE:S.pombe chromosome II cosmid |
| | | | | | | | c2G5.] [NT:SPBC2G5.03, len:334, SIMILARITY:Arabidopsis] |
| | | | | | | | [LE:6068] [RE:7075] [DI:direct] |
| smorf216 | 250 | 923 | 174 | 57 | | | |
| smorf218 | 251 | 924 | 186 | 61 | | | |
| smorf219 | 252 | 925 | 264 | 87 | 162 | 5.9E−11 | pir:[LN:T50056] [AC:T50056] [PN: protein SPAC1039.06 [imported]] |
| | | | | | | | [GN:SPAC1039.06] [OR:Schizosaccharomyces pombe] [DB:pir2] |
| | | | | | | | [MP:1]>gp:[GI:6594265] [LN:SPAC1039] [AC:AL133521] [PN: |
| | | | | | | | protein] [GN:SPAC1039.06] [OR:Schizosaccharomyces pombe] |
| | | | | | | | [SR:fission yeast] [DB:genpept-pln4] [DE: S.pombe chromosome I |
| | | | | | | | cosmid c1039.] [NT:SAPC1039.06, len:415, SIMILARITY:LOW to] |
| | | | | | | | [LE:16592] [RE:17839] [DI:direct] |
| smorf221 | 253 | 926 | 258 | 85 | 71 | 0.043 | gp:[GI:12000391] [LN:AY008837] [AC:AY008837] [PN:CGRA] |
| | | | | | | | [GN:cgrA] [OR:Aspergillus fumigatus] [DB:genpept-pln3] |
| | | | | | | | [DE:Aspergillus fumigatus CGRA (cgrA) mRNA, complete cds.] |
| | | | | | | | [LE:77] [RE:421] [DI:direct] |
| smorf222 | 254 | 927 | 330 | 109 | | | |
| smorf223 | 255 | 928 | 270 | 89 | | | |
| smorf224 | 256 | 929 | 183 | 60 | 59 | 0.035 | gp:[GI:12850680] [LN:AK013366] [AC:AK013366] [OR:Mus |
| | | | | | | | musculus] [SR:Mus musculus (strain:C57BL/6J) 10, 11 days embryo |
| | | | | | | | cDNA to mRNA] [DB:genpept-htc] [DE:Mus musculus 10, 11 days |
| | | | | | | | embryo cDNA, RIKEN full-length enrichedlibrary, clone:2810459H04, |
| | | | | | | | full insert sequence.] [NT:] [LE:489] [RE:<1141] [DI:direct] |
| smorf225 | 257 | 930 | 180 | 59 | 129 | 0.00000022 | gp:[GI:12718471] [LN:NCB18D24] [AC:AL513466] [PN:related to |
| | | | | | | | branched-chain alpha-ketoacid] [GN:B18D24.20] [OR:Neurospora |
| | | | | | | | crassa [DB:genpept-pln3] [DE:Neurospora crassa DNA linkage |
| | | | | | | | group V BAC contig B18D24.] [NT:similarity to branched-chain alpha- |
| | | | | | | | ketoacid] [LE:69224:69500:70465] [RE:69420:70290:70715] |
| | | | | | | | [DI:direct Join] |
| smorf227 | 258 | 931 | 213 | 70 | | | |
| smorf229 | 259 | 932 | 192 | 63 | | | |
| smorf230 | 260 | 933 | 186 | 61 | 186 | 5.9E−14 | gp:[GI:171846] [LN:YSCLIPOLC] [AC:L11999] [PN:lipoic acid |
| | | | | | | | synthase] [GN:LIP] [FN:lipoic acid biosynthesis] [OR:Saccharomyces |
| | | | | | | | cerevisiae] [SR:Saccharomyces cerevisiae DNA] [DB:genpept-pln4] |
| | | | | | | | [DE:Saccharomyces cerevisiae (clone pg189/ST3) lipoic acid |
| | | | | | | | synthase(LIP) gene, 5′end cds.] [LE:281] [RE:>1246] [DI:direct] |
| smorf231 | 261 | 934 | 186 | 61 | | | |
| smorf233 | 262 | 935 | 132 | 43 | | | |
| smorf234 | 263 | 936 | 237 | 78 | | | |
| smorf235 | 264 | 937 | 93 | 30 | | | |
| smorf236 | 265 | 938 | 240 | 79 | | | |
| smorf237 | 266 | 939 | 105 | 34 | | | |
| smorf238 | 267 | 940 | 177 | 58 | | | |
| smorf239 | 268 | 941 | 246 | 81 | | | |
| smorf240 | 269 | 942 | 171 | 56 | 68 | 0.044 | sp:[LN:Y070_NPVAC] [AC:P41470] [OR:Autographa californica |
| | | | | | | | nuclear polyhedrosis virus] [SR:,AcMNPV] [DE: 34.4 KDA PROTEIN |
| | | | | | | | IN LEF3-IAP2 INTERGENIC REGION]SP:P41470] [DB:swissprot]> |
| | | | | | | | pir:[LN:G72858] [AC:G72858] [PN:AcOrf-70 protein] [GN:AcOrf-70] |
| | | | | | | | [OR:Autographa californica nuclear polyhedrosis virus:AcMNPV] |
| | | | | | | | [DB:pir2]>gp:[GI:559139] [LN:L22858] [AC:L22858] [PN:AcOrf-70 |
| | | | | | | | peptide] [GN:AcOrf-70] [OR:Autographa californica |
| | | | | | | | nucleopolyhedrovirus] [DB:genpept-vrl2] [DE:Autographa californica |
| | | | | | | | nucleopolyhedrovirus clone C6, completegenome.] [NT:34408 Da |
| | | | | | | | primary translation product] [LE:60110] [RE:60982] [DI:direct] |
| smorf241 | 270 | 943 | 192 | 63 | | | |
| smorf242 | 271 | 944 | 222 | 73 | | | |
| smorf243 | 272 | 945 | 147 | 48 | | | |
| smorf244 | 273 | 946 | 129 | 42 | | | |
| smorf245 | 274 | 947 | 114 | 37 | | | |
| smorf249 | 275 | 948 | 246 | 81 | | | |
| smorf251 | 276 | 949 | 201 | 66 | 73 | 0.027 | gp:[GI:14574088] [LN:AC006630] [AC:AC006630] [PN: protein |
| | | | | | | | F14H12.7] [GN:F14H12.7] [OR:Caenorhabditis elegans] |
| | | | | | | | [DB:genpept-inv1] [DE:Caenorhabditis elegans cosmid F14H12, |
| | | | | | | | complete sequence.] [LE:28511:28770] [RE:28712:28867] |
| | | | | | | | [DI:complementJoin] |
| smorf252 | 277 | 950 | 225 | 74 | | | |
| smorf253 | 278 | 951 | 162 | 53 | | | |
| smorf254 | 279 | 952 | 147 | 48 | | | |
| smorf255 | 280 | 953 | 204 | 67 | | | |
| smorf256 | 281 | 954 | 222 | 73 | | | |
| smorf257 | 282 | 955 | 168 | 55 | | | |
| smorf258 | 283 | 956 | 258 | 85 | 118 | 0.00000046 | pir:[LN:S62023] [AC:S62023] [PN: membrane protein YDR544c: |
| | | | | | | | protein D3703.5] [GN:YDR544c] [OR:Saccharomyces cerevisiae] |
| | | | | | | | [DB:pir2] [MP:4R[>gp:[GI:1165299] [LN:SCU43834] |
| | | | | | | | [AC:U43834:Z71256] [PN:Ydr544cp] [GN:YDR544C] |
| | | | | | | | [OR:Saccharomyces cerevisiae] [SR:baker's yeast] [DB:genpept- |
| | | | | | | | pln4] [DE:Saccharomyces cerevisiae chromosome IV lambda 3073 |
| | | | | | | | and flankingregion extending into right telomere.] [NT:similar to |
| | | | | | | | 17.1 KD protein in PUR5] [LE:15357] [RE:15785] [DI:complement] |
| smorf260 | 284 | 957 | 255 | 84 | 62 | 0.0086 | pir:[LN:E70199] [AC:E70199] [PN:competence protein F homology] |
| | | | | | | | [OR:Borrelia burgdorferi] [SR:,Lyme disease spirochete] [DB:pir2]> |
| | | | | | | | gp:[GI:2688750] [LN:AE001179] [AC:AE001179:AE000783] |
| | | | | | | | [PN:competence protein F] [GN:BB0798] [OR:Borrelia burgdorferi] |
| | | | | | | | [SR:Lyme disease spirochete] [DE:genpept-bct1] [DE:Borrelia |
| | | | | | | | burgdorferi (section 65 of 70) of the complete genome.] [NT:similar |
| | | | | | | | to GB:M59751 SP:P31773 PID:1573409 percent] [LE:2702] |
| | | | | | | | [RE:3319] [DI:direct] |
| smorf262 | 285 | 958 | 165 | 54 | | | |
| smorf263 | 286 | 959 | 132 | 43 | | 108 | |
| smorf264 | 287 | 960 | 171 | 56 | | | |
| smorf265 | 288 | 961 | 177 | 58 | | | |
| smorf266 | 289 | 962 | 240 | 79 | 150 | 1.5E−09 | sp:[LN:YKW1_YEAST] [AC:P36032] [GN:YKL221W] |
| | | | | | | | [OR:Saccharomyces cerevisiae] [SR:,Baker's yeast] [DE: 52.3 KDA |
| | | | | | | | PROTEIN IN FRE2 5′REGION] [SP:P36032] [DB:swissprot]> |
| | | | | | | | pir:[LN:S38065] [AC:S38065:S38064:S43549:S44511:S46546] |
| | | | | | | | [PN: protein YKL221w: protein B473] [OR:Saccharomyces |
| | | | | | | | cerevisiae] [DB:pir2] [MP:11L]>gp:[GI:473128] [LN:SC5ORF] |
| | | | | | | | [AC:X75950] [OR:Saccharomyces cerevisiae] [SR:baker's yeast] |
| | | | | | | | [DB:genpept-pln4] [DE:S.cerevisiae sequence five orfs.] [NT:ORF4, |
| | | | | | | | B473] [SP:P36032] [LE:4955] [RE:6376] [DI:direct]>gp:[GI:486397] |
| | | | | | | | [LN:SCYKL221W] [AC:Z28221:Y13137] [OR:Saccharomyces |
| | | | | | | | cerevisiae] [SR:baker's yeast] [DB:genpept-pln4] [DE:S.cerevisiae |
| | | | | | | | chromosome XI reading frame ORF YKL221w.] [NT:ORF YKL221w] |
| | | | | | | | [SP:P36032] [LE:487] [RE:1908] [DI:direct] |
| smorf269 | 290 | 963 | 204 | 67 | | | |
| smorf270 | 291 | 964 | 129 | 42 | | | |
| smorf271 | 292 | 965 | 99 | 32 | | | |
| smorf272 | 293 | 966 | 195 | 64 | | | |
| smorf273 | 294 | 967 | 261 | 86 | 73 | 0.027 | gp:[GI:7293741] [LN:AE003515] [AC:AE003515:AE002602] |
| | | | | | | | [GN:CG14104] [OR:Drosophila melanogaster] [SR:fruit fly] |
| | | | | | | | [DB:genpept-inv2] [DE:Drosophila melanogaster genomic scaffold |
| | | | | | | | 142000013386050 section 53of 54, complete sequence.] |
| | | | | | | | [NT:CG14104 gene product] [LE:29172] [RE:29378] [DI:complement] |
| smorf275 | 295 | 968 | 252 | 83 | | | |
| smorf276 | 296 | 969 | 243 | 80 | | | |
| smorf278 | 297 | 970 | 153 | 50 | | | |
| smorf280 | 298 | 971 | 264 | 87 | | | |
| smorf281 | 299 | 972 | 321 | 106 | | | |
| smorf282 | 300 | 973 | 132 | 43 | | | |
| smorf284 | 301 | 974 | 186 | 61 | 97 | 0.000077 | sp:[LN:YE11_YEAST] [AC:P40097] [GN:YER181C] |
| | | | | | | | [OR:Saccharomyces cerevisiae] [SR:,Baker's yeast] [DE: 12.5 KDA |
| | | | | | | | PROTEIN IN ISC10 3'REGION] [SP:P40097] [DB:swissprot]> |
| | | | | | | | pir:[LN:S50684] [AC:S50684] [PN: protein YER181c] |
| | | | | | | | [GN:YER181c] [OR:Saccharomyces cerevisiae] [DB:pir2] [MP:5R]> |
| | | | | | | | gp:[GI:603422] [LN:SCE9163] |
| | | | | | | | [AC:U18922:L10718:L11229:U00092] [PN:Yer181cp] [GN:YER181C] |
| | | | | | | | [OR:Saccharomyces cerevisiae] [SR:baker's yeast] [DB:genpept- |
| | | | | | | | pln4] [DE:Saccharomyces cerevisiae chromosome V cosmids 9163 |
| | | | | | | | and 9132.] [LE:41824] [RE:42147] [DI:complement] |
| smorf285 | 302 | 975 | 204 | 67 | | | |
| smorf287 | 303 | 976 | 120 | 39 | | | |
| smorf289 | 304 | 977 | 147 | 48 | | | |
| smorf290 | 305 | 978 | 306 | 101 | 99 | 0.001 | pir:[LN:T31613] [AC:T31613] [PN: protein Y50E8A.i] [GN:Y50E8A.i] |
| | | | | | | | [OR:Caenorhabditis elegans] [DB:pir2] |
| smorf291 | 306 | 979 | 183 | 60 | | | |
| smorf293 | 307 | 980 | 183 | 60 | | | |
| smorf295 | 308 | 981 | 159 | 52 | | | |
| smorf296 | 309 | 982 | 168 | 55 | 54 | 0.019 | pir:[LN:T03893] [AC:T03893] [PN: protein C13D9.1] |
| | | | | | | | [OR:Caenorhabditis elegans] [DB:pir2] [MP:V]>gp:[GI:2291170] |
| | | | | | | | [LN:CELC13D9] [AC:AF016420] [GN:C13D9.1] [OR:Caenorhabditis |
| | | | | | | | elegans] [SR:Caenorhabditis elegans strain=Bristol N2] [DB:genpept- |
| | | | | | | | inv3] [DE:Caenorhabditis elegans cosmid C13D9.] |
| | | | | | | | [LE:35527:36131:36609:37235] [RE:35651:36559:36929:37592] |
| | | | | | | | [DI:direct Join] |
| smorf297 | 310 | 983 | 165 | 54 | | | |
| smorf299 | 311 | 984 | 210 | 69 | | | |
| smorf300 | 312 | 985 | 198 | 65 | | | |
| smorf304 | 313 | 986 | 321 | 106 | 114 | 0.000045 | pir:[LN:S51364] [AC:S51364:S34154] [PN:sperm tail-specific protein |
| | | | | | | | mst101(2)] [GN:mst101(2)] [OR:Drosophila hydei] [DB:pir2] |
| smorf305 | 314 | 987 | 135 | 44 | 93 | 0.0018 | gp:[GI:13374872] [LN:ATT6G21] [AC:AL589883] |
| | | | | | | | [PN:mannosyltransferase-like protein] [GN:At5g22130] |
| | | | | | | | [OR:Arabidopsis thaliana] [SR:thale cress] [DB:genpept-pln3] |
| | | | | | | | [DE:Arabidopsis thaliana DNA chromosome 5, BAC clone T6G21 |
| | | | | | | | (ESSAproject).] [NT:strong similarity to mannosyltransferase - |
| | | | | | | | Homo] [LE:105204:105650] [RE:105521:106194] [DI:complement |
| | | | | | | | Join] |
| smorf307 | 315 | 988 | 102 | 33 | | | |
| smorf308 | 316 | 989 | 147 | 48 | | | |
| smorf309 | 317 | 990 | 87 | 28 | | | |
| smorf311 | 318 | 991 | 237 | 78 | | | |
| smorf312 | 319 | 992 | 297 | 98 | | | |
| smorf314 | 320 | 993 | 243 | 80 | 96 | 0.000099 | gp:[GI:14028992] [LN:AC078891] [AC:AC078891] [PN: protein] |
| | | | | | | | [GN:OSJNBa0092N12.2] [OR:Oryza sativa] [DB:genpept-pln1] |
| | | | | | | | [DE:Oryza sativa chromosome 10 clone OSJNBa0092N12, complete |
| | | | | | | | sequence.] [LE:5755] [RE:6141] [DI:direct] |
| smorf316 | 321 | 994 | 147 | 48 | | | |
| smorf317 | 322 | 995 | 204 | 67 | | | |
| smorf320 | 323 | 996 | 219 | 72 | 83 | 0.045 | gp:[GI:9800258] [LN:AF232689] [AC:AF232689: AF046125: |
| | | | | | | | U50550:AF077758:U91788:AF133339:U57441:U57442] [PN:pR34] |
| | | | | | | | [GN:R34] [OR:rat cytomegalovirus Maastricht] [DB:genpept-vrl1] |
| | | | | | | | [DE:Rat cytomegalovirus Maastricht, complete genome.] [LE:27693] |
| | | | | | | | [RE:29993] [DI:direct] |
| smorf321 | 324 | 997 | 258 | 85 | 137 | 4.5E−09 | pir:[LN:S70302] [AC:S70302] [PN: protein YBL109w] [GN:YBL109w] |
| | | | | | | | [OR:Saccharomyces cerevisiae] [DB:pir2] [MP:2L] |
| smorf325 | 325 | 998 | 195 | 64 | 115 | 0.0000016 | pir:[LN:A83124] [AC:A83124] [PN: protein PA4182 [imported]] |
| | | | | | | | [GN:PA4182] [OR:Pseudomonas aeruginosa] [DB:pir2]> |
| | | | | | | | gp:[GI:9950391] [LN:AE004834] [AC:AE004834:AE004091] [PN: |
| | | | | | | | protein] [GN:PA4182] [OR:Pseudomonas aeruginosa] [DB:genpept- |
| | | | | | | | bct1] [DE:Pseudomonas aeruginosa PA01, section 395 of 529 of the |
| | | | | | | | completegenome.] [LE:9197] [RE:9835] [DI:direct] |
| smorf326 | 326 | 999 | 291 | 96 | 86 | 0.0012 | gp:[GI:4093023] [LN:AF070835] [AC:AF070835] [PN:NADH |
| | | | | | | | dehydrogenase subunit 4] [GN:ND4] [OR:Mitochondrion |
| | | | | | | | Mazamastrongylus odocoilei] [SR:Mazamastrongylus odocoilei] |
| | | | | | | | [DB:genpept-inv2] [DE:Mazamastrongylus odocoilei isolate mohb64 |
| | | | | | | | NADH dehydrogenasesubunit 4 (ND4) gene, mitochondrial gene |
| | | | | | | | encoding mitochondrialprotein, partial cds.] [LE:<1] [RE:463] |
| | | | | | | | [DI:direct] |
| smorf328 | 327 | 1000 | 147 | 48 | | | |
| smorf329 | 328 | 1001 | 264 | 87 | | | |
| smorf330 | 329 | 1002 | 225 | 74 | | | |
| smorf331 | 330 | 1003 | 567 | 188 | 326 | 4.2E−29 | pir:[LN:T40833] [AC:T40833] [PN:haloacid dehalogenase-like |
| | | | | | | | hydrolase] [GN:SPCC1020.07] [CL: protein b2690] |
| | | | | | | | [OR:Schizosaccharomyces pombe] [DB:pir2] [MP:3]> |
| | | | | | | | gp:[GI:3130050] [LN:SPCC1020] [AC:AL023518] [PN:haloacid |
| | | | | | | | dehalogenase-like hydrolas] [GN:SPCC1020.07] |
| | | | | | | | [OR:Schizosaccharomyces pombe] [SR:fission yeast] [DB:genpept- |
| | | | | | | | pln4] [DE:S.pombe chromosome III cosmid c1020.] |
| | | | | | | | [NT:SPCC1020.07, len:235,] [LE:18284:18913:19041] |
| | | | | | | | [RE:18855:18975:19083] [DI:complement Join] |
| smorf332 | 331 | 1004 | 219 | 72 | | | |
| smorf333 | 332 | 1005 | 129 | 42 | | | |
| smorf334 | 333 | 1006 | 186 | 61 | 55 | 0.044 | gp:[GI:5790238] [LN:AB031289] [AC:AB031289] [PN:ATPase |
| | | | | | | | subunit 6] [GN:ATP6] [OR:Mitochondrion Mesocestoides corti] |
| | | | | | | | [SR:Mesocestoides corti (isolate:tetrathyridium) mitochondrion DNA] |
| | | | | | | | [DB:genpept-inv1] [DE:Mesocestoides corti mitochondrial DNA, |
| | | | | | | | NADH dehydrogenase subunit4, tRNA-Gln, tRNA-Phe, tRNA-Met, |
| | | | | | | | ATPase subunit 6, and NADHdehydrogenase subunit 2.] [NT:] |
| | | | | | | | [LE:682] [RE:1194] [DI:direct] |
| smorf335 | 334 | 1007 | 366 | 121 | | | |
| smorf338 | 335 | 1008 | 213 | 70 | | | |
| smorf339 | 336 | 1009 | 129 | 42 | 73 | 0.027 | pir:[LN:T28394] [AC:T28394] [PN: protein MSV234 [imported]] |
| | | | | | | | [OR:Melanoplus sanguinipes entomopoxvirus] [DB:pir2]> |
| | | | | | | | gp:[GI:4049784] [LN:AF063866] [AC:AF063866] [PN:ORF MSV234 |
| | | | | | | | hypthetical protein] [GN:MSV234] [OR:Melanoplus sanguinipes |
| | | | | | | | entomopoxvirus] [DB:genpept-vrl1] [De:Melanoplus sanguinipes |
| | | | | | | | entomopoxvirus, complete genome.] [LE:201477] [RE:201830] |
| | | | | | | | [DI:complement] |
| smorf341 | 337 | 1010 | 207 | 68 | | | |
| smorf342 | 338 | 1011 | 261 | 86 | 88 | 0.00069 | sp:[LN:YAYD_SCHPO] [AC:Q10220] [GN:SPAC4H3.13] |
| | | | | | | | [OR:Schizosaccharomyces pombe] [SR:,Fission yeast] [DE: 10.1 |
| | | | | | | | KDA PROTEIN C4H3.13 IN CHROMOSOME I] [SP:Q10220] |
| | | | | | | | [DB:swissprot]>pir:[LN:T38893] [AC:T38893] [PN: protein |
| | | | | | | | SPAC4H3.13] [GN:SPAC4H3.13] [OR:Schizosaccharomyces |
| | | | | | | | pombe] [DB:pir2] [MP:1]>gp:[GI:1184026] [LN:SPAC4H3] |
| | | | | | | | [AC:Z69380] [PN: protein] [GN:SPAC4H3.13] |
| | | | | | | | [OR:Schizosaccharomyces pombe] [SR:fission yeast] [DB:genpept- |
| | | | | | | | pln4] [DE:S.pombe chromosome I cosmid c4H3.] [NT:SPAC4H3.13, |
| | | | | | | | len:88] [SP:Q10220] [LE:31154:31263] [RE:31185:31497] |
| | | | | | | | [DI:directJoin] |
| smorf343 | 339 | 1012 | 243 | 80 | | | |
| smorf344 | 340 | 1013 | 231 | 76 | 139 | 2.7E−09 | sp:[LN:YH17_YEAST] [AC:P38898] [GN:YHR217C] |
| | | | | | | | [OR:Saccharomyces cerevisiae] [SR:,Baker's yeast] [DE: 17.1 KDA |
| | | | | | | | PROTEIN IN PUR5 3'REGION] [SP:P38898] [DB:swissprot]> |
| | | | | | | | pir:[LN:S48998] [AC:S48998] [PN: protein YHR217c] |
| | | | | | | | [GN:YHR217c] [OR:Saccharomyces cerevisiae] [DB:pir2] [MP:8R]> |
| | | | | | | | gp:[GI:551324] [LN:YSCH9177] [AC:U00029:U00093] |
| | | | | | | | [PN:Yhr217cp] [GN:YHR217c] [OR:Saccharomyces cerevisiae] |
| | | | | | | | [SR:baker's yeast strain=S288C (AB972)] [DB:genpept-pln4] |
| | | | | | | | [DE:Saccharomyces cerevisiae chromosome VIII cosmid 9177.] |
| | | | | | | | [LE:50035] [RE:50496] [DI:complement] |
| smorf345 | 341 | 1014 | 462 | 153 | 305 | 7E−27 | sp:[LN:YH17_YEAST] [AC:P38898] [GN:YHR217C] |
| | | | | | | | [OR:Saccharomyces cerevisiae] [SR:,Baker's yeast] [DE: 17.1 KDA |
| | | | | | | | PROTEIN IN PUR5 3′REGION] [SP:P38898] [DB:swissprot]> |
| | | | | | | | pir:[LN:S48998] [AC:S48998] [PN: protein YHR217c] |
| | | | | | | | [GN:YHR217c] [OR:Saccharomyces cerevisiae] [DB:pir2] [MP:8R]> |
| | | | | | | | gp:[GI:551324] [LN:YSCH9177] [AC:U00029:U00093] |
| | | | | | | | [PN:Yhr217cp] [GN:YHR217c] [OR:Saccharomyces cerevisiae] |
| | | | | | | | [SR:baker's yeast strain=S288C (AB972)] [DB:genpept-pln4] |
| | | | | | | | [DE:Saccharomyces cerevisiae chromosome VIII cosmid 9177.] |
| | | | | | | | [LE:50035] [RE:50496] [DI:complement] |
| smorf346 | 342 | 1015 | 168 | 55 | | | |
| smorf347 | 343 | 1016 | 219 | 72 | 147 | 3.9E−10 | pir:[LN:S70302] [AC:S70302] [PN: protein YBL109w] [GN:YBL109w] |
| | | | | | | | [OR:Saccharomyces cerevisiae] [DB:pir2] [MP:2L] |
| smorf348 | 344 | 1017 | 180 | 59 | | | |
| smorf349 | 345 | 1018 | 174 | 57 | | | |
| smorf351 | 346 | 1019 | 198 | 65 | | | |
| smorf353 | 347 | 1020 | 159 | 52 | | | |
| smorf354 | 348 | 1021 | 132 | 43 | | | |
| smorf357 | 349 | 1022 | 186 | 61 | 58 | 0.028 | sp:[LN:ATPD_CYAPA] [AC:P48082] [GN:ATPD] [OR:Cyanophora |
| | | | | | | | paradoxa] [EC:3.6.1.34] [DE:ATP SYNTHASE DELTA CHAIN,] |
| | | | | | | | [SP:P48082] [DB:swissport]>pir:[LN:T06911] [AC:T06911] [PN:H+- |
| | | | | | | | transporting ATP synthase delta chain] [GN:atpD] [CL:H+- |
| | | | | | | | transporting ATP synthase delta chain] [OR:cyanelle Cyanophora |
| | | | | | | | paradoxa] [EC:3.6.1.34] [DB:pir2]>gp:[GI:1016167] [LN:CPU30821] |
| | | | | | | | [AC:U30821] [PN:delta subunit of F1 portion of ATP synthase] |
| | | | | | | | [GN:atpD] [OR:Cyanelle Cyanophora paradoxa] [SR:Cyanophora |
| | | | | | | | paradoxa] [DB:genpept-pln3] [DE:Cyanophora paradoxa cyanelle, |
| | | | | | | | complete genome.] [LE:72231] [RE:72791] [DI:complement] |
| smorf358 | 350 | 1023 | 174 | 57 | | | |
| smorf359 | 351 | 1024 | 207 | 68 | | | |
| smorf360 | 352 | 1025 | 177 | 58 | | | |
| smorf361 | 353 | 1026 | 66 | 21 | | | |
| smorf362 | 354 | 1027 | 237 | 78 | | | |
| smorf364 | 355 | 1028 | 63 | 20 | | | |
| smorf365 | 356 | 1029 | 93 | 30 | | | |
| smorf366 | 357 | 1030 | 87 | 28 | | | |
| smorf367 | 358 | 1031 | 261 | 86 | | | |
| smorf368 | 359 | 1032 | 168 | 55 | | | |
| smorf369 | 360 | 1033 | 102 | 33 | | | |
| smorf370 | 361 | 1034 | 108 | 35 | | | |
| smorf371 | 362 | 1035 | 108 | 35 | | | |
| smorf372 | 363 | 1036 | 198 | 65 | 80 | 0.0067 | pir:[LN:G72580] [AC:G72580] [PN: protein APE1926] |
| | | | | | | | [GN:APE1926] [OR:Aeropyrum pernix] [DB:pir2]>gp:[GI:5105619] |
| | | | | | | | [LN:AP000062] [AC:AP000062:BA000002] [PN:155aa long protein] |
| | | | | | | | [GN:APE1926] [OR:Aeropyrum pernix] [SR:Aeropyrum pernix |
| | | | | | | | (strain:K1) DNA] [DB:genpept-bct2] [DE:Aeropyrum pernix genomic |
| | | | | | | | DNA, section 5/7.] [LE:233088] [RE:233555] [DI:direct] |
| smorf373 | 364 | 1037 | 255 | 84 | | | |
| smorf374 | 365 | 1038 | 189 | 62 | 71 | 0.043 | pir:[LN:I48773] [AC:I48773:I48774:I48772] [PN:chloride channel, |
| | | | | | | | skeletal muscle] [GN:c1c-1] [CL:CBS homology] [OR:Mus musculus |
| | | | | | | | domesticus] [SR:western European house mouse] [DB:pir2] |
| smorf375 | 366 | 1039 | 108 | 35 | | | |
| smorf376 | 367 | 1040 | 60 | 19 | | | |
| smorf377 | 368 | 1041 | 69 | 22 | | | |
| smorf378 | 369 | 1042 | 66 | 21 | | | |
| smorf379 | 370 | 1043 | 66 | 21 | | | |
| smorf380 | 371 | 1044 | 141 | 46 | | | |
| smorf381 | 372 | 1045 | 117 | 38 | 85 | 0.0014 | gp:[GI:15028169] [LN:AY046034] [AC:AY046034] [PN: 5.8S |
| | | | | | | | ribosomal RNA protein] [GN:F23H14.12/At2g01020] |
| | | | | | | | [OR:Arabidopsis thaliana] [SR:thale cress] [DB:genpept-pln3] |
| | | | | | | | [DE:Arabidopsis thaliana 5.8S ribosomal RNA |
| | | | | | | | protein(F23H14.12/At2g01020) mRNA, complete cds.] [LE:38] |
| | | | | | | | [RE:280] [DI:direct] |
| smorf383 | 373 | 1046 | 54 | 17 | | | |
| smorf384 | 374 | 1047 | 99 | 32 | | | |
| smorf385 | 375 | 1048 | 69 | 22 | | | |
| smorf386 | 376 | 1049 | 123 | 40 | | | |
| smorf387 | 377 | 1050 | 141 | 46 | | | |
| smorf388 | 378 | 1051 | 132 | 43 | 114 | 0.0000047 | gp:[GI:7320865] [LN:HSA276485] [AC:AJ276485] [PN: integral] |
| | | | | | | | membrane transporter protein] [GN:LC27] [OR:Homo sapiens] |
| | | | | | | | [SR:human] [DB:genpept-pri11] [DE:Homo sapiens mRNA for |
| | | | | | | | integral membrane transporterprotein (LC27 gene).] [LE:204] |
| | | | | | | | [RE:1055] [DI:direct] |
| smorf389 | 379 | 1052 | 114 | 37 | | | |
| smorf390 | 380 | 1053 | 123 | 40 | | | |
| smorf391 | 381 | 1054 | 78 | 25 | | | |
| smorf393 | 382 | 1055 | 120 | 39 | | | |
| smorf394 | 383 | 1056 | 234 | 77 | | | |
| smorf395 | 384 | 1057 | 69 | 22 | | | |
| smorf396 | 385 | 1058 | 102 | 33 | | | |
| smort397 | 386 | 1059 | 156 | 51 | | | |
| smorf399 | 387 | 1060 | 120 | 39 | | | |
| smorf400 | 388 | 1061 | 201 | 66 | | | |
| smorf401 | 389 | 1062 | 66 | 21 | | | |
| smorf402 | 390 | 1063 | 99 | 32 | | | |
| smorf403 | 391 | 1064 | 132 | 43 | | | |
| smorf404 | 392 | 1065 | 81 | 26 | | | |
| smorf405 | 393 | 1066 | 219 | 72 | | | |
| smorf406 | 394 | 1067 | 117 | 38 | | | |
| smorf407 | 395 | 1068 | 90 | 29 | | | |
| smorf408 | 396 | 1069 | 135 | 44 | 132 | 1.5E−08 | gp:[GI:7144507] [LN:APU12823] [AC:U12823] [PN:hemolysin] |
| | | | | | | | [FN:potential virulence factor] [OR:Acanthamoeba polyphaga] |
| | | | | | | | [DB:genpept-inv3] [DE:Acanthamoeba polyphaga CDC:0187:1 |
| | | | | | | | hemolysin mRNA, complete cds.] [NT:proposed start codon is CTG] |
| | | | | | | | [LE:32] [RE:376] [DI:direct] |
| smorf409 | 397 | 1070 | 261 | 86 | 71 | 0.043 | sp:[LN:CH10_STRAL] [AC:Q00769] [GN:GROES] [OR:Streptomyces |
| | | | | | | | albus G] [DE:10 KDA CHAPERONIN (PROTEIN CPN10) PROTEIN |
| | | | | | | | GROES)] [SP:Q00769] [DB:swissprot]>gp:[GI:295176] |
| | | | | | | | [LN:STMGROELX] [AC:M76657] [PN:GROES protein] [GN:GROES] |
| | | | | | | | [OR:Streptomyces albus] [SR:Streptomyces albus (strain G) DNA] |
| | | | | | | | [DB:genpept-bct4] [DE:Streptomyces albus GROES (GROES) gene, |
| | | | | | | | complete cds; GROEL1(GROEL1) gene, complete cds.] [LE:101] |
| | | | | | | | [RE:409] [DI:direct] |
| smorf410 | 398 | 1071 | 141 | 46 | | | |
| smorf411 | 399 | 1072 | 75 | 24 | | | |
| smorf412 | 400 | 1073 | 57 | 18 | | | |
| smorf413 | 401 | 1074 | 252 | 83 | | | |
| smorf414 | 402 | 1075 | 78 | 25 | | | |
| smorf415 | 403 | 1076 | 108 | 35 | | | |
| smorf416 | 404 | 1077 | 60 | 19 | | | |
| smorf417 | 405 | 1078 | 159 | 52 | | | |
| smorf418 | 406 | 1079 | 69 | 22 | | | |
| smorf419 | 407 | 1080 | 159 | 52 | | | |
| smorf420 | 408 | 1081 | 57 | 18 | | | |
| smorf422 | 409 | 1082 | 141 | 46 | | | |
| smorf423 | 410 | 1083 | 60 | 19 | | | |
| smorf424 | 411 | 1084 | 78 | 25 | | | |
| smorf425 | 412 | 1085 | 54 | 17 | | | |
| smorf426 | 413 | 1086 | 72 | 23 | | | |
| smorf427 | 414 | 1087 | 120 | 39 | | | |
| smorf428 | 415 | 1088 | 90 | 29 | | | |
| smorf429 | 416 | 1089 | 75 | 24 | | | |
| smorf430 | 417 | 1090 | 111 | 36 | | | |
| smorf431 | 418 | 1091 | 162 | 53 | | | |
| smorf432 | 419 | 1092 | 60 | 19 | | | |
| smorf433 | 420 | 1093 | 81 | 26 | | | |
| smorf434 | 421 | 1094 | 60 | 19 | | | |
| smorf435 | 422 | 1095 | 117 | 38 | | | |
| smorf436 | 423 | 1096 | 153 | 50 | 183 | 6E−14 | pir:[LN:T02955] [AC:T02955] [PN: cytochrome P450 |
| | | | | | | | monooxygenase] [OR:Zea mays] [SR:,maize] [DB:pir2]> |
| | | | | | | | gp:[GI:2995384] [LN:ZMAJ4810] [AC:AJ004810] [PN:cytochrome |
| | | | | | | | P450 monooxygenase] [OR:Zea mays] [DB:genpept-pln4] [DE:Zea |
| | | | | | | | mays mays mRNA for cytochrome P450 monooxygenase, partial.] |
| | | | | | | | [LE:156] [RE:>966] [DI:direct] |
| smorf437 | 424 | 1097 | 117 | 38 | | | |
| smorf438 | 425 | 1098 | 135 | 44 | | | |
| smorf440 | 426 | 1099 | 84 | 27 | | | |
| smorf441 | 427 | 1100 | 90 | 29 | | | |
| smorf442 | 428 | 1101 | 156 | 51 | 71 | 0.043 | pir:[LN:E71245] [AC:E71245] [PN: protein PHS003] [GN:PHS003] |
| | | | | | | | [OR:Pyrococcus horikoshii] [DB:pir2]>gp:[GI:3256609] |
| | | | | | | | [LN:AP000001] [AC:AP000001: AB009465: AB009464: AB009466: |
| | | | | | | | AB009467: AB009468: AB009469] [PN:52aa long protein] |
| | | | | | | | [GN:PHS003] [OR:Pyrococcus horikoshii] [SR:Pyrococcus horikoshii |
| | | | | | | | (strain:OT3) DNA] [DB:genpept-bct2] [DE:Pyrococcus horikoshii OT3 |
| | | | | | | | genomic DNA, 1-287000 nt. position (1/7).] [NT:motif=ATP/GTP- |
| | | | | | | | binding site motif A (P-loop)] [LE:195076] [RE:195234] [DI:direct] |
| smorf443 | 429 | 1102 | 435 | 144 | 104 | 0.00026 | gp:[GI:13400109] [LN:RNU77931] [AC:U77931] [PN:rRNA promoter |
| | | | | | | | binding protein] [OR:Rattus norvegicus] [SR:Norway rat] |
| | | | | | | | [DB:genpept-rod2] [DE:Rattus norvegicus rRNA promoter binding |
| | | | | | | | protein mRNA, complete cds.] [NT:similar to 28S ribosomal RNA] |
| | | | | | | | [LE:147] [RE:1034] [DI:direct] |
| smorf444 | 430 | 1103 | 117 | 38 | | | |
| smorf445 | 431 | 1104 | 75 | 24 | | | |
| smorf446 | 432 | 1105 | 54 | 17 | 88 | 0.0034 | gp:[GI:13359451] [LN:AB049723] [AC:AB049723] [PN: senescence- |
| | | | | | | | associated protein] [GN:ssa-13] [OR:Pisum sativum] [SR:Pisum |
| | | | | | | | sativum (cultivar:Ichihara wase) immatured pods pods cDNA t] |
| | | | | | | | [DB:genpept-pln1] [DE:Pisum sativum ssa-13 mRNA for |
| | | | | | | | senescence-associated protein, partial cds.] [LE:<117] [RE:965] |
| | | | | | | | [DI:direct] |
| smorf447 | 433 | 1106 | 87 | 28 | | | |
| smorf448 | 434 | 1107 | 96 | 31 | 144 | 2.IE−09 | gp:[GI:13359451] [LN:AB049723] [AC:AB049723] [PN: senescence- |
| | | | | | | | associated protein] [GN:ssa-13] [OR:Pisum sativum] [SR:Pisum |
| | | | | | | | sativum (cultivar:Ichihara wase) immatured pods pods cDNA t] |
| | | | | | | | [DB:genpept-pln1] [DE:Pisum sativum ssa-13 mRNA for |
| | | | | | | | senescence-associated protein, partial cds.] [LE:<117] [RE:965] |
| | | | | | | | [DI:direct] |
| smorf449 | 435 | 1108 | 63 | 20 | | | |
| smorf450 | 436 | 1109 | 57 | 18 | | | |
| smorf451 | 437 | 1110 | 135 | 44 | 92 | 0.0032 | pir:[LN:T02995] [AC:T02995] [PN:unspecific monooxygenase: |
| | | | | | | | cytochrome P450 homolog TBP] [GN:cTBP] [OR:Nicotiana tabacum] |
| | | | | | | | [SR:,common tobacco] [EC:1.14.14.1] [DB:pir2]>gp:[GI:1545805] |
| | | | | | | | [LN:D64052] [AC:D64052] [PN:cytochrome P450 like_TBP] |
| | | | | | | | [GN:cTBP] [OR:Nicotiana tabacum] [SR:Nicotiana tabacum |
| | | | | | | | (strain:Bright Yellow 2) cDNA to mRNA] [DB:genpept-pln3] |
| | | | | | | | [EC:1.14.14.1] [DE:Nicotiana tabacum mRNA for cytochrome P450 |
| | | | | | | | like_TBP, complete cds.] [LE:155] [RE:1747] [DI:direct] |
| smorf452 | 438 | 1111 | 129 | 42 | 95 | 0.00058 | gp:[GI:13359451] [LN:AB049723] [AC:AB049723] [PN: senescence- |
| | | | | | | | associated protein] [GN:ssa-13] [OR:Pisum sativum] [SR:Pisum |
| | | | | | | | sativum (cultivar:Ichihara wase) immatured pods pods cDNA t] |
| | | | | | | | [DB:genpept-pln1] [DE:Pisum sativum ssa-13 mRNA for senescence- |
| | | | | | | | associated protein, partial cds.] [LE:<117] [RE:965] [DI:direct] |
| smorf453 | 439 | 1112 | 66 | 21 | | | |
| smorf454 | 440 | 1113 | 87 | 28 | | | |
| smorf455 | 441 | 1114 | 60 | 19 | | | |
| smorf456 | 442 | 1115 | 81 | 26 | 93 | 0.00088 | pir:[LN:T02955] [AC:T02955] [PN: cytochrome P450 |
| | | | | | | | monooxygenase] [OR:Zea mays] [SR:,maize] [DB:pir2]> |
| | | | | | | | gp:[GI:2995384] [LN:ZMAJ4810] [AC:AJ004810] [PN:cytochrome |
| | | | | | | | P450 monooxygenase] [OR:Zea mays] [DB:genpept-pln4] [DE:Zea |
| | | | | | | | mays mays mRNA for cytochrome P450 monooxygenase, partial.] |
| | | | | | | | [LE:156] [RE:>966] [DI:direct] |
| smorf457 | 443 | 1116 | 168 | 55 | 120 | 0.00000028 | pir:[LN:G81737] [AC:G81737] [PN: protein TC0130 [imported]] |
| | | | | | | | [GN:TC0130] [OR:Chlamydia muridarum:Chlamydia trachomatis |
| | | | | | | | MoPn] [DB:pir2] |
| smorf458 | 444 | 1117 | 57 | 18 | | | |
| smorf459 | 445 | 1118 | 66 | 21 | | | |
| smorf460 | 446 | 1119 | 57 | 18 | | | |
| smorf461 | 447 | 1120 | 78 | 25 | | | |
| smorf462 | 448 | 1121 | 108 | 35 | 76 | 0.022 | pir:[LN:A35664] [AC:A35664] [PN:Ppol endonuclease] |
| | | | | | | | [OR:Physarum polycephalum] [DB:pir2] |
| smorf463 | 449 | 1122 | 60 | 19 | | | |
| smorf464 | 450 | 1123 | 156 | 51 | | | |
| smorf465 | 451 | 1124 | 57 | 18 | | | |
| smorf466 | 452 | 1125 | 114 | 37 | | | |
| smorf467 | 453 | 1126 | 87 | 28 | | | |
| smorf468 | 454 | 1127 | 204 | 67 | 153 | 1.7E−10 | pir:[LN:T02955] [AC:T02955] [PN: cytochrome P450 |
| | | | | | | | monooxygenase] [OR:Zea mays] [SR:,maize] [DB:pir2]> |
| | | | | | | | gp:[GI:2995384] [LN:ZMAJ4810] [AC:AJ004810] [PN:cytochrome |
| | | | | | | | P450 monooxygenase] [OR:Zea mays] [DB:genpept-pln4] [DE:Zea |
| | | | | | | | mays mays mRNA for cytochrome P450 monooxygenase, partial.] |
| | | | | | | | [LE:156] [RE:>966] [DI:direct] |
| smorf469 | 455 | 1128 | 159 | 52 | 204 | 6E−16 | gp:[GI:5531330] [LN:PAM243883] [AC:AJ243883] [PN: transcription |
| | | | | | | | factor] [GN:Pa-en1] [FN: role in segmentation and neurogenesis] |
| | | | | | | | [OR:Periplaneta americana] [SR:American cockroach] [DB:genpept- |
| | | | | | | | inv4] [DE:Periplaneta americana mRNA for transcription factor(Pa- |
| | | | | | | | en1 gene).] [LE:154] [RE:1155] [DI:direct] |
| smorf470 | 456 | 1129 | 78 | 25 | | | |
| smorf471 | 457 | 1130 | 147 | 48 | | | |
| smorf472 | 458 | 1131 | 78 | 25 | | | |
| smorf473 | 459 | 1132 | 225 | 74 | 120 | 0.0000011 | gp:[GI:13400109] [LN:RNU77931] [AC:U77931] [PN:rRNA promoter |
| | | | | | | | binding protein] [OR:Rattus norvegicus] [SR:Norway rat] |
| | | | | | | | [DB:genpept-rod2] [DE:Rattus norvegicus rRNA promoter binding |
| | | | | | | | protein mRNA, complete cds.] [NT:similar to 28S ribosomal RNA] |
| | | | | | | | [LE:147] [RE:1034] [DI:direct] |
| smorf474 | 460 | 1133 | 93 | 30 | | | |
| smorf475 | 461 | 1134 | 63 | 20 | | | |
| smorf476 | 462 | 1135 | 111 | 36 | | | |
| smorf477 | 463 | 1136 | 54 | 17 | | | |
| smorf478 | 464 | 1137 | 174 | 57 | | | |
| smorf479 | 465 | 1138 | 102 | 33 | 108 | 0.000021 | gp:[GI:13359451] [LN:AB049723] [AC:AB049723] [PN: senescence- |
| | | | | | | | associated protein] [GN:ssa-13] [OR:Pisum sativum] [SR:Pisum |
| | | | | | | | sativum (cultivar:Ichihara wase) immatured pods pods cDNA t] |
| | | | | | | | [DB:genpept-pln1] [DE:Pisum sativum ssa-13 mRNA for senescence- |
| | | | | | | | associated protein, partial cds.] [LE:<117] [RE:965] [DI:direct] |
| smorf480 | 466 | 1139 | 93 | 30 | | | |
| smorf481 | 467 | 1140 | 258 | 85 | 125 | 0.00000028 | gp:[GI:13359451] [LN:AB049723] [AC:AB049723] [PN: senescence- |
| | | | | | | | associated protein] [GN:ssa-13] [OR:Pisum sativum] [SR:Pisum |
| | | | | | | | sativum (cultivar:Ichihara wase) immatured pods pods cDNA t] |
| | | | | | | | [DB:genpept-pln 1] [DE:Pisum sativum ssa-13 mRNA for senescence- |
| | | | | | | | associated protein, partial cds.] [LE:<117] [RE:965] [DI:direct] |
| smorf482 | 468 | 1141 | 60 | 19 | | | |
| smorf484 | 469 | 1142 | 174 | 57 | | | |
| smorf485 | 470 | 1143 | 111 | 36 | | | |
| smorf486 | 471 | 1144 | 120 | 39 | | | |
| smorf487 | 472 | 1145 | 213 | 70 | | | |
| smorf488 | 473 | 1146 | 177 | 58 | | | |
| smorf489 | 474 | 1147 | 174 | 57 | | | |
| smorf490 | 475 | 1148 | 102 | 33 | 104 | 0.000059 | gp:[GI:13359451] [LN:AB049723] [AC:AB049723] [PN: senescence- |
| | | | | | | | associated protein] [GN:ssa-13] [OR:Pisum sativum] [SR:Pisum |
| | | | | | | | sativum (cultivar:Ichihara wase) immatured pods pods cDNA t] |
| | | | | | | | [DB:genpept-pln1] [DE:Pisum sativum ssa-13 mRNA for senescence- |
| | | | | | | | associated protein, partial cds.] [LE:<117] [RE:965] [DI:direct] |
| smorf491 | 476 | 1149 | 159 | 52 | | | |
| smorf492 | 477 | 1150 | 78 | 25 | | | |
| smorf493 | 478 | 1151 | 93 | 30 | | | |
| smorf495 | 479 | 1152 | 264 | 87 | | | |
| smorf496 | 480 | 1153 | 195 | 64 | | | |
| smorf497 | 481 | 1154 | 273 | 90 | 78 | 0.037 | gp:[GI:7296162] [LN:AE003588] [AC:AE003588:AE002638] |
| | | | | | | | [GN:CG15880] [OR:Drosophila melanogaster] [SR:fruit fly] |
| | | | | | | | [DB:genpept-inv2] [DE:Drosophila melanogaster genomic scaffold |
| | | | | | | | 142000013386046 section 3of 16, complete sequence.] |
| | | | | | | | [NT:CG15880 gene product] [LE:196121:196319] |
| | | | | | | | [RE:196257:196973] [DI:complement Join] |
| smorf498 | 482 | 1155 | 177 | 58 | | | |
| smorf501 | 483 | 1156 | 306 | 101 | | | |
| smorf504 | 484 | 1157 | 222 | 73 | 74 | 0.021 | gp:[GI:3445246] [LN:CCO010256] [AC:AJ010256] [GN:nad5] |
| | | | | | | | [OR:Mitochondrion Chara corallina] [SR:Chara corallina] |
| | | | | | | | [DB:genpept-pln3] [DE:Chara corallina mitochondrial nad5 gene, |
| | | | | | | | partial.] [LE:<1] [RE:>290] [DI:direct] |
| smorf506 | 485 | 1158 | 159 | 52 | | | |
| smorf507 | 486 | 1159 | 189 | 62 | | | |
| smorf510 | 487 | 1160 | 276 | 91 | 79 | 0.0062 | pir:[LN:S32165] [AC:S32165] [PN: secretory protein] |
| | | | | | | | [OR:chloroplast Olisthodiscus luteus] [DB:pir2]>gp:[GI:288235] |
| | | | | | | | [LN:CHOLCCSA] [AC:Z21959] [PN: secretory protein] [GN:ORF 97] |
| | | | | | | | [OR:Plastid Heterosigma akashiwo] [SR:Heterosigma akashiwo] |
| | | | | | | | [DB:genpept-pln3] [DE:O.luteus chloroplast ORF 97 and bchl, and |
| | | | | | | | tRNA-Glu genes.] [NT:orf 97 is cotranscribed with ccsA. The] |
| | | | | | | | [LE:150] [RE:440] [DI:direct] |
| smorf512 | 488 | 1161 | 252 | 83 | | | |
| smorf513 | 489 | 1162 | 255 | 84 | 76 | 0.013 | gp:[GI:13359187] [LN:AB051444] [AC:AB051444] [PN:KIAA1657 |
| | | | | | | | protein] [GN:KIAA1657] [OR:Homo sapiens] [SR:Homo sapiens |
| | | | | | | | cDNA to mRNA, clone:hg00527] [DB:genpept-pri1] [DE:Homo |
| | | | | | | | sapiens mRNA for KIAA1657 protein, partial cds.] [NT:Start codon is |
| | | | | | | | not identified.] [LE:<6088] [RE:6471] [DI:direct] |
| smorf515 | 490 | 1163 | 222 | 73 | 71 | 0.043 | sp:[LN:YVAC_VACCC] [AC:P20512] [GN:A ORF C] [OR:Vaccinia |
| | | | | | | | virus] [SR:,strain Copenhagen] [DE: 14.4 KDA PROTEIN] |
| | | | | | | | [SP:P20512] [DB:swissprot]>pir:[LN:H42523] [AC:H42523] [PN:A- |
| | | | | | | | ORF-C protein] [OR:vaccinia virus] [DB:pir2]>gp:[GI:335473] |
| | | | | | | | [LN:VACCG] [AC:M35027] [OR:Vaccinia virus] [SR:Vaccinia virus |
| | | | | | | | (strain Copenhagen) DNA, clone VC-2] [DB:genpept-vrl2] |
| | | | | | | | [DE:Vaccinia virus, complete genome.] [NT:A ORF C; ] [LE:120025] |
| | | | | | | | [RE:120411] [DI:direct] |
| smorf516 | 491 | 1164 | 240 | 79 | 68 | 0.034 | pir:[LN:T44250] [AC:T44250] [PN:creatinase, [validated]] [GN:creA] |
| | | | | | | | [CL:X-Pro aminopeptidase] [OR:Arthrobacter sp.] [SR:strain TE1826, |
| | | | | | | | strain TE1826] [SR:strain TE1826, ] [EC:3.5.3.3] [DB:pir2]> |
| | | | | | | | gp:[GI:3116223] [LN:AB007122] [AC:AB007122] [PN:creatinase] |
| | | | | | | | [OR:Arthrobacter sp.] [SR:Arthrobacter sp. (strain:TE1826) DNA] |
| | | | | | | | [DB:genpept-bct1] [DE:Arthrobacter sp. gene for negative regulator, |
| | | | | | | | sarcosine oxidase, transporter, creatinase, creatininase and |
| | | | | | | | transporter, complete cds.] [LE:4061] [RE:5296] [DI:complement] |
| smorf517 | 492 | 1165 | 213 | 70 | 205 | 2.3E−15 | pir:[LN:T33894] [AC:T33894] [PN: protein Y37E11B.5] |
| | | | | | | | [GN:Y37E11B.5] [OR:Caenorhabditis elegans] [DB:pir2] [MP:4]> |
| | | | | | | | gp:[GI:4226107] [LN:CELY37E11B] [AC:AF125451] |
| | | | | | | | [GN:Y37E11B.5] [OR:Caenorhabditis elegans] [DB:genpept-inv3] |
| | | | | | | | [DE:Caenorhabditis elegans cosmid Y37E11B.] [NT:contains |
| | | | | | | | similarity to the NIFR3/SMM1 family; coded] |
| | | | | | | | [LE:16485:17403:18400] [RE:16779:17730:18682] [DI:complement |
| | | | | | | | Join] |
| smorf521 | 493 | 1166 | 393 | 130 | 74 | 0.037 | gp:[GI:12858110] [LN:AK018420] [AC:AK018420] [OR:Mus |
| | | | | | | | musculus] [SR:Mus musculus (strain:C57BL/6J) 16 days embryo |
| | | | | | | | lung cDNA to mRNA] [DB:genpept-htc] [DE:Mus musculus 16 days |
| | | | | | | | embryo lung cDNA, RIKEN full-length enrichedlibrary, |
| | | | | | | | clone:8430416G17, full insert sequence.] [NT:] [LE:184] [RE:495] |
| | | | | | | | [DI:direct] |
| smorf522 | 494 | 1167 | 234 | 77 | 90 | 0.0082 | pir:[LN:S74598] [AC:S74598] [PN: protein sll1040] |
| | | | | | | | [OR:Synechocystis sp.] [SR:PCC 6803, PCC 6803] [SR:PCC 6803, |
| | | | | | | | ] [DB:pir2]>gp:[GI:1651823] [LN:D90900] |
| | | | | | | | [AC:D90900:AB001339:BA000022] [GN:sll1040] [OR:Synechocystis |
| | | | | | | | sp. PCC 6803] [SR:Synechocystis sp. PCC 6803 (strain:PCC6803) |
| | | | | | | | DNA] [DB:genpept-bct3] [DE:Synechocystis sp. PCC 6803 DNA, |
| | | | | | | | complete genome, section:2/27,133860-271599.] |
| | | | | | | | [NT:ORF_ID:sll1040] [LE:52742] [RE:55039] [DI:complement] |
| smorf524 | 495 | 1168 | 192 | 63 | | | |
| smorf525 | 496 | 1169 | 156 | 51 | 54 | 0.032 | sp:[LN:Y489_RICPR] [AC:Q9ZD57] [GN:RP489] [OR:Rickettsia |
| | | | | | | | prowazekii] [DE: PROTEIN RP489] [SP:Q9ZD57] [DB:swissprot]> |
| | | | | | | | pir:[LN:D71652] [AC:D71652] [PN: protein RP489] [GN:RP489] |
| | | | | | | | [CL:Rickettsia prowazekii protein RP489] [OR:Rickettsia |
| | | | | | | | prowazekii] [DB:pir2]>gp:[GI:3861042] [LN:RPXX03] |
| | | | | | | | [AC:AJ235272:AJ235269] [PN: ] [GN:RP489] [OR:Rickettsia |
| | | | | | | | prowazekii] [DB:genpept-bct3] [DE:Rickettsia prowazekii strain |
| | | | | | | | Madrid E, complete genome; segment3/4.] [LE:8277] [RE:9143] |
| | | | | | | | [DI:complement] |
| smorf527 | 497 | 1170 | 174 | 57 | | | |
| smorf528 | 498 | 1171 | 291 | 96 | | | |
| smorf529 | 499 | 1172 | 240 | 79 | | | |
| smorf531 | 500 | 1173 | 405 | 134 | 84 | 0.016 | gp:[GI:10444169] [LN:AF288090] [AC:AF288090] |
| | | | | | | | [PN:succinate:cytochrome c oxidoreductase subunit 3] [GN:sdh3] |
| | | | | | | | [OR:Mitochondrion Rhodomonas salina] [SR:Rhodomonas salina] |
| | | | | | | | [DB:genpept-pln2] [EC:1.3.5.1] [DE:Rhodomonas salina |
| | | | | | | | mitochondrial DNA, complete genome.] [LE:16625] [RE:17011] |
| | | | | | | | [DI:complement] |
| smorf533 | 501 | 1174 | 201 | 66 | | | |
| smorf534 | 502 | 1175 | 201 | 66 | | | |
| smorf535 | 503 | 1176 | 204 | 67 | | | |
| smorf536 | 504 | 1177 | 222 | 73 | | | |
| smorf538 | 505 | 1178 | 210 | 69 | | | |
| smorf539 | 506 | 1179 | 177 | 58 | | | |
| smorf541 | 507 | 1180 | 144 | 47 | | | |
| smorf542 | 508 | 1181 | 261 | 86 | 85 | 0.012 | pir:[LN:T32516] [AC:T32516] [PN: protein C44B12.7] |
| | | | | | | | [GN:C44B12.7] [CL:Caenorhabditis elegans ZK1236.4 protein] |
| | | | | | | | [OR:Caenorhabditis elegans] [DB:pir2] [MP:4]>gp:[GI:2662564] |
| | | | | | | | [LN:AF036692] [AC:AF036692] [PN: protein C44B12.7] |
| | | | | | | | [GN:C44B12.7] [OR:Caenorhabditis elegans] [DB:genpept-inv2] |
| | | | | | | | [DE:Caenorhabditis elegans cosmid C44B12, complete sequence.] |
| | | | | | | | [LE:37086:38280] [RE:37889:38645] [DI:complement Join] |
| smorf545 | 509 | 1182 | 267 | 88 | 77 | 0.03 | gp:[GI:15025618] [LN:AE007757] [AC:AE007757:AE001437] |
| | | | | | | | [PN:Uncharacterized conserved membrane protein, YGGA] |
| | | | | | | | [GN:CAC2593] [OR:Clostridium acetobutylicum] [DB:genpept-bct1] |
| | | | | | | | [DE:Clostridium acetobutylicum ATCC824 section 245 of 356 of |
| | | | | | | | the complete genome.] [LE:428] [RE:1045] [DI:direct] |
| smorf547 | 510 | 1183 | 99 | 32 | | | |
| smorf548 | 511 | 1184 | 408 | 135 | | | |
| smorf549 | 512 | 1185 | 270 | 89 | 90 | 0.0027 | gp:[GI:14702103] [LN:AC006680] [AC:AC006680] [PN: protein |
| | | | | | | | R13D7.1] [GN:R13D7.1] [OR:Caenorhabditis elegans] [DB:genpept- |
| | | | | | | | inv1] [DE:Caenorhabditis elegans cosmid R13D7, complete |
| | | | | | | | sequence.] [LE:20325:20882] [RE:20824:21386] [DI:directJoin] |
| smorf550 | 513 | 1186 | 129 | 42 | | | |
| smorf552 | 514 | 1187 | 243 | 80 | 137 | 4.5E−09 | gp:[GI:2392026] [LN:SCU73805] [AC:U73805:U00091] |
| | | | | | | | [PN:Yal069wp] [GN:YAL069W] [OR:Saccharomyces cerevisiae] |
| | | | | | | | [SR:baker's yeast] [DB:genpept-pln4] [DE:Saccharomyces |
| | | | | | | | cerevisiae chromosome I left arm sequence.] [LE:335] [RE:649] |
| | | | | | | | [DI:direct] |
| smorf554 | 515 | 1188 | 183 | 60 | | | |
| smorf555 | 516 | 1189 | 192 | 63 | | | |
| smorf557 | 517 | 1190 | 132 | 43 | | | |
| smorf558 | 518 | 1191 | 204 | 67 | | | |
| smorf559 | 519 | 1192 | 267 | 88 | | | |
| smorf560 | 520 | 1193 | 111 | 36 | | | |
| smorf562 | 521 | 1194 | 261 | 86 | 84 | 0.012 | pir:[LN:T31826] [AC: T31826] [PN: protein C17E7.3] [GN:C17E7.3] |
| | | | | | | | [OR:Caenorhabditis elegans] [DB:pir2] [MP:5]>gp: [GI:2315381] |
| | | | | | | | [LN:AF016443] [AC: AF016443] [PN: protein C17E7.3] [GN:C17E7.3] |
| | | | | | | | [OR:Caenorhabditis elegans] [DB:genpept-inv2] [DE:Caenorhabditis |
| | | | | | | | elegans cosmid C17E7, complete sequence.] |
| | | | | | | | [LE:31970:32557:32766:33162] [RE:32117:32625:32918:33738] |
| | | | | | | | [DI:direct Join] |
| smorf563 | 522 | 1195 | 207 | 68 | | | |
| smorf567 | 523 | 1196 | 246 | 81 | 49 | 0.048 | pir:[LN:T07315] [AC:T07315] [PN:protein 46c] [OR:chloroplast |
| | | | | | | | Chlorella vulgaris] [DB:pir2]>gp:[GI: 2224479] [LN:AB001684] |
| | | | | | | | [AC:AB001684] [OR:Chloroplast Chlorella vulgaris] [SR:Chlorella |
| | | | | | | | vulgaris chloroplast DNA] [DB:genpept-pln 1] [DE:Chlorella vulgaris C |
| | | | | | | | 27 chloroplast DNA, complete sequence.] [NT:ORF46c] [LE:107657] |
| | | | | | | | [RE:107797] [DI:complement] |
| smorf568 | 524 | 1197 | 237 | 78 | | | |
| smorf569 | 525 | 1198 | 195 | 64 | | | |
| smorf571 | 526 | 1199 | 303 | 100 | | | |
| smorf573 | 527 | 1200 | 315 | 104 | | | |
| smorf574 | 528 | 1201 | 249 | 82 | 81 | 0.017 | pir:[LN:A60944] [AC:A60944] [PN:ubiquinol-cytochrome-c |
| | | | | | | | reductase, cytochrome b] [CL:cytochrome b:cytochrome b |
| | | | | | | | homology:cytochrome b6 homology:plastoquinol-plastocyanin |
| | | | | | | | reductase 17K protein homology] [OR:mitochondrion Leishmania |
| | | | | | | | mexicana amazonensis] [EC:1.10.2.2] [DB:pir2] |
| smorf575 | 529 | 1202 | 279 | 92 | 235 | 1.8E−19 | pir:[LN:S70302] [AC:S70302] [PN: protein YBL109w] [GN:YBL109w] |
| | | | | | | | [OR:Saccharomyces cerevisiae] [DB:pir2] [MP:2L] |
| smorf576 | 530 | 1203 | 234 | 77 | 112 | 0.000002 | sp:[LN:YFG3_YEAST] [AC:P43541] [GN:YFL063W] |
| | | | | | | | [OR:Saccharomyces cerevisiae] [SR:,Baker's yeast] [DE: 17.5 KDA |
| | | | | | | | PROTEIN IN THI5 5′REGION] [SP:P43541] [DB:swissprot]> |
| | | | | | | | pir:[LN:S56192] [AC:S56192:S62274] [PN: membrane protein |
| | | | | | | | YFL063w: protein F008] [OR: Saccharomyces cerevisiae] [DB:pir2] |
| | | | | | | | [MP:6L]>gp:[GI:836692] [LN:YSCCHRVIN] [AC:D50617: D31600: |
| | | | | | | | D44594: D44595: D44596: D44597: D44598: D44599: D44600] |
| | | | | | | | [OR:Saccharomyces cerevisiae] [SR:Saccharomyces cerevisiae |
| | | | | | | | (strain:AB972) DNA] [DB:genpept-pln4] [DE:Saccharomyces |
| | | | | | | | cerevisiae chromosome VI complete DNA sequence.] |
| | | | | | | | [NT:YFL063W] [LE:5066] [RE:5521] [DI:direct] |
| smorf578 | 531 | 1204 | 123 | 40 | | | |
| smorf581 | 532 | 1205 | 222 | 73 | | | |
| smorf582 | 533 | 1206 | 252 | 83 | | | |
| smorf583 | 534 | 1207 | 201 | 66 | | | |
| smorf584 | 535 | 1208 | 129 | 42 | | | |
| smorf585 | 536 | 1209 | 180 | 59 | 76 | 0.017 | pir:[LN:S43955] [AC:S43955] [PN:NADH dehydrogenase |
| | | | | | | | (ubiquinone), chain 3, kinetoplast:CR5 protein:NADH:ubiquinone |
| | | | | | | | oxidoreductase] [GN:nd3] [OR:mitochondrion Trypanosoma brucei] |
| | | | | | | | [EC:1.6.5.3] [DB:pir2] |
| smorf586 | 537 | 1210 | 300 | 99 | 152 | 1.1E−10 | gp:[GI:12718388] [LN:NCB11N2] [AC:AL513444] [PN:conserved |
| | | | | | | | protein] [GN:B11N2.150] [OR:Neurospora crassa] [DB:genpept-pln3] |
| | | | | | | | [DE:Neurospora crassa DNA linkage group V BAC contig B11N2.] |
| | | | | | | | [NT:similarity to clone:k3k7, chromosome 5, arabidopsis] |
| | | | | | | | [LE:48041:48132:48313] [RE:48073:48258:48494] [DI:directJoin] |
| smorf589 | 538 | 1211 | 216 | 71 | 55 | 0.021 | gp:[GI:12721132] [LN:AE006121] [AC:AE006121:AE004439] [PN:] |
| | | | | | | | [GN:PM0825] [OR:Pasteurella multocida] [DB:genpept-bct1] |
| | | | | | | | [DE:Pasteurella multocida PM70 section 88 of 204 of the complete |
| | | | | | | | genome.] [LE:4079] [RE:4618] [DI:complement] |
| smorf592 | 539 | 1212 | 141 | 46 | | | |
| smorf593 | 540 | 1213 | 222 | 73 | | | |
| smorf594 | 541 | 1214 | 138 | 45 | | | |
| smorf596 | 542 | 1215 | 99 | 32 | | | |
| smorf597 | 543 | 1216 | 273 | 90 | 232 | 3.9E−18 | sp:[LN:TOP3_YEAST] [AC:P13099] |
| | | | | | | | [GN:TOP3:EDR1:YLR234W:L8083.3] [OR:Saccharomyces |
| | | | | | | | cerevisiae] [SR:Baker's yeast] [EC:5.99.1.2] [DE:DNA |
| | | | | | | | TOPOISOMERASE III,] [SP:P13099] [DB:swissprot]> |
| | | | | | | | pir:[LN:ISBYT3] [AC:A33169:S51455] |
| smorf599 | 544 | 1217 | 231 | 76 | 136 | 0.00000008 | sp:[LN:TOP3_YEAST] [AC:P13099] |
| | | | | | | | [GN:TOP3:EDR1:YLR234W:L8083.3] [OR:Saccharomyces |
| | | | | | | | cerevisiae] [SR:,Baker's yeast] [EC:5.99.1.2] [DE:DNA |
| | | | | | | | TOPOISOMERASE III,] [SP:P13099] [DB:swissprot]> |
| | | | | | | | pir:[LN:ISBYT3] [AC:A33169:S51455] |
| smorf602 | 545 | 1218 | 114 | 37 | | | |
| smorf603 | 546 | 1219 | 183 | 60 | | | |
| smorf606 | 547 | 1220 | 135 | 44 | 74 | 0.029 | gp:[GI:15130933] [LN:SEN320483] [AC:AJ320483] [PN:SciR protein] |
| | | | | | | | [GN:sciR] [FN: periplasmic protein] [OR:Salmonella enterica subsp. |
| | | | | | | | enterica serovar Typhimurium] [DB:genpept-bct3] [DE:Salmonella |
| | | | | | | | enterica subsp. enterica serovar Typhimurium DNA forcentisome 7 |
| | | | | | | | genomic island.] [LE:19028] [RE:19471] [DI:direct] |
| smorf607 | 548 | 1221 | 222 | 73 | | | |
| smorf608 | 549 | 1222 | 186 | 61 | | | |
| smorf609 | 550 | 1223 | 222 | 73 | | | |
| smorf610 | 551 | 1224 | 162 | 53 | | | |
| smorf611 | 552 | 1225 | 165 | 54 | | | |
| smorf612 | 553 | 1226 | 108 | 35 | | | |
| smorf613 | 554 | 1227 | 78 | 25 | | | |
| smorf614 | 555 | 1228 | 189 | 62 | | | |
| smorf615 | 556 | 1229 | 198 | 65 | 73 | 0.027 | pir:[LN:T28395] [AC:T28395] [PN:ORF MSV233 protein] |
| | | | | | | | [OR:Melanoplus sanguinipes entomopoxvirus] [DB:pir2]> |
| | | | | | | | gp:[GI:4049785] [LN:AF063866] [AC:AF063866] [PN:ORF MSV233 |
| | | | | | | | protein] [GN:MSV233] [OR:Melanoplus sanguinipes entomopoxvirus] |
| | | | | | | | [DB:genpept-vrl1] [DE:Melanoplus sanguinipes entomopoxvirus, |
| | | | | | | | complete genome.] [LE:201518] [RE:201796] [DI:complement] |
| smorf616 | 557 | 1230 | 123 | 40 | | | |
| smorf618 | 558 | 1231 | 159 | 52 | | | |
| smorf619 | 559 | 1232 | 246 | 81 | | | |
| smorf620 | 560 | 1233 | 180 | 59 | | | |
| smorf622 | 561 | 1234 | 156 | 51 | | | |
| smorf623 | 562 | 1235 | 249 | 82 | | | |
| smorf624 | 563 | 1236 | 249 | 82 | | | |
| smorf627 | 564 | 1237 | 237 | 78 | 78 | 0.022 | gp:[GI:10176977] [LN:AB010077] [AC:AB010077:BA000015] |
| | | | | | | | [PN:40S ribosomal protein S9] [OR:Arabidopsis thaliana] |
| | | | | | | | [SR:Arabidopsis thaliana (strain:Columbia) DNA, clone_lib:Mitsui P] |
| | | | | | | | [DB:genpept-pln1] [DE:Arabidopsis thaliana genomic DNA, |
| | | | | | | | chromosome 5, P1 clone:MYH19.] [NT:gene_id:MYH19.1] |
| | | | | | | | [LE:2637:2991:3572] [RE:2664:3372:3755] [DI:directJoin] |
| smorf629 | 565 | 1238 | 201 | 66 | | | |
| smorf630 | 566 | 1239 | 243 | 80 | 94 | 0.00016 | pir:[LN:S51339] [AC:S51339] [PN: membrane protein YLR334c: |
| | | | | | | | protein L8300.11] [GN:YLR334c] [OR:Saccharomyces cerevisiae] |
| | | | | | | | [DB:pir2] [MP:12R]>gp:[GI:609390] [LN:YSCL8300] |
| | | | | | | | [AC:U19028:Y13138] [PN:Ylr334cp] [GN:YLR334C] |
| | | | | | | | [OR:Saccharomyces cerevisiae] [SR:baker's yeast strain=S288C |
| | | | | | | | (AB972)] [DB:genpept-pln4] [DE:Saccharomyces cerevisiae |
| | | | | | | | chromosome XII cosmid 8300.] [LE:4182] [RE:4562] |
| | | | | | | | [DI:complement] |
| smorf633 | 567 | 1240 | 234 | 77 | 213 | 3.9E−17 | pir:[LN:S70302] [AC:S70302] [PN: protein YBL109w] [GN:YBL109w] |
| | | | | | | | [OR:Saccharomyces cerevisiae] [DB:pir2] [MP:2L] |
| smorf634 | 568 | 1241 | 234 | 77 | 125 | 8.3E−08 | pir:[LN:S70302] [AC:S70302] [PN: protein YBL109w] [GN:YBL109w] |
| | | | | | | | [OR:Saccharomyces cerevisiae] [DB:pir2] [MP:2L] |
| smorf636 | 569 | 1242 | 186 | 61 | | | |
| smorf637 | 570 | 1243 | 186 | 61 | | | |
| smorf638 | 571 | 1244 | 297 | 98 | | | |
| smorf639 | 572 | 1245 | 216 | 71 | | | |
| smorf642 | 573 | 1246 | 207 | 68 | | | |
| smorf645 | 574 | 1247 | 240 | 79 | | | |
| smorf646 | 575 | 1248 | 183 | 60 | | | |
| smorf647 | 576 | 1249 | 78 | 25 | | | |
| smorf648 | 577 | 1250 | 162 | 53 | | | |
| smorf649 | 578 | 1251 | 108 | 35 | | | |
| smorf650 | 579 | 1252 | 198 | 65 | | | |
| smorf651 | 580 | 1253 | 198 | 65 | 73 | 0.027 | gp:[GI:10178678] [LN:AF295546] [AC:AF295546] [PN:orf120] |
| | | | | | | | [GN:orf120] [OR:Mitochondrion Malawimonas jakobiformis] |
| | | | | | | | [SR:Malawimonas jakobiformis] [DB:genpept-inv3] |
| | | | | | | | [DE:Malawimonas jakobiformis mitochondrial DNA, complete |
| | | | | | | | genome.] [LE:12057] [RE:12419] [DI:complement] |
| smorf652 | 581 | 1254 | 171 | 56 | | | |
| smorf654 | 582 | 1255 | 180 | 59 | 77 | 0.047 | pir:[LN:S59078] [AC:S59078] [PN:conserved protein 262] |
| | | | | | | | [CL:conserved protein HI0188] [OR:mitochondrion Chondrus crispus] |
| | | | | | | | [SR: carragheen] [DB:pir2] |
| smorf656 | 583 | 1256 | 180 | 59 | | | |
| smorf657 | 584 | 1257 | 255 | 84 | 63 | 0.0085 | pir:[LN:T29273] [AC:T29273] [PN: protein T01C4.4] [GN:T01C4.4] |
| | | | | | | | [OR:Caenorhabditis elegans] [DB:pir2] [MP:5]>gp:[GI:1572838] |
| | | | | | | | [LN:U70858] [AC:U70858] [PN: protein T01C4.4] [GN:T01C4.4] |
| | | | | | | | [OR: Caenorhabditis elegans] [DB:genpept-inv4] [DE:Caenorhabditis |
| | | | | | | | elegans cosmid T01C4, complete sequence.] [NT:weak similarity to |
| | | | | | | | family 1 of G-protein coupled] [LE:15768:16134:16238] |
| | | | | | | | [RE:15995:16193:16615] [DI:complementJoin] |
| smorf658 | 585 | 1258 | 207 | 68 | | | |
| smorf659 | 586 | 1259 | 258 | 85 | 73 | 0.027 | gp:[GI:11545456] [LN:AF298190] [AC:AF298190] [PN:] |
| | | | | | | | [OR:Sinorhizobium meliloti] [DB:genpept-bct2] [DE:Sinorhizobium |
| | | | | | | | meliloti transposase Tnp149 (tnp149) gene,partial cds; methyl- |
| | | | | | | | accepting-chemotaxis-protein (mcpY) gene,complete cds; and NAD- |
| | | | | | | | dependent formate dehydrogenase operon,partial sequence.] |
| | | | | | | | [NT:0rf86] [LE:6678] [RE:6938] [DI:complement] |
| smorf661 | 587 | 1260 | 186 | 61 | | | |
| smorf662 | 588 | 1261 | 165 | 54 | | | |
| smorf663 | 589 | 1262 | 285 | 94 | | | |
| smorf665 | 590 | 1263 | 225 | 74 | | | |
| smorf666 | 591 | 1264 | 252 | 83 | | | |
| smorf673 | 592 | 1265 | 153 | 50 | | | |
| smorf010 | 593 | 1266 | 1320 | 440 | 2212 | 5.8E−229 | pir:[LN:S47536] [AC:S47536:S53461:S53463:S43081] [PN:SWH1 |
| | | | | | | | protein:protein YAR042w:protein YAR044w] [GN:SWH1:OSH1] |
| | | | | | | | [CL:unassigned ankyrin repeat proteins:ankyrin repeat |
| | | | | | | | homology:EGF homology] [OR:Saccharomyces cerevisiae] [DB:pir2] |
| | | | | | | | [MP:1R]>gp:[GI:402658] [LN:SCSWH1] [AC:X74552] [GN:SWH1] |
| | | | | | | | [OR:Saccharomyces cerevisiae] [SR:baker's yeast] [DB:genpept- |
| | | | | | | | pln4] [DE:S.cerevisiae SWH1 gene.] [SP:P39555] [LE:369] |
| | | | | | | | [RE:3941] [DI:direct] |
| smorf030 | 594 | 1267 | 156 | 51 | 266 | 8.8E−22 | gp:[GI:3152696] [LN:AF065148] [AC:AF065148] [PN: very long- |
| | | | | | | | chain fatty acyl-CoA synthetase] [GN:FAT1] [OR:Saccharomyces |
| | | | | | | | cerevisiae] [SR:baker's yeast] [DB:genpept-pln1] |
| | | | | | | | [DE:Saccharomyces cerevisiae very long-chain fatty acyl-CoA |
| | | | | | | | synthetase(FAT1) gene, complete cds.] [NT:Fat1p] [LE:197] |
| | | | | | | | [RE:2206] [DI:direct] |
| smorf035 | 595 | 1268 | 78 | 25 | 93 | 0.0028 | sp:[LN:SYKC_YEAST] [AC:P15180] |
| | | | | | | | [GN:KRS1:GCD5:YDR037W:YD9673.09] [OR:Saccharomyces |
| | | | | | | | cerevisiae] [SR:Baker's yeast] [EC:6.1.1.6] [DE:(LYSRS)] |
| | | | | | | | [SP:P15180] [DB:swissprot] |
| smorf037 | 596 | 1269 | 102 | 33 | 151 | 1.7E−09 | sp:[LN:SYKC_YEAST] [AC:P15180] |
| | | | | | | | [GN:KRS1:GCD5:YDR037W:YD9673.09] [OR:Saccharomyces |
| | | | | | | | cerevisiae] [SR:,Baker's yeast] [EC:6.1.1.6] [DE:(LYSRS)] |
| | | | | | | | [SP:P15180] [DB:swissprot] |
| smorf040 | 597 | 1270 | 216 | 71 | 163 | 8.7E−11 | sp:[LN:SYKC_YEAST] [AC:P15180] |
| | | | | | | | [GN:KRS1:GCD5:YDR037W:YD9673.09] [OR:Saccharomyces |
| | | | | | | | cerevisiae] [SR:,Baker's yeast] [EC:6.1.1.6] [DE:(LYSRS)] |
| | | | | | | | [SP:P15180] [DB:swissprot] |
| smorf061 | 598 | 1271 | 282 | 93 | 498 | 2.5E−47 | sp:[LN:YJ9Z_YEAST] [AC:P47188] [GN:YJR162C:J2420] |
| | | | | | | | [OR:Saccharomyces cerevisiae] [SR:,Baker's yeast] [DE: 13.4 KDA |
| | | | | | | | PROTEIN IN SOR1 3′REGION] [SP:P47188] [DB:swissprot]> |
| | | | | | | | pir:[LN:S57192] [AC:S57192] [PN: protein YKL225w homolog |
| | | | | | | | YJR162c: protein J2420: protein YJR162c] [GN:YJR162c] |
| | | | | | | | [OR:Saccharomyces cerevisiae] [DB:pir2] [MP:10R]> |
| | | | | | | | gp:[GI:1015925] [LN:SCYJR162C] [AC:Z49662:Y13136] |
| | | | | | | | [OR:Saccharomyces cerevisiae] [SR:baker's yeast] [DB:genpept- |
| | | | | | | | pln4] [DE:S.cerevisiae chromosome X reading frame ORF |
| | | | | | | | YJR162c.] [NT:ORF YJR162c] [SP:P47188] [LE:912] [RE:1262] |
| | | | | | | | [DI:complement] |
| smorf063 | 599 | 1272 | 1113 | 370 | 1800 | 2.7E−185 | pir:[LN:T29093] [AC:T29093] [PN: protein] [OR:Saccharomyces |
| | | | | | | | paradoxus] [DB:pir2]>gp:[GI:2865202] [LN:SPU19263] [AC:U19263] |
| | | | | | | | [OR:Saccharomyces paradoxus] [DB:genpept-pln4] |
| | | | | | | | [DE:Saccharomyces paradoxus retrotransposon Ty5-6p associated |
| | | | | | | | with autonomously replicating sequence, complete sequence.] |
| | | | | | | | [NT:ORF] [LE:1441] [RE:6321] [DI:direct] |
| smorf064 | 600 | 1273 | 291 | 96 | 455 | 2.7E−41 | pir:[LN:T29093] [AC:T29093] [PN: protein] [OR:Saccharomyces |
| | | | | | | | paradoxus] [DB:pir2]>gp:[GI:2865202] [LN:SPU19263] [AC:U19263] |
| | | | | | | | [OR:Saccharomyces paradoxus] [DB:genpept-pln4] |
| | | | | | | | [DE:Saccharomyces paradoxus retrotransposon Ty5-6p associated |
| | | | | | | | withautonomously replicating sequence, complete sequence.] |
| | | | | | | | [NT:ORF] [LE:1441] [RE:6321] [DI:direct] |
| smorf065 | 601 | 1274 | 1242 | 414 | 2065 | 2.2E−213 | sp:[LN:YK85_YEAST] [AC:P36172] [GN:YKR105C] |
| | | | | | | | [OR:Saccharomyces cerevisiae] [SR:,Baker's yeast] [DE: 63.4 KDA |
| | | | | | | | PROTEIN IN SIR1 3'REGION] [SP:P36172] [DB:swissprot]> |
| | | | | | | | pir:[LN:S38184] [AC:S38184] [PN: protein YCL069W homolog |
| | | | | | | | YKR105c] [CL:conserved protein YCL069w] [OR:Saccharomyces |
| | | | | | | | cerevisiae] [DB:pir2] [MP:11R]>gp:[GI:486615] [LN:SCYKR105C] |
| | | | | | | | [AC:Z28330:Y13137] [OR:Saccharomyces cerevisiae] [SR:baker's |
| | | | | | | | yeast] [DB:genpept-pln4] [DE:S.cerevisiae chromosome XI reading |
| | | | | | | | frame ORF YKR105c.] [NT:ORF YKR105c] [SP:P36172] [LE:960] |
| | | | | | | | [RE:2708] [DI:complement] |
| smorf067 | 602 | 1275 | 1242 | 413 | 1917 | 1.1E−197 | gp:[GI:14588900] [LN:SCCHRIII] |
| | | | | | | | [AC:X59720:S43845:S49180:S58084:S93798] [PN: protein] |
| | | | | | | | [OR:Saccharomyces cerevisiae] [SR:baker's yeast] [DB:genpept- |
| | | | | | | | pln4] [DE:S.cerevisiae chromosome III complete DNA sequence.] |
| | | | | | | | [NT:ORF YCL061c] [LE:18816] [RE:22106] [DI:complement] |
| smorf075 | 603 | 1276 | 336 | 111 | 583 | 2.4E−56 | sp:[LN:YCB0_YEAST] [AC:P25554:P87008] |
| | | | | | | | [GN:YCL010C:YCL10C] [OR:Saccharomyces cerevisiae] |
| | | | | | | | [SR:,Baker's yeast] [DE: 29.4 KDA PROTEIN IN GBP2-ILV6 |
| | | | | | | | INTERGENIC REGION] [SP:P25554:P87008] [DB:swissprot]> |
| | | | | | | | pir:[LN:S74287] [AC:S74287:S19337] [PN: protein YCL010c] |
| | | | | | | | [OR:Saccharomyces cerevisiae] [DB:pir2] [MP:3L]>gp:[GI:1907134] |
| | | | | | | | [LN:SCCHRIII] [AC:X59720: S43845: S49180: S58084: S93798] |
| | | | | | | | [PN: protein] [OR:Saccharomyces cerevisiae] [SR:baker's yeast] |
| | | | | | | | [DB:genpept-pln4] [DE:S.cerevisiae chromosome III complete DNA |
| | | | | | | | sequence.] [NT:ORF YCL010c -strong similarity to Saccharomyces] |
| | | | | | | | [SP:P25554] [LE:103566] [RE:104345] [DI:complement] |
| smorf076 | 604 | 1277 | 279 | 92 | 468 | 3.8E−44 | gp:[GI:2252812] [LN:AF004731] [AC:AF004731] [PN:Stp22p] |
| | | | | | | | [GN:STP22] [FN:required for vacuolar targeting of] |
| | | | | | | | [OR:Saccharomyces cerevisiae] [SR:baker's yeast] [DB:genpept- |
| | | | | | | | pln1] [DE:Saccharomyces cerevisiae Stp22p (STP22) gene, |
| | | | | | | | complete cds.] [NT:similar to the mouse and human Tsg101 tumor] |
| | | | | | | | [LE:383] [RE:1540] [DI:direct] |
| smorf077 | 605 | 1278 | 249 | 82 | 293 | 4.8E−25 | pir:[LN:T11166] [AC:T11166: S74289: S59798: S19379: S19368: |
| | | | | | | | S60383: S59422] [PN:CDPdiacylglycerol-serine O- |
| | | | | | | | phosphatidyltransferase, PGS1:phosphatidylserine synthase:protein |
| | | | | | | | YCL003w:protein YCL004w] [GN:PGS1: PEL1: YCL003w: |
| | | | | | | | YCL004w] [OR:Saccharomyces cerevisiae] [EC:2.7.8.8] [DB:pir2] |
| | | | | | | | [MP:3L]>gp:[GI:14588923] [LN:SCCHRIII] [AC:X59720: S43845: |
| | | | | | | | S49180: S58084: S93798] [PN:phosphatidyl glycerophosphate |
| | | | | | | | synthase] [GN:PGS1] [OR:Saccharomyces cerevisiae] [SR:baker's |
| | | | | | | | yeast] [DB:genpept-pln4] [DE:S.cerevisiae chromosome III complete |
| | | | | | | | DNA sequence.] [NT:ORF YCL004w] [LE:109101] [RE:110666] |
| | | | | | | | [DI:direct]>gp:[GI:3808176] [LN:SCE012047] [AC:AJ012047] |
| | | | | | | | [PN:phosphatidyl glycerophosphate synthase] [GN:PSG1] |
| | | | | | | | [OR:Saccharomyces cerevisiae] [SR:baker's yeast] [DB:genpept- |
| | | | | | | | pln4] [DE:Saccharomyces cerevisiae PGS1 gene.] [LE:1] [RE:1566] |
| | | | | | | | [DI:direct] |
| smorf078 | 606 | 1279 | 723 | 240 | 1159 | 2.2E−117 | sp:[LN:PEL1_YEAST] [AC:P25578:P25570:P87011] |
| | | | | | | | [GN:PEL1:YCL004W:YCL4W/3W] [OR:Saccharomyces cerevisiae] |
| | | | | | | | [SR:Baker's yeast] [EC:2.7.8.8] [DE:(EC 2.7.8.8) |
| | | | | | | | (PHOSPHATIDYLSERINE SYNTHASE)] |
| | | | | | | | [SP:P25578:P25570:P87011] [DB:swissprot] |
| smorf084 | 607 | 1280 | 768 | 255 | 1111 | 2.7E−112 | sp:[LN:YCS0_YEAST] [AC:P25623:P25622] |
| | | | | | | | [GN:YCR030C:YCR30C/YCR29C] [OR:Saccharomyces cerevisiae] |
| | | | | | | | [SR:,Baker's yeast] [DE: 96.1 KDA PROTEIN IN RIM1-RPS14A |
| | | | | | | | INTERGENIC REGION] [SP:P25623:P25622] [DB:swissprot]> |
| | | | | | | | pir:[LN:S74291] [AC:S74291:S40970:S19442:S19440] [PN: protein |
| | | | | | | | YCR030c: protein YCR029c] [OR:Saccharomyces cerevisiae] |
| | | | | | | | [DB:pir2] [MP:3R] |
| smorf085 | 608 | 1281 | 363 | 120 | 446 | 7.7E−41 | sp:[LN:PWP2_YEAST] [AC:P25635:P25633:P25636] |
| | | | | | | | [GN:PWP2:YCR055C:YCR55C/57C/58C] [OR:Saccharomyces |
| | | | | | | | cerevisiae] [SR:,Baker's yeast] [DE:PERIODIC TRYPTOPHAN |
| | | | | | | | PROTEIN 2] [SP:P25635:P25633:P25636] [DB:swissprot]> |
| | | | | | | | pir:[LN:S44226] [AC:S44226:S19469:S19471:S19472:S7 |
| smorf088 | 609 | 1282 | 273 | 90 | 403 | 2.9E−37 | pir:[LN:S74292] [AC:S74292] [PN: protein YCR068w-a] |
| | | | | | | | [GN:YCR068w-a] [CL:Saccharomyces protein YCR068w-a] |
| | | | | | | | [OR:Saccharomyces cerevisiae] [DB:pir2] [MP:3R] |
| smorf092 | 610 | 1283 | 246 | 81 | 294 | 1E−25 | sp:[LN:YH17_YEAST] [AC:P38898] [GN:YHR217C] |
| | | | | | | | [OR:Saccharomyces cerevisiae] [SR:,Baker's yeast] [DE: 17.1 KDA |
| | | | | | | | PROTEIN IN PUR5 3′REGION] [SP:P38898] [DB:swissprot]> |
| | | | | | | | pir:[LN:S48998] [AC:S48998] [PN: protein YHR217c] |
| | | | | | | | [GN:YHR217c] [OR:Saccharomyces cerevisiae] [DB:pir2] [MP:8R]> |
| | | | | | | | gp:[GI:551324] [LN:YSCH9177] [AC:U00029:U00093] |
| | | | | | | | [PN:Yhr217cp] [GN:YHR217c] [OR:Saccharomyces cerevisiae] |
| | | | | | | | [SR:baker's yeast strain=S288C (AB972)] [DB:genpept-pln4] |
| | | | | | | | [DE:Saccharomyces cerevisiae chromosome VIII cosmid 9177.] |
| | | | | | | | [LE:50035] [RE:50496] [DI:complement] |
| smorf096 | 611 | 1284 | 243 | 80 | 369 | 1.2E−33 | sp:[LN:YEI3_YEAST] [AC:P39974] [GN:YEL073C] |
| | | | | | | | [OR:Saccharomyces cerevisiae] [SR:,Baker's yeast] [DE: 12.0 KDA |
| | | | | | | | PROTEIN IN HXT8 5′REGION] [SP:P39974] [DB:swissprot]> |
| | | | | | | | pir:[LN:S50516] [AC:S50516] [PN: protein YEL073c] |
| | | | | | | | [GN:YEL073c] [OR:Saccharomyces cerevisiae] [DB:pir2] [MP:5L]> |
| | | | | | | | gp:[GI:603245] [LN:SCE9669] [AC:U18795:U00092] [PN:YeI073cp] |
| | | | | | | | [GN:YEL073C] [OR:Saccharomyces cerevisiae] [SR:baker's yeast |
| | | | | | | | strain=S288C (AB972)] [DB:genpept-pln4] [DE:Saccharomyces |
| | | | | | | | cerevisiae chromosome V cosmids 9669, 8334, 8199, and lambda |
| | | | | | | | clone 1160.] [LE:4753] [RE:5076] [DI:complement] |
| smorf097 | 612 | 1285 | 1251 | 416 | 1846 | 3.6E−190 | sp:[LN:AADE_YEAST] [AC:P42884] [GN:AAD14:YNL331C:N0300] |
| | | | | | | | [OR:Saccharomyces cerevisiae] [SR:Baker's yeast] [EC:1.1.1.-] [DE: |
| | | | | | | | ARYL-ALCOHOL DEHYDROGENASE AAD14] [SP:P42884] |
| | | | | | | | [DB:swissprot]>pir:[LN:S51335] |
| | | | | | | | [AC:S51335:S57392:S63314:S63317] |
| smorf107 | 613 | 1286 | 4044 | 1347 | 7060 | 0 | gp:[GI:836753] [LN:YSCCHRVIN] [AC:D50617: D31600: D44594: |
| | | | | | | | D44595: D44596: D44597: D44598: D44599: D44600] |
| | | | | | | | [PN:transposon TY1-17 154.0KD protein] [GN:TyB] |
| | | | | | | | [OR:Saccharomyces cerevisiae] [SR:Saccharomyces cerevisiae |
| | | | | | | | (strain:AB972) DNA] [DB:genpept-pln4] [DE:Saccharomyces |
| | | | | | | | cerevisiae chromosome VI complete DNA sequence.] [NT:Ty |
| | | | | | | | element] [LE:139471] [RE:143511] [DI:direct] |
| smorf111 | 614 | 1287 | 3987 | 1328 | 6917 | 0 | pir:[LN:S69979] [AC:S69979] [PN:TyB protein:protein P0729] |
| | | | | | | | [CL:TyB protein] [OR:Saccharomyces cerevisiae] [DB:pir2] [MP:16L]> |
| | | | | | | | gp:[GI:1370529] [LN:SCYPL257W] [AC:Z73613:U00094] |
| | | | | | | | [GN:TY1B] [OR:Saccharomyces cerevisiae] [SR:baker's yeast] |
| | | | | | | | [DB:genpept-pln4] [DE:S.cerevisiae chromosome XVI reading frame |
| | | | | | | | ORF YPL257w.] [LE:1595:2901] [RE:2899:6863] [DI:directJoin]> |
| | | | | | | | gp:[GI:1370534] [LN:SCYPL258C] [AC:Z73614:U00094] |
| | | | | | | | [GN:TY1B] [OR:Saccharomyces cerevisiae] [SR:baker's yeast] |
| | | | | | | | [DB:genpept-pln4] [DE:S.cerevisiae chromosome XVI reading frame |
| | | | | | | | ORF YPL258c.] [LE:4077:5383] [RE:5381:9345] [DI:direct Join] |
| smorf113 | 615 | 1288 | 1335 | 444 | 2336 | 4.2E−242 | pir:[LN:S40909] [AC:S40909:S69981] [PN:TyA protein:protein |
| | | | | | | | P9659_6_d:protein YAR010c] [CL:TyA protein] [OR:Saccharomyces |
| | | | | | | | cerevisiae] [DB:pir2] [MP:16R]>gp:[GI:2564963] [LN:YSCCHROMI] |
| | | | | | | | [AC:L22015:U00091] [PN:Yar010cp] [GN:YAR010C] |
| | | | | | | | [OR:Saccharomyces cerevisiae] [SR:baker's yeast] [DB:genpept- |
| | | | | | | | pln4] [DE:Saccharomyces cerevisiae chromosome I centromere and |
| | | | | | | | right armsequence.] [LE:30989] [RE:32311] [DI:complement] |
| smorf119 | 616 | 1289 | 1212 | 403 | 2133 | 1.4E−220 | gp:[GI:1289285] [LN:SC9395] [AC:Z46757:Z71256] [PN: ] |
| | | | | | | | [GN:truncated TYB] [OR:Saccharomyces cerevisiae] [SR:baker's |
| | | | | | | | yeast] [DB:genpept-pln4] [DE:S.cerevisiae chromosome IV cosmid |
| | | | | | | | 9395.] [NT:Protein sequence is in conflict with the conceptual] |
| | | | | | | | [LE:3882] [RE:5093] [DI:direct] |
| smorf120 | 617 | 1290 | 1497 | 498 | 2622 | 2.1E−272 | gp:[GI:1289295] [LN:SC9395] [AC:Z46727:Z71256] [PN: ] |
| | | | | | | | [OR:Saccharomyces cerevisiae] [SR:baker's yeast] [DB:genpept- |
| | | | | | | | pln4] [DE:S.cerevisiae chromosome IV cosmid 9395.] [NT:Protein |
| | | | | | | | sequence is in conflict with the conceptual] [LE:18732] [RE:20228] |
| | | | | | | | [DI:direct] |
| smorf124 | 618 | 1291 | 4044 | 1347 | 7067 | 0 | gp:[GI:1122340] [LN:SC8142A] [AC:Z68194:Z71256] [PN: ] [GN:TyB] |
| | | | | | | | [OR:Saccharomyces cerevisiae] [SR:baker's yeast] [DB:genpept- |
| | | | | | | | pln4] [DE:S.cerevisiae chromosome IV cosmid 8142A.] [NT:Protein |
| | | | | | | | sequence is in conflict with the conceptual] [LE:15257] [RE:19300] |
| | | | | | | | [DI:direct] |
| smorf125 | 619 | 1292 | 324 | 107 | 473 | 1.1E−44 | gp:[GI:496672] [LN:SCDNCH2] [AC:X79489] [PN:D-104 protein] |
| | | | | | | | [GN:YBL0822a] [OR:Saccharomyces cerevisiae] [SR:baker's yeast] |
| | | | | | | | [DB:genpept-pln4] [DE:S.cerevisiae genomic DNA, chromosome II |
| | | | | | | | from Y element to ILS1gene.] [LE:27160] [RE:27474] |
| | | | | | | | [DI:complement] |
| smorf126 | 620 | 1293 | 3987 | 1328 | 6936 | 0 | sp:[LN:YMD9_YEAST] [AC:Q03434] [GN:TY1B: YML039W: |
| | | | | | | | YM8054.04] [OR:Saccharomyces cerevisiae] [SR:,Baker's yeast] |
| | | | | | | | [DE:TRANSPOSON TY1 PROTEIN B] [SP:Q03434] [DB:swissprot]> |
| | | | | | | | pir:[LN:S52481] [AC:S52481] [PN:TyB protein:protein YML039w] |
| | | | | | | | [CL:TyB protein] [OR:Saccharomyces cerevisiae] [DB:pir2] [MP:13L]> |
| | | | | | | | gp:[GI:1326005] [LN:SC8054] [AC:Z48430:Z71257] |
| | | | | | | | [OR:Saccharomyces cerevisiae] [SR:baker's yeast] [DB:genpept- |
| | | | | | | | pln4] [DE:S.cerevisiae chromosome XIII cosmid 8054.] |
| | | | | | | | [NT:YM8054.04, TYB orf, len: 1328, CAI: 0.15; PS00141] |
| | | | | | | | [SP:Q03434] [LE:5422] [RE:9408] [DI:direct] |
| smorf130 | 621 | 1294 | 57 | 18 | 100 | 0.000037 | pir:[LN:S40969] [AC:S40969] [PN:TyB protein] [CL:TyB protein] |
| | | | | | | | [OR:Saccharomyces cerevisiae] [DB:pir2] [MP:3] |
| smorf131 | 622 | 1295 | 3987 | 1328 | 6915 | 0 | pir:[LN:S69957] [AC:S69957] [PN:TyB protein:protein D9481_12_B] |
| | | | | | | | [CL:TyB protein] [OR:Saccharomyces cerevisiae] [DB:pir2] [MP:4R] |
| smorf135 | 623 | 1296 | 3987 | 1328 | 6939 | 0 | sp:[LN:YME4_YEAST] [AC:Q04711] [GN:TY1B: YML044W: |
| | | | | | | | YM9827.08] [OR:Saccharomyces cerevisiae] [SR:,Baker's yeast] |
| | | | | | | | [DE:TRANSPOSON TY1 PROTEIN B] [SP:Q04711] [DB:swissprot]> |
| | | | | | | | pir:[LN:S50948] [AC:S50948] [PN:TyB protein:protein |
| | | | | | | | YM9827.08:protein YML045w] [CL:TyB protein] [OR:Saccharomyces |
| | | | | | | | cerevisiae] [DB:pir2] [MP:13L]>gp:[GI:1326015] [LN:SC9827] |
| | | | | | | | [AC:Z47816:Z71257] [GN:TYB] [OR:Saccharomyces cerevisiae] |
| | | | | | | | [SR:baker's yeast] [DB:genpept-pln4] [DE:S.cerevisiae chromosome |
| | | | | | | | XIII cosmid 9827.] [NT:YM9827.08, TYB orf, len: 1328, CAI: 0.15; |
| | | | | | | | PS00017] [SP:Q04711] [LE:13801] [RE:17787] [DI:direct] |
| smorf141 | 624 | 1297 | 294 | 97 | 477 | 4.2E−45 | sp:[LN:YRA1_YEAST] [AC:Q12159] [GN:YRA1: YDR381W: |
| | | | | | | | D9481.2: D9509.1] [OR:Saccharomyces cerevisiae] [SR:Baker's |
| | | | | | | | yeast] [DE:RNA ANNEALING PROTEIN YRA1] [SP:Q12159] |
| | | | | | | | [DB:swissprot]>gp:[GI:1912464] [LN:SCU72633] [AC:U72633] |
| | | | | | | | [PN:RNA annealing protein Yra1p] [GN:yra1] [OR:Saccharomyces |
| | | | | | | | cerevisiae] [SR:baker's yeast] [DB:genpept-pln4] |
| | | | | | | | [DE:Saccharomyces cerevisiae RNA annealing protein Yra1p (yra1) |
| | | | | | | | gene, complete cds.] [LE:16:1067] [RE:300:1462] [DI:directJoin] |
| smorf148 | 625 | 1298 | 1398 | 466 | 1696 | 2.8E−174 | pir:[LN:S69641] [AC:S69641] [PN: protein YDR474c] [GN:YDR474c] |
| | | | | | | | [OR:Saccharomyces cerevisiae] [DB:pir2] [MP:4R]>gp:[GI:927751] |
| | | | | | | | [LN:SCD8035] [AC:U33050:Z71256] [PN:Ydr474cp] [GN:YDR474C] |
| | | | | | | | [OR:Saccharomyces cerevisiae] [SR:baker's yeast] [DB:genpept- |
| | | | | | | | pln4] [DE:Saccharomyces cerevisiae chromosome IV cosmids 9410, |
| | | | | | | | 8035, 8166, and 9787.] [NT:similar to Saccharomyces cerevisiae ] |
| | | | | | | | [LE:38195] [RE:39862] [DI:complement] |
| smorf180 | 626 | 1299 | 243 | 80 | 265 | 1.2E−22 | sp:[LN:GOG5_YEAST] [AC:P40107] |
| | | | | | | | [GN:GOG5:VRG4:VAN2:YGL225W] [OR:Saccharomyces |
| | | | | | | | cerevisiae] [SR:,Baker's yeast] [DE:VANADATE RESISTANCE |
| | | | | | | | PROTEIN GOG5/VRG4/VAN2] [SP:P40107] [DB:swissprot]> |
| | | | | | | | pir:[LN:S50238] [AC:S50238:S56042:S59268:S64247] |
| smorf184 | 627 | 1300 | 891 | 296 | 1319 | 2.5E−134 | sp:[LN:CC4_YEAST] [AC:P07834] [GN:CDC4:YFL009W] |
| | | | | | | | [OR:Saccharomyces cerevisiae] [SR:,Baker's yeast] [DE:CELL |
| | | | | | | | DIVISION CONTROL PROTEIN 4] [SP:P07834] [DB:swissprot]> |
| | | | | | | | pir:[LN:S56245] [AC:S56245:S48310:A26867:S62304] [PN:cell |
| | | | | | | | division control protein CDC4:protein YFL009w] [GN:CDC4] |
| | | | | | | | [CL:unassigned WD repeat proteins:WD repeat homology] |
| | | | | | | | [OR:Saccharomyces cerevisiae] [DB:pir2] [MP:6L]>gp:[GI:836745] |
| | | | | | | | [LN:YSCCHRVIN] [AC:D50617: D31600: D44594: D44595: D44596: |
| | | | | | | | D44597: D44598: D44599: D44600] [PN:cell division control protein |
| | | | | | | | 4] [GN:CDC4] [OR:Saccharomyces cerevisiae] [SR:Saccharomyces |
| | | | | | | | cerevisiae (strain:AB972) DNA] [DB:genpept-pln4] |
| | | | | | | | [DE:Saccharomyces cerevisiae chromosome VI complete DNA |
| | | | | | | | sequence.] [NT:YFL009W] [LE:116139] [RE:118478] [DI:direct] |
| smorf202 | 628 | 1301 | 1059 | 352 | 1301 | 2E−132 | sp:[LN:YFA6_YEAST] [AC:P43584] [GN:YFL006W] |
| | | | | | | | [OR:Saccharomyces cerevisiae] [SR:,Baker's yeast] [DE: 28.8 KDA |
| | | | | | | | PROTEIN IN SMC1-SEC4 INTERGENIC REGION] [SP:P43584] |
| | | | | | | | [DB:swissprot]>pir:[LN:S56248] [AC:S56248: S62288: S61731] [PN: |
| | | | | | | | membrane protein YFL006w: protein F001] [OR:Saccharomyces |
| | | | | | | | cerevisiae] [DB:pir2] [MP:6L]>gp:[GI:836748] [LN:YSCCHRVIN] |
| | | | | | | | [AC:D50617: D31600: D44594: D44595: D44596: D44597: D44598: |
| | | | | | | | D44599: D44600] [OR:Saccharomyces cerevisiae] |
| | | | | | | | [SR:Saccharomyces cerevisiae (strain:AB972) DNA] [DB:genpept- |
| | | | | | | | pln4] [DE:Saccharomyces cerevisiae chromosome VI complete DNA |
| | | | | | | | sequence.] [NT:YFL006W] [LE:129140] [RE:129904] [DI:direct] |
| smorf208 | 629 | 1302 | 840 | 279 | 895 | 2.1E−89 | sp:[LN:YFL5_YEAST] [AC:P43617] [GN:YFR045W] |
| | | | | | | | [OR:Saccharomyces cerevisiae] [SR:,Baker's yeast] [DE: |
| | | | | | | | MITOCHONDRIAL CARRIER YFR045W] [SP:P43617] |
| | | | | | | | [DB:swissprot]>pir:[LN:S56300] [AC:S56300:S62256:S63792] [PN: |
| | | | | | | | protein YFR045w: protein R014] [CL: protein YFR045w:ADP, ATP |
| | | | | | | | carrier protein repeat homology] [OR:Saccharomyces cerevisiae] |
| | | | | | | | [DB:pir2] [MP:6R]>gp:[GI:836800] [LN:YSCCHRVIN] [AC:D50617: |
| | | | | | | | D31600:D44594:D44595:D44596:D44597:D44598:D44599: |
| | | | | | | | D44600] [OR:Saccharomyces cerevisiae] [SR:Saccharomyces |
| | | | | | | | cerevisiae (strain:AB972) DNA] [DB:genpept-pln4] |
| | | | | | | | [DE:Saccharomyces cerevisiae chromosome VI complete DNA |
| | | | | | | | sequence.] [NT:YFR045W] [LE:242450] [RE:242986] [DI:direct] |
| smorf212 | 630 | 1303 | 240 | 79 | 290 | 2.7E−25 | sp:[LN:YGW1_YEAST] [AC:P53088:Q92322] [GN:YGL211W] |
| | | | | | | | [OR:Saccharomyces cerevisiae] [SR:,Baker's yeast] [DE: 35.5 KDA |
| | | | | | | | PROTEIN IN VAM7-YPT32 INTERGENIC REGION] |
| | | | | | | | [SP:P53088:Q92322] [DB:swissprot]>pir:[LN:S64230] |
| | | | | | | | [AC:S71668:S71671:S64230] [PN: protein YGL211w: protein |
| | | | | | | | G1125] [CL:conserved protein MJ1157] [OR:Saccharomyces |
| | | | | | | | cerevisiae] [DB:pir2] [MP:7L]>gp:[GI:1655726] [LN:SCU33754] |
| | | | | | | | [AC:U33754] [PN: ] [OR:Saccharomyces cerevisiae] [SR:baker's |
| | | | | | | | yeast strain=S288C-27] [DB:genpept-pln4] [DE:Saccharomyces |
| | | | | | | | cerevisiae Vam7p (VAM7), ras-like GTPase (YPT11) andMIG1-like |
| | | | | | | | zinc finger protein (MLZ1) genes, complete cds and Sip2p(SPM2) |
| | | | | | | | gene, partial cds.] [NT:orf-1] [LE:2003] [RE:2956] [DI:direct] |
| smorf220 | 631 | 1304 | 657 | 218 | 889 | 9.2E−89 | sp:[LN:YGT3_YEAST] [AC:P53102] [GN:YGL183C:G1604] |
| | | | | | | | [OR:Saccharomyces cerevisiae] [SR:,Baker's yeast] [DE: 20.8 KDA |
| | | | | | | | PROTEIN IN COX4-GTS1 INTERGENIC REGION] [SP:P53102] |
| | | | | | | | [DB:swissprot]>pir:[LN:S61134] [AC:S61134:S64200] [PN: protein |
| | | | | | | | YGL183c: protein G1604] [OR:Saccharomyces cerevisiae] [DB:pir2] |
| | | | | | | | [MP:7L]>gp:[GI:1143564] [LN:SCVIIGENE] [AC:X91489] [PN: HMG |
| | | | | | | | box] [GN:G1604] [OR:Saccharomyces cerevisiae] [SR:baker's yeast] |
| | | | | | | | [DB:genpept-pln4] [DE:S.cerevisiae DNA from chromosome VII |
| | | | | | | | including CDC55, RPS26A, COX4, G1380, G1601, G1604, G1607, |
| | | | | | | | LSR1 and G1615 genes.] [SP:P53102] [LE:9998] [RE:10522] |
| | | | | | | | [DI:complement]>gp:[GI:1322797] [LN:SCYGL183C] |
| | | | | | | | [AC:Z72705:Y13135] [OR:Saccharomyces cerevisiae] [SR:baker's |
| | | | | | | | yeast] [DB:genpept-pln4] [DE:S.cerevisiae chromosome VII reading |
| | | | | | | | frame ORF YGL183c.] [NT:ORF YGL183c] [SP:P53102] [LE:531] |
| | | | | | | | [RE:1055] [DI:complement] |
| smorf228 | 632 | 1305 | 582 | 193 | 617 | 6.1E−60 | gp:[GI:13940380] [LN:ZRO303361] [AC:AJ303361] [PN: protein] |
| | | | | | | | [GN:orf] [FN: ] [OR:Zygosaccharomyces rouxii] [DB:genpept-pln4] |
| | | | | | | | [DE:Zygosaccharomyces rouxii gl001-c gene for C-3 |
| | | | | | | | steroldehydrogenase and ORF.] [LE:2022:2324:2863] |
| | | | | | | | [RE:2254:2802:2885] [DI:complementJoin] |
| smorf232 | 633 | 1306 | 3987 | 1328 | 6916 | 0 | pir:[LN:S69838] [AC:S69838] [PN:TyB protein: protein G4054] |
| | | | | | | | [CL:TyB protein] [OR:Saccharomyces cerevisiae] [DB:pir2] [MP:7R]> |
| | | | | | | | gp:[GI:1325964] [LN:SCYGR027C] [AC:Z72812:Y13135] |
| | | | | | | | [GN:TY1B] [OR:Saccharomyces cerevisiae] [SR:baker's yeast] |
| | | | | | | | [DB:genpept-pln4] [DE:S.cerevisiae chromosome VII reading frame |
| | | | | | | | ORF YGR027c.] [LE:2236:3539] [RE:3537:7504] [DI:directJoin]> |
| | | | | | | | gp:[GI:1323003] [LN:SCYGR028W] [AC:Z72813:Y13135] |
| | | | | | | | [GN:TY1B] [OR:Saccharomyces cerevisiae] [SR:baker's yeast] |
| | | | | | | | [DB:genpept-pln4] [DE:S.cerevisiae chromosome VII reading frame |
| | | | | | | | ORF YGR028w.] [LE:1599:2902] [RE:2900:6867] [DI:directJoin] |
| smorf246 | 634 | 1307 | 3813 | 1270 | 6631 | 0 | gp:[GI:536873] [LN:YSCTY31A] [AC:M34549] [GN:POL3] |
| | | | | | | | [OR:Saccharomyces cerevisiae] [SR:baker's yeast] [DB:genpept- |
| | | | | | | | pln4] [DE:Saccharomyces cerevisiae tRNA-Cys gene, complete |
| | | | | | | | sequence; 5′sigmaelement long terminal repeat, complete |
| | | | | | | | sequence; gag3 (gag3) gene, complete cds; POL3 (POL3) gene, |
| | | | | | | | partial cds; and 3′sigma elementlong terminal repeat, complete |
| | | | | | | | sequence.] [LE:<1368] [RE:5180] [DI:direct] |
| smorf248 | 635 | 1308 | 3987 | 1328 | 6909 | 0 | pir:[LN:S45736] [AC:S45736:S45735] [PN:TyB protein:protein |
| | | | | | | | YBL004w-a:protein YBL0325] [CL:TyB protein] [OR:Saccharomyces |
| | | | | | | | cerevisiae] [DB:pir2] [MP:2L]>gp:[GI:535981] [LN:SCYBL004W] |
| | | | | | | | [AC:Z35765:Y13134] [GN:TY1B] [OR:Saccharomyces cerevisiae] |
| | | | | | | | [SR:baker's yeast] [DB:genpept-pln4] [DE:S.cerevisiae chromosome |
| | | | | | | | II reading frame ORF YBL004w.] [LE:933:2239] [RE:2237:6201] |
| | | | | | | | [DI:directJoin]>gp:[GI:535986] [LN:SCYBL005W] |
| | | | | | | | [AC:Z35766:Y13134] [GN:TY1B] [OR:Saccharomyces cerevisiae] |
| | | | | | | | [SR:baker's yeast] [DB:genpept-pln4] [DE:S.cerevisiae chromosome |
| | | | | | | | II reading frame ORF YBL005w.] [LE:4201:5507] [RE:5505:9469] |
| | | | | | | | [DI :directJoin] |
| smorf259 | 636 | 1309 | 1194 | 397 | 1920 | 5.1E−198 | pir:[LN:S50953] [AC:S50953:S50954:S64818] [PN: protein |
| | | | | | | | YLL066c: protein L0519: protein L0532] [OR:Saccharomyces |
| | | | | | | | cerevisiae] [DB:pir2] [MP:12L]>gp:[GI:642317] [LN:SCCH13LST] |
| | | | | | | | [AC:Z47973] [PN:ORF L0519] [OR:Saccharomyces cerevisiae] |
| | | | | | | | [SR:baker's yeast] [DB:genpept-pln4] [DE:S.cerevisiae chromosome |
| | | | | | | | XII DNA including subtelomeric region ofleft arm.] [LE:3110:6540] |
| | | | | | | | [RE:6440:6826] [DI:complementJoin]>gp:[GI:1360282] |
| | | | | | | | [LN:SCYLL066C] [AC:Z73171:Y13138] [OR:Saccharomyces |
| | | | | | | | cerevisiae] [SR:baker's yeast] [DB:genpept-pln4] [DE:S.cerevisiae |
| | | | | | | | chromosome XII reading frame ORF YLL066c.] [NT:ORF YLL066c] |
| | | | | | | | [LE:3110:6540] [RE:6440:6826] [DI:complementJoin] |
| smorf261 | 637 | 1310 | 4398 | 1465 | 7520 | 0 | pir:[LN:S31262] [AC:S31262] [PN:TyB protein] [CL:TyB protein] |
| | | | | | | | [OR:Saccharomyces cerevisiae] [DB:pir2] |
| smorf267 | 638 | 1311 | 486 | 161 | 718 | 1.2E−70 | pir:[LN:S52597] [AC:S52597] [PN: membrane protein YHR070c-a] |
| | | | | | | | [GN:YHR070c-a] [CL:Saccharomyces cerevisiae membrane protein |
| | | | | | | | YHR070c-a] [OR:Saccharomyces cerevisiae] [DB:pir2] [MP:8R] |
| smorf277 | 639 | 1312 | 1167 | 389 | 1721 | 6.3E−177 | sp:[LN:YHR5_YEAST] [AC:P38823] [GN:YHR115C] |
| | | | | | | | [OR:Saccharomyces cerevisiae] [SR:,Baker's yeast] [DE: 46.1 KDA |
| | | | | | | | PROTEIN IN ERP5-ORC6 INTERGENIC REGION] [SP:P38823] |
| | | | | | | | [DB:swissprot]>pir:[LN:S48957] [AC:S48957] [PN: protein |
| | | | | | | | YHR115c] [OR:Saccharomyces cerevisiae] [DB:pir2] [MP:8R]> |
| | | | | | | | gp:[GI:529132] [LN:YSCH8263] [AC:U00059:U00093] |
| | | | | | | | [PN:Yhr115cp] [GN:YHR115c] [OR:Saccharomyces cerevisiae] |
| | | | | | | | [SR:baker's yeast strain=S288C (AB972)] [DB:genpept-pln4] |
| | | | | | | | [DE:Saccharomyces cerevisiae chromosome VIII cosmid 8263.] |
| | | | | | | | [LE:26661] [RE:27911] [DI:complement] |
| smorf292 | 640 | 1313 | 1305 | 434 | 2215 | 2.8E−229 | pir:[LN:S50953] [AC:S50953: S50954: S64818] [PN: protein |
| | | | | | | | YLL066c: protein L0519: protein L0532] [OR:Saccharomyces |
| | | | | | | | cerevisiae] [DB:pir2] [MP:12L]>gp:[GI:642317] [LN:SCCH13LST] |
| | | | | | | | [AC:Z47973] [PN:ORF L0519] [OR:Saccharomyces cerevisiae] |
| | | | | | | | [SR:baker's yeast] [DB:genpept-pln4] [DE:S.cerevisiae chromosome |
| | | | | | | | XII DNA including subtelomeric region ofleft arm.] [LE:3110:6540] |
| | | | | | | | [RE:6440:6826] [DI:complementJoin]>gp:[GI:1360282] |
| | | | | | | | [LN:SCYLL066C] [AC:Z73171:Y13138] [OR:Saccharomyces |
| | | | | | | | cerevisiae] [SR:baker's yeast] [DB:genpept-pln4] [DE:S.cerevisiae |
| | | | | | | | chromosome XII reading frame ORF YLL066c.] [NT:ORF YLL066c] |
| | | | | | | | [LE:3110:6540] [RE:6440:6826] [DI:complement Join] |
| smorf302 | 641 | 1314 | 1035 | 344 | 1533 | 5.2E−157 | sp:[LN:BET4_YEAST] [AC:Q00618] [GN:BET4:YJL031C:J1254] |
| | | | | | | | [OR:Saccharomyces cerevisiae] [SR:Baker's yeast] [EC:2.5.1.-] |
| | | | | | | | [DE:SUBUNIT)] [SP:Q00618] [DB:swissprot]>pir:[LN:S48301] |
| | | | | | | | [AC:S48301:A39655:S56803:S19037] |
| smorf306 | 642 | 1315 | 1116 | 371 | 1632 | 1.7E−167 | sp:[LN:YJY3_YEAST] [AC:P47088] [GN:YJR013W:J1444:YJR83.11] |
| | | | | | | | [OR:Saccharomyces cerevisiae] [SR:,Baker's yeast] [DE: 35.6 KDA |
| | | | | | | | PROTEIN IN SPC1-ILV3 INTERGENIC REGION] [SP:P47088] |
| | | | | | | | [DB:swissprot]>pir:[LN:S55201] [AC:S55201:S57028] [PN: protein |
| | | | | | | | YJR013w: protein J1444: protein YJR83.11] [OR:Saccharomyces |
| | | | | | | | cerevisiae] [DB:pir2] [MR:10R]>gp:[GI:854586] [LN:SCXCOSM83] |
| | | | | | | | [AC:X87611] [GN:ORF YJR83.11] [OR:Saccharomyces cerevisiae] |
| | | | | | | | [SR:baker's yeast] [DB:genpept-pln4] [DE:S.cerevisiae chromosome |
| | | | | | | | X DNA (cosmid 83).] [SP:P47088] [LE:33505] [RE:34422] [DI:direct]> |
| | | | | | | | gp:[GI:1015644] [LN:SCYJR013W] [AC:Z49513:Y13136] |
| | | | | | | | [OR:Saccharomyces cerevisiae] [SR:baker's yeast] [DB:genpept- |
| | | | | | | | pln4] [DE:S.cerevisiae chromosome X reading frame ORF |
| | | | | | | | YJR013w.] [NT:ORF YJR013w] [SP:P47088] [LE:259] [RE:1176] |
| | | | | | | | [DI:direct] |
| smorf310 | 643 | 1316 | 198 | 65 | 114 | 0.0000012 | gp:[GI:1098486] [LN:SCU12141] [AC:U12141] [PN:Ynl2444p] |
| | | | | | | | [GN:YNL2444c] [OR:Saccharomyces cerevisiae] [SR:baker's yeast] |
| | | | | | | | [DB:genpept-pln4] [DE:Saccharomyces cerevisiae chromosome XIV |
| | | | | | | | left arm fragment.] [NT:mitochondrial transit peptide] [LE:21823] |
| | | | | | | | [RE:22185] [DI:complement] |
| smorf319 | 644 | 1317 | 267 | 88 | 344 | 5.2E−31 | sp:[LN:AADE_YEAST] [AC:P42884] [GN:AAD14:YNL331C:N0300] |
| | | | | | | | [OR:Saccharomyces cerevisiae] [SR:,Baker's yeast] [EC:1.1.1.-] |
| | | | | | | | [DE: ARYL-ALCOHOL DEHYDROGENASE AAD14,] [SP:P42884] |
| | | | | | | | [DB:swissprot]>pir:[LN:S51335] |
| | | | | | | | [AC:S51335:S57392:S63314:S63317] |
| smorf322 | 645 | 1318 | 105 | 34 | 124 | 0.0000015 | gp:[GI:2980815] [LN:SCYKL200C] [AC:Z28200:Y13137] [GN:MNN4] |
| | | | | | | | [OR:Saccharomyces cerevisiae] [SR:baker's yeast] [DB:genpept- |
| | | | | | | | pln4] [DE:S.cerevisiae chromosome XI reading frame ORF |
| | | | | | | | YKL200c.] [NT:ORF YKL201c] [LE:<1] [RE:1917] [DI:complement] |
| smorf336 | 646 | 1319 | 639 | 212 | 751 | 3.8E−74 | sp:[LN:YKA2_YEAST] [AC:P36108] [GN:YKL002W] |
| | | | | | | | [OR:Saccharomyces cerevisiae] [SR:,Baker's yeast] [DE: 16.7 KDA |
| | | | | | | | PROTEIN MRP17-MET14 INTERGENIC REGION] [SP:P36108] |
| | | | | | | | [DB:swissprot]>pir:[LN:S37812] [AC:S37812:S37813] [PN: protein |
| | | | | | | | YKL002w] [OR:Saccharomyces cerevisiae] [DB:pir2] [MP:11L]> |
| | | | | | | | gp:[GI:485989] [LN:SCYKL002W] [AC:Z28002:Y13137] |
| | | | | | | | [OR:Saccharomyces cerevisiae] [SR:baker's yeast] [DB:genpept- |
| | | | | | | | pln4] [DE:S.cerevisiae chromosome XI reading frame ORF |
| | | | | | | | YKL002w.] [NT:ORF YKL002W] [SP:P36108] [LE:597] [RE:1052] |
| | | | | | | | [DI:direct] |
| smorf340 | 647 | 1320 | 1314 | 438 | 2278 | 5.9E−236 | sp:[LN:GLG1_YEAST] [AC:P36143] [GN:GLG1:YKR058W] |
| | | | | | | | [OR:Saccharomyces cerevisiae] [SR:,Baker's yeast] |
| | | | | | | | [DE:GLYCOGEN SYNTHESIS INITIATOR PROTEIN GLG1] |
| | | | | | | | [SP:P36143] [DB:swissprot]>gp:[GI:902793] [LN:SCU25546] |
| | | | | | | | [AC:U25546] [PN:Glg1p] [GN:GLG1] [OR:Saccharomyces |
| | | | | | | | cerevisiae] [SR:baker's yeast] [DB:genpept-pln4] |
| | | | | | | | [DE:Saccharomyces cerevisiae self-glucosylating initiator of |
| | | | | | | | glycogensynthesis (GLG1) gene, complete cds.] [NT:self- |
| | | | | | | | glucosylating initiator of glycogen synthesis;] [LE:1] [RE:1857] |
| | | | | | | | [DI:direct] |
| smorf355 | 648 | 1321 | 3987 | 1328 | 6917 | 0 | pir:[LN:S50663] [AC:S50663:S30812:S53556] [PN:TyB |
| | | | | | | | protein:protein YER160c] [CL:TyB protein] [OR:Saccharomyces |
| | | | | | | | cerevisiae] [DB:pir2] [MP:5R]>gp:[GI:603400] [LN:SCE8229] |
| | | | | | | | [AC:U18917:L10718:U00092] [PN:Yer160cp] [GN:YER160C] |
| | | | | | | | [OR:Saccharomyces cerevisiae] [SR:baker's yeast] [DB:genpept- |
| | | | | | | | pln4] [DE:Saccharomyces cerevisiae chromosome V cosmids 8229, |
| | | | | | | | 9115, 9132, 9981, and lambda clones 7990 and 6134.] |
| | | | | | | | [NT:transposon Ty with frame shift at] [LE:50840:54807] |
| | | | | | | | [RE:54805:56108] [DI:complement Join] |
| smorf356 | 649 | 1322 | 1641 | 547 | 1748 | 8.6E−180 | pir:[LN:S61628] [AC:S61628:S64882] [PN: protein YLR054c: |
| | | | | | | | protein L2141] [CL:Saccharomyces cerevisiae protein YLR054c] |
| | | | | | | | [OR:Saccharomyces cerevisiae] [DB:pir2] [MP:12R]> |
| | | | | | | | gp:[GI:1181275] [LN:SCLACHXII] [AC:X94607] [GN:L2141] |
| | | | | | | | [OR:Saccharomyces cerevisiae] [SR:baker's yeast] [DB:genpept- |
| | | | | | | | pln4] [DE:S.cerevisiae (EU) DNA from left arm of chromosome XII.] |
| | | | | | | | [LE:15053] [RE:16591] [DI:complement]>gp:[GI:1360394] |
| | | | | | | | [LN:SCYLR054C] [AC:Z73226:Y13138] [OR:Saccharomyces |
| | | | | | | | cerevisiae] [SR:baker's yeast] [DB:genpept-pln4] [DE:S.cerevisiae |
| | | | | | | | chromosome XII reading frame ORF YLR054c.] [NT:ORF YLR054C] |
| | | | | | | | [LE:291] [RE:1829] [DI:complement] |
| smorf500 | 650 | 1323 | 3987 | 1328 | 6907 | 0 | pir:[LN:S69963] [AC:S69963] [PN:TyB protein:protein L8083_11_c] |
| | | | | | | | [CL:TyB protein] [OR:Saccharomyces cerevisiae] [DB:pir2] [MP:12R] |
| smorf502 | 651 | 1324 | 987 | 328 | 1707 | 1.9E−175 | gp:[GI:1204150] [LN:SC8142A] [AC:Z68194:Z71256] [PN: ] [GN:TyB] |
| | | | | | | | [OR:Saccharomyces cerevisiae] [SR:baker's yeast] [DB:genpept- |
| | | | | | | | pln4] [DE:S.cerevisiae chromosome IV cosmid 8142A.] [NT:Protein |
| | | | | | | | sequence is in conflict with the conceptual] [LE:20534] [RE:24520] |
| | | | | | | | [DI:complement]>gp:[GI:1122342] [LN:SC8142B] [AC:Z68195] [PN: |
| | | | | | | | ] [GN:TyB] [OR:Saccharomyces cerevisiae] [SR:baker's yeast] |
| | | | | | | | [DB:genpept-pln4] [DE:S.cerevisiae chromosome IV cosmid 8142B.] |
| | | | | | | | [NT:Protein sequence is in conflict with the conceptual] [LE:796] |
| | | | | | | | [RE:4782] [DI:complement] |
| smorf503 | 652 | 1325 | 3018 | 1005 | 5233 | 0 | pir:[LN:S69957] [AC:S69957] [PN:TyB protein:protein D9481_12_B] |
| | | | | | | | [CL:TyB protein] [OR:Saccharomyces cerevisiae] [DB:pir2] [MP:4R] |
| smorf518 | 653 | 1326 | 4044 | 1347 | 7029 | 0 | pir:[LN:S69966] [AC:S69966] [PN:TyB protein:protein L9931_7_b] |
| | | | | | | | [CL:TyB protein] [OR:Saccharomyces cerevisiae] [DB:pir2] [MP:12R] |
| smorf520 | 654 | 1327 | 342 | 113 | 447 | 6.3E−42 | pir:[LN:S78568] [AC:S78568] [PN:snRNP protein SMX4:protein |
| | | | | | | | YLR438c-a:small nuclear protein SMX4] [GN:SMX4:YLR438c-a] |
| | | | | | | | [OR:Saccharomyces cerevisiae] [DB:pir2] [MP:12L] |
| smorf537 | 655 | 1328 | 1449 | 483 | 2531 | 9.2E−263 | sp:[LN:HFA1_YEAST] [AC:P32874] |
| | | | | | | | [GN:HFA1:YMR207C:YM8261.01C:YM8325.08C] |
| | | | | | | | [OR:Saccharomyces cerevisiae] [SR:,Baker's yeast] [DE:HFA1 |
| | | | | | | | PROTEIN] [SP:P32874] [DB:swissprot] |
| smorf546 | 656 | 1329 | 1689 | 562 | 2893 | 4E−301 | sp:[LN:GAS1_YEAST] [AC:P22146:P23151] |
| | | | | | | | [GN:GAS1:GGP1:YMR307W:YM9952.09] [OR:Saccharomyces |
| | | | | | | | cerevisiae] [SR:,Baker's yeast] [DE:GLYCOLIPID ANCHORED |
| | | | | | | | SURFACE PROTEIN PRECURSOR (GLYCOPROTEIN GP115)] |
| | | | | | | | [SP:P22146:P23151] [DB:swissprot]>pir:[LN:RWBYS1] |
| smorf551 | 657 | 1330 | 393 | 130 | 348 | 2E−31 | sp:[LN:YH17_YEAST] [AC:P38898] [GN:YHR217C] |
| | | | | | | | [OR:Saccharomyces cerevisiae] [SR:,Baker's yeast] [DE: 17.1 KDA |
| | | | | | | | PROTEIN IN PUR5 3'REGION] [SP:P38898] [DB:swissprot]> |
| | | | | | | | pir:[LN:S48998] [AC:S48998] [PN: protein YHR217c] |
| | | | | | | | [GN:YHR217c] [OR:Saccharomyces cerevisiae] [DB:pir2] [MP:8R]> |
| | | | | | | | gp:[GI:551324] [LN:YSCH9177] [AC:U00029:U00093] |
| | | | | | | | [PN:Yhr217cp] [GN:YHR217c] [OR:Saccharomyces cerevisiae] |
| | | | | | | | [SR:baker's yeast strain=S288C (AB972)] [DB:genpept-pln4] |
| | | | | | | | [DE:Saccharomyces cerevisiae chromosome VIII cosmid 9177.] |
| | | | | | | | [LE:50035] [RE:50496] [DI:complement] |
| smorf553 | 658 | 1331 | 1335 | 444 | 2334 | 6.9E−242 | pir:[LN:S69970] [AC:S69970] [PN:TyA protein:protein N0569] |
| | | | | | | | [CL:TyA protein] [OR:Saccharomyces cerevisiae] [DB:pir2] [MP:14L]> |
| | | | | | | | gp:[GI:1302360] [LN:SCYNL284C] [AC:Z71560:Y13139] |
| | | | | | | | [GN:TY1A] [OR:Saccharomyces cerevisiae] [SR:baker's yeast] |
| | | | | | | | [DB:genpept-pln4] [DE:S.cerevisiae chromosome XIV reading frame |
| | | | | | | | ORF YNL284c.] [LE:4598] [RE:5920] [DI:complement]> |
| | | | | | | | gp:[GI:1302365] [LN:SCYNL285W] [AC:Z71561:Y13139] |
| | | | | | | | [GN:TY1A] [OR:Saccharomyces cerevisiae] [SR:baker's yeast] |
| | | | | | | | [DB:genpept-pln4] [DE:S.cerevisiae chromosome XIV reading frame |
| | | | | | | | ORF YNL285w.] [LE:4830] [RE:6152] [DI:complement] |
| smorf565 | 659 | 1332 | 3969 | 1322 | 6876 | 0 | pir:[LN:S69972] [AC:S69972] [PN:TyB protein:protein N2453] |
| | | | | | | | [CL:TyB protein] [OR:Saccharomyces cerevisiae] [DB:pir2] [MP:14L]> |
| | | | | | | | gp:[GI:1301920] [LN:SCYNL054W] [AC:Z71330:Y13139] |
| | | | | | | | [GN:TY1B] [OR:Saccharomyces cerevisiae] [SR:baker's yeast] |
| | | | | | | | [DB:genpept-pln4] [DE:S.cerevisiae chromosome XIV reading frame |
| | | | | | | | ORF YNL054w.] [LE:611:1917] [RE:1915:5861] [DI:directJoin]> |
| | | | | | | | gp:[GI:1301925] [LN:SCYNL055C] [AC:Z71331:Y13139] |
| | | | | | | | [GN:TY1B] [OR:Saccharomyces cerevisiae] [SR:baker's yeast] |
| | | | | | | | [DB:genpept-pln4] [DE:S.cerevisiae chromosome XIV reading frame |
| | | | | | | | ORF YNL055c.] [LE:1614:2920] [RE:2918:6864] [DI:directJoin] |
| smorf566 | 660 | 1333 | 1335 | 444 | 2331 | 1.4E−241 | pir:[LN:S69971] [AC:S69971] [PN:TyA protein:protein N2447] |
| | | | | | | | [CL:TyA protein] [OR:Saccharomyces cerevisiae] [DB:pir2] [MP:14L]> |
| | | | | | | | gp:[GI:1301919] [LN:SCYNL054W] [AC:Z71330:Y13139] |
| | | | | | | | [GN:TY1A] [OR:Saccharomyces cerevisiae] [SR:baker's yeast] |
| | | | | | | | [DB:genpept-pln4] [DE:S.cerevisiae chromosome XIV reading frame |
| | | | | | | | ORF YNL054w.] [LE:611] [RE:1933] [DI:direct]>gp:[GI:1301924] |
| | | | | | | | [LN:SCYNL055C] [AC:Z71331:Y13139] [GN:TY1A] |
| | | | | | | | [OR:Saccharomyces cerevisiae] [SR:baker's yeast] [DB:genpept- |
| | | | | | | | pln4] [DE:S.cerevisiae chromosome XIV reading frame ORF |
| | | | | | | | YNL055c.] [LE:1614] [RE:2936] [DI:direct] |
| smorf579 | 661 | 1334 | 753 | 250 | 891 | 5.6E−89 | pir:[LN:S66862] [AC:S66862] [PN: membrane protein YOL163w: |
| | | | | | | | protein O0230] [GN:YOL163w] [OR:Saccharomyces cerevisiae] |
| | | | | | | | [DB:pir2] [MP:15L]>gp:[GI:1420080] [LN:SCYOL163W] |
| | | | | | | | [AC:Z74905:Y13140] [OR:Saccharomyces cerevisiae] [SR:baker's |
| | | | | | | | yeast] [DB:genpept-pln4] [DE:S.cerevisiae chromosome XV reading |
| | | | | | | | frame ORE YOL163w.] [NT:ORF YOL163w] [LE:1481] [RE:1990] |
| | | | | | | | [DI:direct] |
| smorf588 | 662 | 1335 | 1635 | 545 | 2720 | 8.6E−283 | pir:[LN:S77690] [AC:S77690:S66767:S66768] [PN: membrane |
| | | | | | | | protein YOL075c: protein O1125: protein O1130: protein YOL074c] |
| | | | | | | | [CL:unassigned ATP-binding cassette proteins:ATP-binding cassette |
| | | | | | | | homology] [OR:Saccharomyces cerevisiae] [DB:pir2] [MP:15L] |
| smorf595 | 663 | 1336 | 2010 | 669 | 3365 | 0 | sp:[LN:VPS5_YEAST] [AC:Q92331:Q08483] |
| | | | | | | | [GN:VPS5:GRD2:YOR069W:YOR29-20] [OR:Saccharomyces |
| | | | | | | | cerevisiae] [SR:,Baker's yeast] [DE:VACUOLAR PROTEIN |
| | | | | | | | SORTING-ASSOCIATED PROTEIN VPS5] [SP:Q92331:Q08483] |
| | | | | | | | [DB:swissprot]>gp:[GI:1657952] [LN:SCU73512] [AC:U73512] |
| | | | | | | | [PN:Vps5p] [GN:VPS5] [FN:Golgi retention and vacuolar protein |
| | | | | | | | sorting] [OR:Saccharomyces cerevisiae] [SR:baker's yeast] |
| | | | | | | | [DB:genpept-pln4] [DE:Saccharomyces cerevisiae Vps5p (VPS5) |
| | | | | | | | gene, complete cds.] [NT:sorting nexin family member; Grd2p] |
| | | | | | | | [LE:290] [RE:2317] [DI:direct]>gp:[GI:1814080] [LN:SCU84735] |
| | | | | | | | [AC:U84735] [PN:Vps59] [GN:VPS5] [FN:vacuolar protein sorting] |
| | | | | | | | [OR:Saccharomyces cerevisiae] [SR:baker's yeast] [DB:genpept- |
| | | | | | | | pln4] [DE:Saccharomyces cerevisiae sorting nexin homolog Vps5p |
| | | | | | | | (VPS5) gene, complete cds.] [NT:sorting nexin homolog] [LE:501] |
| | | | | | | | [RE:2528] [DI:direct] |
| smorf600 | 664 | 1337 | 1023 | 340 | 1638 | 3.9E−168 | sp:[LN:TYSY_YEAST] [AC:P06785:Q12694] |
| | | | | | | | [GN:TMP1:CDC21:YOR074C:YOR29-25] [OR:Saccharomyces |
| | | | | | | | cerevisiae] [SR:,Baker's yeast] [EC:2.1.1.45] [DE:THYMIDYLATE |
| | | | | | | | SYNTHASE, (TS)] [SP:P06785:Q12694] [DB:swissprot]> |
| | | | | | | | gp:(GI:2104886] [LN:SCXV55KB] [AC:Z70678] [GN:YOR29-25] |
| | | | | | | | [OR:Saccharomyces cerevisiae] [SR:baker's yeast] [DB:genpept- |
| | | | | | | | pln4] [DE:S.cerevisiae chromosome XV DNA, 54.7 kb region.] |
| | | | | | | | [SP:P06785] [LE:43507] [RE:44421] [DI:complement]> |
| | | | | | | | gp:[GI:172990] [LN:YSCTIMP1A] [AC:J02706] [PN:thymidylate |
| | | | | | | | synthase] [GN:TIMP1] [OR:Saccharomyces cerevisiae] |
| | | | | | | | [SR:Saccharomyces cerevisiae DNA] [DB:genpept-pln4] |
| | | | | | | | [DE:Saccharomyces cerevisiae thymidylate sythase (TIMP1) gene, |
| | | | | | | | completecds.] [LE:498] [RE:1412] [DI:direct] |
| smorf604 | 665 | 1338 | 3987 | 1328 | 6918 | 0 | pir:[LN:S61763] [AC:S61763:S69977] [PN:TyB protein:protein |
| | | | | | | | O3367:protein YOR3367w] [CL:TyB protein] [OR:Saccharomyces |
| | | | | | | | cerevisiae] [DB:pir2] [MP:15R]>gp:[GI:1164985] [LN:SC130KBXV] |
| | | | | | | | [AC:X94335] [GN:YOR3367w] [OR:Saccharomyces cerevisiae] |
| | | | | | | | [SR:baker's yeast] [DB:genpept-pln4] [DE:S.cerevisiae 130 kb DNA |
| | | | | | | | fragment from chromosome XV.] [NT:Ty retroposon like peptide with + |
| | | | | | | | 1 frameshift] [LE:118636:119942] [RE:119940:123904] |
| | | | | | | | [DI:directJoin]>gp:[GI:1420360] [LN:SCYOR142W] |
| | | | | | | | [AC:Z75050:Y13140] [GN:TY1B] [OR:saccharomyces cerevisiae] |
| | | | | | | | [SR:baker's yeast] [DB:genpept-pln4] [DE.S.cerevisiae chromosome |
| | | | | | | | XV reading frame ORF YOR142w.] [LE:2525:3831] [RE:3829:7793] |
| | | | | | | | [DI:directJoin]>gp:[GI:1420363] [LN:SCYOR143C] |
| | | | | | | | [AC:Z75051:Y13140] [GN:TY1B] [OR:Saccharomyces cerevisiae] |
| | | | | | | | [SR:baker's yeast] [DB:genpept-pln4] [DE:S.cerevisiae chromosome |
| | | | | | | | XV reading frame ORF YOR143c.] [LE:1066:2372] [RE:2370:6334] |
| | | | | | | | [DI:direct Join] |
| smorf617 | 666 | 1339 | 558 | 185 | 473 | 1.1E−44 | sp:[LN:RS1A_YEAST] [AC:Q08745] [GN:RPS10A:YOR293W] |
| | | | | | | | [OR:Saccharomyces cerevisiae] [SR:,Baker's yeast] [DE:40S |
| | | | | | | | RIBOSOMAL PROTEIN S10-A] [SP:Q08745] [DB:swissprot]> |
| | | | | | | | pir:[LN:S67197] [AC:S67197] [PN:ribosomal protein S10.e.A, |
| | | | | | | | cytosolic:protein O5611:protein YOR293w] [GN:YOR293w] [CL:rat |
| | | | | | | | ribosomal protein S10:ribosomal protein S10 homology] |
| | | | | | | | [OR:Saccharomyces cerevisiae] [DB:pir1] [MP:15R]> |
| | | | | | | | gp:[GI:1420650] [LN:SCYOR293W] [AC:Z75201:Y13140] |
| | | | | | | | [OR:Saccharomyces cerevisiae] [SR:baker's yeast] [DB:genpept- |
| | | | | | | | pln4] [DE:S.cerevisiae chromosome XV reading frame ORF |
| | | | | | | | YOR293w.] [NT:ORF YOR293w] [SP:Q08745] [LE:516:1005] |
| | | | | | | | [RE:567:1270] [DI:direct Join] |
| smorf628 | 667 | 1340 | 4044 | 1347 | 7053 | 0 | gp:[GI:1122340] [LN:SC8142A] [AC:Z68194:Z71256] [PN:] [GN:TyB] |
| | | | | | | | [OR:Saccharomyces cerevisiae] [SR:baker's yeast] [DB:genpept- |
| | | | | | | | pln4] [DE:S.cerevisiae chromosome IV cosmid 8142A.] [NT:Protein |
| | | | | | | | sequence is in conflict with the conceptual] [LE:15257] [RE:19300] |
| | | | | | | | [DI:direct] |
| smorf635 | 668 | 1341 | 5526 | 1841 | 9473 | 0 | sp:[LN:YG67_YEAST] [AC:P53345] [GN:YGR296W, YPL283C] |
| | | | | | | | [OR:Saccharomyces cerevisiae] [SR:,Baker's yeast] [DE: 211.1 |
| | | | | | | | KDA PROTEIN IN MAL1S 3'REGION] [SP:P53345] [DB:swissprot]> |
| | | | | | | | pir:[LN:S64633] [AC:S64633:S64634:S65338:S65337] [PN: |
| | | | | | | | membrane protein YGR296w: protein G9608: protein P0254: |
| | | | | | | | protein YPL283c] [OR:Saccharomyces cerevisiae] [DB:pir2] |
| | | | | | | | [MP:16L]>gp:[GI:1323541] [LN:SCYGR296W] [AC:Z73081:Y13135] |
| | | | | | | | [OR:Saccharomyces cerevisiae] [SR:baker's yeast] [DB:genpept- |
| | | | | | | | pln4] [DE:S.cerevisiae chromosome VII reading frame ORF |
| | | | | | | | YGR296w.] [NT:ORF YGR296w; Y'element] [SP:P53345] |
| | | | | | | | [LE:2135:2302] [RE:2153:7862] [DI:directJoin]>gp:[GI:1370582] |
| | | | | | | | [LN:SCYPL283C] [AC:Z73521:U00094] [OR:Saccharomyces |
| | | | | | | | cerevisiae] [SR:baker's yeast] [DB:genpept-pln4] [DE:S.cerevisiae |
| | | | | | | | chromosome XVI reading frame ORF YPL283c.] [NT:ORF YPL283c] |
| | | | | | | | [SP:P53345] [LE:280:5989] [RE:5840:6007] [DI:complementJoin] |
| smorf641 | 669 | 1342 | 315 | 104 | 466 | 6.1E−44 | sp:[LN:R36B_YEAST] [AC:O14455] |
| | | | | | | | [GN:RPL36B:RPL39B:YPL249BC] [OR:Saccharomyces cerevisiae] |
| | | | | | | | [SR:,Baker's yeast] [DE:60S RIBOSOMAL PROTEIN L36-B (L39B) |
| | | | | | | | (YL39)] [SP:O14455] [DB:swissprot] |
| smorf653 | 670 | 1343 | 2169 | 723 | 3639 | 0 | pir:[LN:S52611] [AC:S52611] [PN:TyB protein:protein YHL008w-a] |
| | | | | | | | [CL:TyB protein] [OR:Saccharomyces cerevisiae] [DB:pir2] [MP:8L] |
| smorf668 | 671 | 1344 | 1737 | 578 | 2965 | 0 | sp:[LN:DBFB_YEAST] [AC:P32328:Q06105] [GN:DBF20:YPR111W] |
| | | | | | | | [OR:Saccharomyces cerevisiae] [SR:Baker's yeast] [EC:2.7.1.-] |
| | | | | | | | [DE:PROTEIN KINASE DBF20] [SP:P32328:Q06105] [DB:swissprot]> |
| | | | | | | | pir:[LN:S59776] [AC:S59776:JQ1276:S19039] [PN:protein kinase |
| | | | | | | | DBF20:protein P8283.6:protein YPR111w] [GN:DBF20] [CL:protein |
| | | | | | | | kinase DBF2:protein kinase homology] [OR:Saccharomyces |
| | | | | | | | cerevisiae] [EC:2.7.1.-] [DB:pir2] [MP:16R] |
| smorf670 | 672 | 1345 | 3987 | 1328 | 6925 | 0 | sp:[LN:YJZ7_YEAST] [AC:P47098:P87194] |
| | | | | | | | [GN:TY1B:YJR027W:J1560] [OR:Saccharomyces cerevisiae] |
| | | | | | | | [SR:Baker's yeast] [DE:TRANSPOSON TY1 PROTEIN B] |
| | | | | | | | [SP:P47098:P87194] [DB:swissprot]>gp:[GI:2131097] |
| | | | | | | | [LN:SCYJR026W] [AC:Z49526:Y13136] [GN:TY1B] |
| | | | | | | | [OR:Saccharomyces cerevisiae] [SR:baker's yeast] [DB:genpept- |
| | | | | | | | pln4] [DE:S.cerevisiae chromosome X reading frame ORF |
| | | | | | | | YJR026w.] [LE:1089:2389] [RE:2387.6357] [DI:direct Join] |
| smorf671 | 673 | 1346 | 3990 | 1329 | 6935 | 0 | sp:[LN:YME4_YEAST] [AC:Q04711] |
| | | | | | | | [GN:TY1B:YML044W:YM9827.08] [OR:Saccharomyces cerevisiae] |
| | | | | | | | [SR:,Baker's yeast] [DE:TRANSPOSON TY1 PROTEIN B] |
| | | | | | | | [SP:Q04711] [DB:swissprot]>pir:[LN:S50948] [AC:S50948] [PN:TyB |
| | | | | | | | protein:protein YM9827.08:protein YML045w] [CL:TyB protein] |
| | | | | | | | [OR:Saccharomyces cerevisiae] [DB:pir2] [MP:13L]> |
| | | | | | | | gp:[GI:1326015] [LN:SC9827] [AC:Z47816:Z71257] [GN:TYB] |
| | | | | | | | [OR:Saccharomyces cerevisiae] [SR:baker's yeast] [DB:genpept- |
| | | | | | | | pln4] [DE:S.cerevisiae chromosome XIII cosmid 9827.] |
| | | | | | | | [NT:YM9827.08, TYB orf, len: 1328, CAI: 0.15; PS00017] |
| | | | | | | | [SP:Q04711] [LE:13801] [RE:17787] [DI:direct] |

TABLE 3
MOTIFS

| | | | | | | | | predicted trans- | | | |
|---|---|---|---|---|---|---|---|---|---|---|---|
| | | | | | | | COILSCAN | membrane | | | |
| | | | LENGTH | | | ADDITIONAL | predicted coil | domains | | PDB hit | |
| SEQID | SEQ ID NO: | smorf# | (aa) | BLIMPS MOTIF DESCRIPTION | PFAM motifs | MOTIFS | structure | TMPRED | p-value | description | p-value |
| SC0001 | SEQ ID NO:1 | smorf003 | 66 | Deoxyribonuclease I | — | — | — | 1 | 0.038 | — | — |
| SC0002 | SEQ ID NO:2 | smorf013 | 100 | Lysyl oxidase | — | — | — | 1 | 3.1E-12 | — | — |
| SC0003 | SEQ ID NO:3 | smorf016 | 203 | Major sperm protein (MSP) | MSP_domain | — | — | — | 1.3E-48 | — | — |
| | | | | domain | | | | | | | |
| SC0004 | SEQ ID NO:4 | smorf018 | 95 | Marek's disease glycoprotein A | — | — | — | — | 4.4E-18 | — | — |
| | | | | signature | | | | | | | |
| SC0005 | SEQ ID NO:5 | smorf019 | 107 | Ornatin signature | — | — | — | — | 6.5E-56 | — | — |
| SC0006 | SEQ ID NO:6 | smorf024 | 85 | Interleukin-1B converting | — | — | — | 1 | — | — | — |
| | | | | enzyme signature | | | | | | | |
| SC0007 | SEQ ID NO:7 | smorf028 | 63 | Aldehyde dehydrogenase | — | — | — | — | 2.9E-28 | Aldehyde | 4.2E-08 |
| | | | | family | | | | | | Dehydrogenase | |
| SC0008 | SEQ ID NO:8 | smorf032 | 85 | Inositol 1,4,5-trisphosphate- | — | Atp_Gtp_A | — | 1 | 2.2E-39 | — | — |
| | | | | binding protein | | | | | | | |
| SC0009 | SEQ ID NO:9 | smorf044 | 77 | Tetracydine resistance protein | — | — | — | 1 | — | — | — |
| | | | | TetB signature | | | | | | | |
| SC0010 | SEQ ID NO:10 | smorf046 | 105 | Multicopper oxidase type 1 | — | Rnp_1 | — | 2 | 0.032 | — | — |
| SC0011 | SEQ ID NO:11 | smorf053 | 78 | Amphiphysin signature | — | — | — | 2 | — | — | — |
| SC0012 | SEQ ID NO:12 | smorf054 | 62 | Beta G-protein (transducin) | — | — | — | 1 | — | — | — |
| | | | | signature | | | | | | | |
| SC0013 | SEQ ID NO:13 | smorf057 | 111 | Paxillin signature | — | — | Coiled-coil | — | 0.012 | — | — |
| SC0014 | SEQ ID NO:14 | smorf066 | 219 | SUR2-type | — | — | — | — | 1.9E-111 | — | — |
| | | | | hydroxylase/desaturase | | | | | | | |
| | | | | catalytic domain | | | | | | | |
| SC0015 | SEQ ID NO:15 | smorf068 | 107 | Ribosomal protein L1 | — | — | — | 2 | 1.4E-46 | — | — |
| SC0016 | SEQ ID NO:16 | smorf070 | 132 | Formin signature | — | — | — | — | 3.1E-56 | — | — |
| SC0017 | SEQ ID NO:17 | smorf079 | 61 | C-C chemokine receptor type 9 | PLDc | — | — | — | 1.2E-13 | — | — |
| | | | | signature | | | | | | | |
| SC0018 | SEQ ID NO:18 | smorf080 | 213 | Telomere reverse transcriptase | — | | — | 4 | 2.5E-63 | — | — |
| | | | | signature | | Prokar_Lipoprotein | | | | | |
| SC0019 | SEQ ID NO:19 | smorf082 | 126 | eRF1-like proteins | RF1 | — | — | — | 2.2E-39 | Eukaryotic | 0.0013 |
| | | | | | | | | | | Peptide Chain | |
| | | | | | | | | | | Release Factor | |
| | | | | | | | | | | Subunit | |
| SC0020 | SEQ ID NO:20 | smorf093 | 78 | GTE/NF-I family | — | — | — | — | 0.0038 | — | — |
| SC0021 | SEQ ID NO:21 | smorf098 | 71 | Late protein L2 | — | — | — | 1 | — | — | — |
| SC0022 | SEQ ID NO:22 | smorf100 | 84 | Acyl-CoA oxidase | — | — | — | 1 | — | — | — |
| SC0023 | SEQ ID NO:23 | smorf101 | 56 | Xeroderma pigmentosum group | — | — | — | 1 | — | — | — |
| | | | | B protein signature | | | | | | | |
| SC0024 | SEQ ID NO:24 | smorf102 | 102 | Ribosomal protein L5 signature | Idh_C | — | Coiled-coil | — | 6.3E-42 | — | — |
| SC0025 | SEQ ID NO:25 | smorf103 | 92 | Arabidopsis thaliana 130.7kDa | — | — | Coiled-coil | — | 5.9E-30 | — | — |
| | | | | predicted protein structure | | | | | | | |
| SC0026 | SEQ ID NO:26 | smorf104 | 87 | Expansin/Lol pl family | — | — | — | — | — | — | — |
| | | | | signature | | | | | | | |
| SC0027 | SEQ ID NO:27 | smorf108 | 109 | Phosphoribosylglycinamide | — | — | — | 2 | 2.6E-45 | — | — |
| | | | | synthetase | | | | | | | |
| SC0028 | SEQ ID NO:28 | smorf109 | 78 | Carboxypeptidase Taq (M32) | — | — | — | — | 1E-11 | Interleukin-10- | 0.004 |
| | | | | metallopeptidase structure | | | | | | Chain_ | |
| SC0029 | SEQ ID NO:29 | smorf112 | 72 | Protein of unknown function | — | — | — | 1 | — | — | — |
| | | | | DUF133 | | | | | | | |
| SC0030 | SEQ ID NO:30 | smorf118 | 78 | MA3 domain | — | — | — | — | — | Methionyl-tRNA | 0.039 |
| | | | | | | | | | | Fmet | |
| | | | | | | | | | | Formyltransferase | |
| SC0031 | SEQ ID NO:31 | smorf121 | 86 | Barnase signature | — | — | — | — | 1.8E-11 | — | — |
| SC0032 | SEQ ID NO:32 | smorf122 | 93 | Saposin A-type domain | — | — | — | 2 | 0.027 | — | — |
| SC0033 | SEQ ID NO:33 | smorf123 | 58 | G-protein coupled receptors | — | — | — | — | — | — | — |
| | | | | family 3 (Metabotropic | | | | | | | |
| | | | | glutamate receptor-like) | | | | | | | |
| SC0034 | SEQ ID NO:34 | smorf127 | 69 | Aminoglycoside | — | — | — | 1 | — | — | — |
| | | | | phosphotransferase | | | | | | | |
| SC0035 | SEQ ID NO:35 | smorf137 | 99 | R3H domain | — | — | Coiled-coil | 1 | 7.6E-46 | — | — |
| SC0036 | SEQ ID NO:36 | smorf139 | 93 | Uncharacterized protein family | — | — | — | — | 1.1E-12 | — | — |
| | | | | UPF0030 | | | | | | | |
| SC0037 | SEQ ID NO:37 | smorf140 | 133 | Class IE cytochrome C | rrm | — | — | — | 2.4E-65 | — | — |
| | | | | signature | | | | | | | |
| SC0038 | SEQ ID NO:38 | smorf144 | 91 | Cytochrome c-type biogenesis | — | — | — | 1 | 0.0038 | — | — |
| | | | | protein CcbS signature | | | | | | | |
| SC0039 | SEQ ID NO:39 | smorfiSi | 84 | Uncharacterized protein family | UPF0057 | — | — | 2 | 1.4E-39 | — | — |
| | | | | UPF0057 | | | | | | | |
| SC0040 | SEQ ID NO:40 | smorf154 | 97 | Na+ /H + exchanger signature | — | — | — | 2 | — | — | — |
| SC0041 | SEQ ID NO:41 | smorf167 | 103 | Lysophosphatidic acid receptor | — | — | — | 1 | — | — | — |
| | | | | family signature | | | | | | | |
| SC0042 | SEQ ID NO:42 | smorf171 | 127 | Napin signature | — | — | — | 2 | — | — | — |
| SC0043 | SEQ ID NO:43 | smorf172 | 94 | Endogenous opiolds | — | — | — | — | 1.1E-42 | — | — |
| | | | | neuropeptides precursors | | | | | | | |
| SC0044 | SEQ ID NO:44 | smorf181 | 121 | RepA family | — | — | — | 1 | 2.9E-46 | — | — |
| SC0045 | SEQ ID NO:45 | smorf189 | 104 | Transforming growth factor | — | — | — | 3 | — | — | — |
| | | | | (TGF) beta family | | | | | | | |
| SC0046 | SEQ ID NO:46 | smorf201 | 82 | Prokaryotic DNA | — | — | — | — | 0.021 | Nadp(H)- | 0.036 |
| | | | | topoisomerase I | | | | | | Dependent | |
| | | | | | | | | | | Ketose | |
| | | | | | | | | | | Reductase | |
| SC0047 | SEQ ID NO:47 | smorf207 | 75 | Ribosomal L29e protein family | Ribosomal_L29e | — | — | — | 4.8E-28 | — | — |
| SC0048 | SEQ ID NO:48 | smorf217 | 102 | Uncharacterized protein family | UPF0021 | — | — | — | 1.6E-34 | — | — |
| | | | | UPF0021 | | | | | | | |
| SC0049 | SEQ ID NO:49 | smorf226 | 66 | Frizzled protein signature | — | Prokar_Lipoprotein | — | 1 | 0.034 | — | — |
| SC0050 | SEQ ID NO:50 | smorf247 | 74 | Zn-finger in ubiquitin- | — | — | — | — | — | — | — |
| | | | | hydrolases and other proteins | | | | | | | |
| SC0051 | SEQ ID NO:51 | smorf250 | 77 | Fibrillar collagen C-terminal | — | — | — | 1 | 0.0049 | Murine Minute | 0.025 |
| | | | | domain | | | | | | Virus Coat | |
| | | | | | | | | | | Protein | |
| SC0052 | SEQ ID NO:52 | smorf268 | 66 | Phosphoglucomutase and | — | — | — | — | 9E-27 | — | — |
| | | | | phosphomannomutase family | | | | | | | |
| SC0053 | SEQ ID NO:53 | smorf274 | 78 | Slow voltage-gated potassium | — | — | — | — | 0.036 | — | — |
| | | | | channel signature | | | | | | | |
| SC0054 | SEQ ID NO:54 | smorf279 | 129 | Glycoside hydrolase family 28 | — | — | — | — | 1.1E-44 | — | — |
| SC0055 | SEQ ID NO:55 | smorf283 | 81 | Iodothyronine deiodinase | — | — | — | 2 | 0.027 | — | — |
| SC0056 | SEQ ID NO:56 | smorf286 | 49 | Intron encoded nuclease repeat | — | — | — | — | — | — | — |
| SC0057 | SEQ ID NO:57 | smorf288 | 65 | 60Kd inner membrane protein | — | — | — | 1 | — | — | — |
| | | | | signature | | | | | | | |
| SC0058 | SEQ ID NO:58 | smorf294 | 116 | Membrane attack complex | — | — | — | — | 3.1E-40 | — | — |
| | | | | components/perforin/complement | | | | | | | |
| | | | | C9 | | | | | | | |
| SC0059 | SEQ ID NO:59 | smorf298 | 68 | Salmonella virulence plasmid | — | — | — | 1 | — | — | — |
| | | | | 28.1kDa A protein signature | | | | | | | |
| SC0060 | SEQ ID NO:60 | smorf301 | 105 | Maltose binding protein | — | — | — | — | — | — | — |
| | | | | signature | | | | | | | |
| SC0061 | SEQ ID NO:61 | smorf303 | 121 | DUF202 | — | — | — | 3 | 7.2E-18 | — | — |
| SEQID | SEQ ID NO: | smorf# | AA | desc | desc | desc | desc | tm domain | p-value | desc | p-value |
| SC0062 | SEQ ID NO:62 | smorf313 | 113 | K-Cl co-transporter signature | LHC | — | — | 2 | 0.000018 | — | — |
| SC0063 | SEQ ID NO:63 | smorf315 | 99 | PIN (PiIT N terminus) domain | — | — | — | — | 2.7E-41 | — | — |
| SC0064 | SEQ ID NO:64 | smorf318 | 59 | DAHP synthetase classll | — | — | — | 1 | — | — | — |
| SC0065 | SEQ ID NO:65 | smorf323 | 97 | Pi-dass glutathione S- | — | — | — | — | 3.3E-24 | — | — |
| | | | | transferase signature | | | | | | | |
| SC0066 | SEQ ID NO:66 | smorf324 | 73 | NADH-ubiqui- oxidoreductase | — | — | — | 1 | 0.024 | — | — |
| | | | | chain 5 signature | | | | | | | |
| SC0067 | SEQ ID NO:67 | smorf327 | 92 | Granins (chromogranin or | — | — | — | — | 7.8E-44 | — | — |
| | | | | secretogranin) | | | | | | | |
| SC0068 | SEQ ID NO:68 | smorf337 | 92 | Interleukin-1 receptor type II | — | — | — | — | 0.043 | — | — |
| | | | | precursor signature | | | | | | | |
| SC0069 | SEQ ID NO:69 | smorf350 | 104 | EDG-5 sphingosine 1- | — | Atp_Gtp_A | — | — | 4.2E-52 | — | — |
| | | | | phosphate receptor signature | | | | | | | |
| SC0070 | SEQ ID NO:70 | smorf352 | 65 | NADH-ubiqui- oxidoreductase | — | — | — | 1 | — | — | — |
| | | | | chain 5 signature | | | | | | | |
| SC0071 | SEQ ID NO:71 | smorf363 | 77 | Filoviridae VP35 signature | — | — | — | — | — | — | — |
| SC0072 | SEQ ID NO:72 | smorf382 | 74 | Lipoprotein amino terminal | — | — | — | — | — | — | — |
| | | | | region | | | | | | | |
| SC0073 | SEQ ID NO:73 | smorf392 | 131 | GNS1/SUR4 family | — | Prokar_Lipoprotein | — | 1 | — | — | — |
| SC0074 | SEQ ID NO:74 | smorf398 | 65 | Cytochrome B-245 heavy chain | — | — | — | — | — | — | — |
| | | | | signature | | | | | | | |
| SC0075 | SEQ ID NO:75 | smorf421 | 51 | Domain of unknown function | — | — | — | — | — | — | — |
| | | | | DUF34 | | | | | | | |
| SC0076 | SEQ ID NO:76 | smorf439 | 93 | Type II fibronectin collagen- | — | — | — | — | 7.2E-18 | — | — |
| | | | | binding domain | | | | | | | |
| SC0077 | SEQ ID No:77 | smorf483 | 94 | Uncharacterized protein family | — | — | — | — | 5.5E-13 | — | — |
| | | | | UPF0038 | | | | | | | |
| SC0078 | SEQ ID NO:78 | smorf494 | 53 | Bleomycin resistance protein | — | — | — | — | — | — | — |
| | | | | signature | | | | | | | |
| SC0079 | SEQ ID NO:79 | smorf499 | 81 | Vacuolating cytotoxin | — | — | — | 1 | — | — | — |
| SC0080 | SEQ ID NO:80 | smorf505 | 89 | Delta endotoxin | — | — | — | 1 | 0.033 | — | — |
| SC0081 | SEQ ID NO:81 | smorf508 | 251 | Ribonuclease III family | — | — | Coiled-coil | — | 8.3E-127 | — | — |
| SC0082 | SEQ ID NO:82 | smorf509 | 146 | FY-rich domain N-terminus | — | — | — | — | 4.9E-58 | — | — |
| SC0083 | SEQ ID NO:83 | smorf511 | 78 | YGGT family | — | — | — | 1 | — | — | — |
| SC0084 | SEQ ID NO:84 | smorf514 | 97 | Histone H5 signature | — | — | — | — | 0.016 | Dnaj | 0.025 |
| SC0085 | SEQ ID NO:85 | smorf519 | 107 | Ribosomal protein S27a | — | Prenylation | — | — | 0.0063 | — | — |
| SC0086 | SEQ ID NO:86 | smorf523 | 66 | Influenza virus nucleoprotein | — | — | — | 1 | 7.8E-28 | — | — |
| | | | | (NP) | | | | | | | |
| SC0087 | SEQ ID NO:87 | smorf526 | 68 | Zeta-tubulin signature | — | — | — | 1 | — | — | — |
| SC0088 | SEQ ID NO:88 | smorf530 | 110 | Protein of unknown function | — | — | — | 2 | 2.9E-46 | — | — |
| | | | | DUF55 | | | | | | | |
| SC0089 | SEQ ID NO:89 | smorf532 | 92 | Sodium | — | — | — | 2 | — | — | — |
| SC0090 | SEQ ID NO:90 | smorf540 | 73 | Coagulin signature | — | — | — | 2 | 0.021 | — | — |
| SC0091 | SEQ ID NO:91 | smorf543 | 91 | Cloacin immunity protein | G-patch | — | — | — | 3.4E-11 | — | — |
| | | | | signature | | | | | | | |
| SC0092 | SEQ ID NO:92 | smcrf544 | 79 | Cysteinyl leukotriene receptor | — | — | — | — | — | — | — |
| | | | | family signature | | | | | | | |
| SC0093 | SEQ ID NO:93 | smorf556 | 75 | Thaumatin family | — | — | — | — | — | — | — |
| | | | | (Pathogenesis-related protein) | | | | | | | |
| SC0094 | SEQ ID NO:94 | smorf561 | 77 | Apple domain | — | — | — | — | — | — | — |
| SC0095 | SEQ ID NO:95 | smorf564 | 163 | Mitochondnal energy transfer | mito_carr | — | — | 3 | 4.3E-75 | — | — |
| | | | | proteins (carrier protein) | | | | | | | |
| SC0096 | SEQ ID NO:96 | smorf570 | 113 | Calcium channel signature | — | Prokar_Lipoprotein | — | 2 | 2.7E-18 | — | — |
| SC0097 | SEQ ID NO:97 | smorf572 | 91 | Ubiquitin domain | ubiquitin | — | — | — | 1.2E-33 | Ubiquitin Core | 0.0004 |
| | | | | | | | | | | Mutant 1D7 | |
| SC0098 | SEQ ID NO:98 | smorf577 | 73 | Prokaryote metallothionein | — | — | — | 1 | — | — | — |
| | | | | signature | | | | | | | |
| SC0099 | SEQ ID NO:99 | smorf580 | 59 | Phosphoinositide 3-kinase C2 | — | — | — | — | 0.0054 | — | — |
| | | | | domain | | | | | | | |
| SC0100 | SEQ ID NO:100 | smorf587 | 75 | Histone H5 signature | — | — | — | 1 | 2.8E-32 | — | — |
| SC0101 | SEQ ID NO:101 | smorf590 | 86 | Orbivirus N53 | — | — | — | 1 | — | — | — |
| SC0102 | SEQ ID NO:102 | smorf591 | 111 | Coronavirus 51 glycoprotein | Adeno_Penton_{—} | Atp_Gtp_A | — | — | 0.0079 | — | — |
| | | | | | B | | | | | | |
| SC0103 | SEQ ID NO:103 | smorf598 | 94 | Glycoside hydrolase family 19 | DUF139 | — | — | — | — | — | — |
| SC0104 | SEQ ID NO:104 | smorf601 | 128 | Galactokinase | — | — | — | 2 | — | — | — |
| SC0105 | SEQ ID NO:105 | smorf605 | 72 | Protein of unknown function | — | — | — | 1 | — | — | — |
| | | | | DUF133 | | | | | | | |
| SC0106 | SEQ ID NO:106 | smorf621 | 177 | Protein of unknown function | HTH_3 | — | — | — | 4.5E-64 | — | — |
| | | | | DUF16 | | | | | | | |
| SC0107 | SEQ ID NO:107 | smorf625 | 80 | Levivirus coat protein | — | — | — | — | 0.027 | — | — |
| SC0108 | SEQ ID NO:108 | smorf626 | 120 | Nuclear transport factor 2 | — | — | — | 1 | — | — | — |
| | | | | (NTF2) | | | | | | | |
| SC0109 | SEQ ID NO:109 | smorf631 | 95 | Avidin / Streptavidin | — | — | — | 2 | — | — | — |
| SC0110 | SEQ ID NO:110 | smorf632 | 75 | Sulphonylurea receptor family | — | — | — | — | — | — | — |
| | | | | signature | | | | | | | |
| SC0111 | SEQ ID NO:111 | smorf640 | 116 | Kv1.6 voltage-gated K + | — | — | — | 1 | — | — | — |
| | | | | channel signature | | | | | | | |
| SC0112 | SEQ ID NO:112 | smorf643 | 85 | Alpha-2-macroglobulin family | — | — | — | 1 | — | — | — |
| SC0113 | SEQ ID NO:113 | smorf644 | 135 | Ribosomal protein L36 | Ribosomal_L36 | Ribosomal_L36 | — | — | 3.6E-46 | L36 Ribosomal | 9.7E-10 |
| | | | | | | | | | | Protein | |
| SC0114 | SEQ ID NO:114 | smorf655 | 66 | Glucokinase | — | — | — | — | — | — | — |
| SC0115 | SEQ ID NO:115 | smorf660 | 88 | Hemagglutinin esterase | — | — | — | 1 | 3.2E-31 | — | — |
| SC0116 | SEQ ID NO:116 | smorf664 | 150 | G-protein coupled receptors | — | — | — | 4 | 2E-52 | — | — |
| | | | | family 2 (secretin-like) | | | | | | | |
| SC0117 | SEQ ID NO:117 | smorf667 | 88 | S-crystallin signature | — | — | — | — | — | — | — |
| SC0118 | SEQ ID NO:118 | smorf669 | 54 | Fungal pheromone STE3 | — | — | — | 1 | 7.5E-23 | — | — |
| | | | | GPCR signature | | | | | | | |
| SC0119 | SEQ ID NO:119 | smorf672 | 85 | Bacterial thioester dehydrase | — | — | — | 1 | — | — | — |

What is claimed is:

1. A method of identifying open reading frames (ORFs) in a genome of an organism comprising the steps of:
(A) collecting a genomic sequence of a first organism;
(B) comparing the genomic sequence of the first organism to one or more other genomic libraries comprising genomes of other organisms containing ORFs; and
(C) determining ORFs for the first organism based on the comparison.

2. The method of claim 1, wherein the method uses a Basic Local Alignment Search Tool (BLAST) program.

3. The method of claim 2, wherein the p-value for the BLAST program is less than 1.

4. The method of claim 1, wherein the method uses a FASTA program or its equivalent.

5. The method of claim 1, wherein the step of collecting genomic sequences excludes sequences comprising known ORFs of the first organism.

6. The method of claim 1, wherein the first organism is a plant, a virus, a bacterium, a vertebrate, or an invertebrate.

7. The method of claim 6, wherein the first organism is a vertebrate selected from the group consisting of primate, equine, bovine, caprine, ovine, porcine, feline, canine, lupine, camelid, cervidae, rodent, avian and ichthyes.

8. The method of claim 7, wherein the primate is a human.

9. The method of claim 1, wherein the first organism is a fungi.

10. The method of claim 9, wherein the first organism is a fungi selected from the group consisting of oomycota, chytridiomycota, zygomycota, ascomycota, basidiomycota and deuteromycota.

11. The method of claim 10, wherein the ascomycota is Saccharomyces or Schizosaccharomyces.

12. The method of claim 11, wherein the Schizosaccharomyces is S. pombe.

13. The method of claim 11, wherein the Saccharomyces is Saccharomyces cerevisiae.

14. The method of claim 1, wherein the smORF encodes a polypeptide less than 100 amino acids long.

15. The method of claim 1, wherein the smORF encodes a polypeptide of 17 to 100 amino acids.

16. A method of identifying coding open reading frames (ORFs) of an organism comprising the steps of:
(A) collecting genomic sequences of a first organism;
(B) identifying stop-to-stop ORFs of the first organism;
(C) translating the stop-to-stop ORFs into polypeptide sequences;
(D) comparing the polypeptide sequences of the first organism to amino acid translations of genomic libraries comprising genomes of other organisms; and
(E) identifying, based on sequence identity, ORFs of the first organism that are present in the other organisms, wherein the identified ORFs are coding ORFs.

17. The method of claim 16, wherein the method uses a BLAST program.

18. The method of claim 17, wherein the BLAST program uses a p-value less than 1.

19. The method of claim 16, wherein the method uses a FASTA program.

20. The method of claim 16, wherein method excludes previously identified ORFs of the first organism.

21. The method of claim 16, wherein the first organism is an eukaryote or a prokaryote.

22. The method of claim 21, wherein the first organism is the eukaryote is a vertebrate selected from the group consisting of primate, equine, bovine, caprine, ovine, porcine, feline, canine, lupine, camelid, cervidae, rodent, avian, and ichthyes.

23. The method of claim 22, wherein the primate is a human.

24. The method of claim 16, wherein the first organism is a fungi.

25. The method of claim 24, wherein the first organism is a fungi selected from the group consisting of oomycota, chytridiomycota, zygomycota, ascomycota, basidiomycota and deuteromycotoa.

26. The method of claim 25, wherein the ascomycota is Saccharomyces or Schizosaccharomyces.

27. The method of claim 26, wherein the Schizosaccharomyces is S. pombe.

28. The method of claim 26, wherein the Saccharomyces is Saccharomyces cerevisiae.

29. A smORF selected from SEQ ID NOS:1-119.

30. A smORF selected from the group of sequences consisting of smORF18 (SEQ ID NO: 4), smORF570 (SEQ ID NO: 96), smORF139 (SEQ ID NO: 36), smORF57 (SEQ ID NO: 13) or a biologically active fragment thereof, and optionally, a sequence required for an amplification reaction.

31. A smORF identified using the method of claim 1.

32. A vector comprising the smORF of claim 31.

33. A cell comprising the vector of claim 32.

34. A smORF encoding a polypeptide selected from the group consisting of SEQ ID NOS: 674-1345.

35. A smORF encoding a polypeptide of smORF18 (SEQ ID NO: 677), smORF57 (SEQ ID No: 776), smORF139 (SEQ ID NO: 799), or smORF570 (SEQ ID NO: 814).

36. An isolated polypeptide encoded by the smORF of claim 31.

37. A nucleic acid that hybridizes to a sense or an antisense strand of the smORF of claim 31.

38. An isolated polypeptide comprising SEQ ID NOS: 674-1345 or 1346.

39. The isolated polypeptide of claim 36, wherein the polypeptide comprises SEQ ID NOS: 674-791 or 792.

40. An isolated polypeptide selected from the group consisting of smORF18 (SEQ ID NO: 677) and smORF 57 (SEQ ID NO: 776).

41. An antisense compound comprising 15 to 50 nucleobases, wherein at least 8 contiguous nucleobases are derived from a nucleic acid sequence selected from SEQ ID NO: 1-119.

42. The antisense compound of claim 41, wherein the at least 8 contiguous nucleobases are selected from smORF18 (SEQ ID NO: 4) and smORF57 (SEQ ID NO: 13).

43. The antisense compound of claim 41, wherein the antisense compound is an antisense oligonucleotide.

44. The antisense compound of claim 41, wherein the oligonucleotide comprises at least one modified internucleoside linkage.

45. The antisense compound of claim 41, wherein the oligonucleotide is a chimeric oligonucleotide.

46. The antisense compound of claim 43, wherein the antisense oligonucleotide comprises at least one modified nucleobase.

47. The antisense compound of claim 43, wherein the antisense oligonucleotide comprises a modified internucleoside linkage, a phosphorothioate linkage, a modified sugar moiety, or a modified nucleobase.

48. A method of inhibiting the expression of a smORF encoding a protein from Table 2 comprising administering an antisense compound which binds to a corresponding nucleic acid of Table 2.

49. A method of identifying an inhibitory compound to a protein encoded by the ORF identified by claim 1 comprising the steps of:
(a) contacting the protein encoded by the ORF or a biologically active fragment of the protein with a compound under conditions effective to promote specific binding between the protein and the compound; and
(b) determining whether the protein or biologically active fragment thereof bound to the compound; and
(c) determining whether the compound that binds to the protein further inhibits the activity of the protein.

50. The method of claim 47, wherein the compound is a library selected from a group consisting of a combinatorial small organic library, a phage display library and a combinatorial peptide library.

51. A polypeptide or biologically active fragment thereof comprising at least 10 contiguous amino acids of SEQ ID NOS: 674-1346.

52. A composition comprising the polypeptide or biologically active fragment thereof of claim 51 and a pharmaceutically acceptable carrier.

53. An antibody or immunologically active fragment thereof which recognizes and binds to a polypeptide or fragment of the polypeptide of claim 51.

54. The antibody of claim 53, wherein the antibody is a human antibody, a humanized antibody, a primatized antibody, a monoclonal antibody or a bispecific antibody.

55. The immunologically active fragment of the antibody of claim 53, wherein the fragment is Fab, Fab′, F(ab′)₂, Fv, scFv, and Fd.

56. The antibody of claim 53, wherein the antibody recognizes and binds to a polypeptide selected from the group consisting of SEQ ID NOS: 674-792.

57. The antibody of claim 53, wherein the antibody binds to the protein of smORF18, smORF57, smOR139, smORF570.

58. A pharmaceutical composition comprising a nucleic acid of claim 29 and a pharmaceutically acceptable excipient.

59. A pharmaceutical composition comprising a polypeptide of claim 38 and a pharmaceutically acceptable excipient.
